(19) United States
(12) Patent Application Publication
(10) Pub. No.: US 2022/0073931 A1
(43) Pub. Date: Mar. 10, 2022

(54) ANTISENSE OLIGONUCLEOIDES OF GLUTATHIONE S-TRANSFERASES FOR CANCER TREATMENT

(71) Applicants: ATSO CORPORATE AFFAIRS, S.A. DE C.V., Ciudad de Mexico (MX); CAS BIOTECHNOLOGY, S.C., Cuernavaca, Morelos (MX)

(72) Inventor: Alberto Checa Rojas, Cuernavaca (MX)

(21) Appl. No.: 17/310,354

(22) PCT Filed: Jan. 30, 2020
(86) PCT No.: PCT/MX2020/000003

§ 371 Date: Jul. 28, 2021

(30) Foreign Application Priority Data
Jan. 31, 2019 (MX) MX/A/2019/001389

(51) Int. Cl. C12N 15/113; G01N 33/574

(52) U.S. Cl. CPC C12N 15/1137; G01N 33/57492; G01N 2333/91177; C12N 2320/30; C12N 2310/11

(57) ABSTRACT

The present invention relates to the identification of glutathione S transferase in tumors containing the same to be treated to inhibit protein expression of GSTs proteins and induce cell death and decrease tumor volume.

### FIELD OF THE INVENTION

The present invention is based on the identification of glutathione S transferase (GSTs) in carcinogenic tumors and a novel treatment for mammals, which inhibits the protein expression of GSTs proteins. The treatment is carried out through the use of antisense oligonucleotides directed to the messenger ribonucleic acids (mRNA) of the GSTM3 and GSTP1, leading to a reduced proliferation of cancer cells and a decrease in tumor progression. In addition, this reduction in proliferation extends to cancers resistant to conventional therapies.

### BACKGROUND OF THE INVENTION

According to the World Health Organization (WHO), cancer is a generic term that designates a broad group of diseases that can affect any part of the body; they are also called malignant tumors or malignancies. A feature that defines cancer is an altered cell division extending beyond its usual limits, being able to invade adjacent parts of the body or spread to other organs, a process called metastasis. Metastases are the leading cause of death from cancer.

The WHO considers that the most common cancers are lung, breast, colorectal, prostate, stomach, liver, esophagus, cervical, thyroid, bladder, non-Hodgkin lymphoma, pancreas, leukemia, kidney, uterine body, oropharynx, cerebral and central nervous system, ovarian, melanoma, gallbladder, larynx, multiple myeloma, nasopharyngeal, laryngopharynx, Hodgkin lymphoma, testicles, salivary glands, vulva, Kaposi sarcoma, penis, mesothelioma, and vaginal.

Therefore, there is a need for drugs and treatments for mammals that have even been diagnosed with cancer.

As an example, one of the most common treatments for cervical cancer includes a chemoradiation therapy based on cisplatin. In addition, this treatment is regularly the only option to treat cervical cancer in advanced stages, and in most cases the disease cannot be eradicated (see Green, J. A., Kirwan, J. M., Tierney, J. F., Symonds, P., Fresco, L., Collingwood, M., & Williams, C. J. (2001). Survival and recurrence after concomitant chemotherapy and radiotherapy for cancer of the uterine cervix: a systematic review and meta-analysis. Lancet, 358(9284), 781-786.

There is a vast literature and patent application documents related to the treatment of cancers, for example, U.S. patent application Ser. No. 15/270,774 of ZHI-MING ZHENG et al., Refers to polynucleotide markers that can be detected two and can be used for the diagnosis of pre-cancers associated with the Human Papillomavirus and cancers associated with the Human Papillomavirus, such as cervical cancer and cervical intraepithelial malignancies as well as methods of treating such cancers. However, said document, like others, does not indicate the treatment of cancers in mammals already diagnosed with them.

Mexican patent application No. MX/a/2014/004285 refers to a method for the diagnosis of cervical cancer comprising performing an electrophoretic polyacrylamide gel run of a serum sample of a subject suspected of having cervical cancer, as well as determining the presence of at least one protein of molecular weight selected from the group of 60 kDa and 50 kDa. This document is intended to diagnose cervical cancer early, but not to treat mammals that have already been diagnosed with cervical cancer.

European Patent No. EP1531843 refers to the hematological study in oncoginecology, which can be used in patients with cervical cancer recurrence to evaluate the effectiveness of anti-tumor treatment and predict the course of the tumor process. The method makes it possible to carry out the selection of the most important rational method of antitumor impact and minimize the development of a number of side effects. Said document does not describe selecting candidates for said treatment to improve its effectiveness.

Patent document No. JP2005189228 provides a method and kit for diagnosing cancers including non-small cell lung cancer, esophageal cancer, laryngeal cancer, pharyngeal cancer, lingual cancer, stomach cancer, kidney cancer, large intestine cancer, cervical cancer, brain tumor, pancreatic cancer and bladder cancer, which are provided with an immunological technique that uses monoclonal antibodies against AKR1B10. Said document claims a protein consisting of the amino acid sequence of SEQ ID NO:1 in a biological sample, or a protein consisting of an amino acid sequence in which one or more amino acids are deleted, substituted or added in the amino acid sequence of SEQ ID NO:1 and has an activity of aldocete lactase. The protein is one that has the amino acid sequence shown in SEQ ID NO:1, which is detected by an immunological method. The document refers to diagnosing the types of cancer mentioned there, but not treating a mammal that has already been diagnosed with cancer.

GSTs are a family of enzymes that exhibit various functions, including the detoxification of xenobiotic compounds, evasion of the immune system and inhibition of apoptosis. In various types of cancer, it has been reported that members of the glutathione S-transferase (GST) family are overexpressed and in most cases they are related to a poor prognosis and resistance to chemotherapy (see Cabelguenne et al., 2001; Huang, Tan, Thiyagarajan, & Bay, 2003; Meding et al., 2012; Pectasides, Kamposioras, Papaxoinis, & Pectasides, 2008). In particular, it has been reported that GSTP1 and GSTM3 are deregulated in cancer cells, such as: triple negative breast cancer, prostate cancer, lung cancer and colorectal cancer (see Loktionov, a, Watson, M. a, Gunter, M., Stebbings, W. S., Speakman, C. T., & Bingham, S. a. (2001) Glutathione-S-transferase gene polymorphisms in colorectal cancer patients: interaction between GSTM1 and GSTM3 allele variants as a risk-modulating factor Carcinogenesis, 22(7), 1053-1060.

In addition, it is known that the GSTP1 protein plays a regulatory role through interaction with the TRAF2 protein and decreased signal transduction of the receptors in the TNF-α and JNK pathways, which are responsible for the activation of the apoptosis (see Adler, V., Yin, Z., Fuchs, S. Y., Benezra, M., Rosario, L., Tew, K. D., Ronai, Z. (1999). Regulation of JNK signaling by GSTp. The EMBO journal, 18(5), 1321-1334). On the other hand, it has been observed that the overexpression of GSTM3 in colon cancer is considered a marker of regional lymph node metastases (see Meding, S., Balluff, B., Elsner, M., Schöne, C., Rauser, S., Nitsche, U., . . . Walch, A. (2012). Tissue-based proteomics reveals FXYD3, S100A11 and GSTM3 as novel markers for regional lymph node metastasis in colon cancer. Journal of Pathology, 228(4), 459-470), and the subexpression of GSTM3 in urinary bladder cancer is associated with longer survival (see Mitra, A. P., Pagliarulo, V., Yang, D., Waldman, F. M., Datar, R. H., Skinner, D. G., . . . Cote, R. J. (2009). Generation of a concise gene panel for outcome prediction in urinary bladder cancer. Journal of Clinical Oncology, 27(24), 3929-3937).

Recently antisense molecules capable of inhibiting gene expression with great specificity have been used and, due to this, many research efforts related to the modulation of gene expression by antisense oligonucleotides (OAS) are being made. Some of these OAS focus on the inhibition of specific genes such as oncogenes or viral genes. Antisense oligonucleotides are directed against RNA (sense chain) or against DNA, wherein they form triple structures that inhibit transcription by RNA polymerase II. To achieve a desired effect on the negative regulation of the specific gene, oligonucleotides should promote the decomposition of the targeted mRNA or block the translation of that mRNA, thus avoiding de novo synthesis of the unwanted target protein (see US 20120029060 A1)

International publication No. WO2017091885, which describes the use of antisense oligonucleotides, provides compounds, compositions and methods for modulating the expression of the monocarboxylate transporter 4 (MCT4). In particular, this invention relates to antisense oligonucleotides (OAS) capable of modulating the expression of human MCT4 mRNA and uses and methods thereof for the treatment of various indications, including various cancers. In particular, the invention relates to therapies and treatment methods for cancers such as prostate cancer, including castration-resistant prostate cancer (CRPC).

Protein overexpression of GSTM3 and GSTP1 proteins during tumor progression (hereinafter identified as PT) play a regulatory role through interaction with proteins, for example TRAF2/6 proteins and, therefore, a evasion of signal transduction of apoptosis activation, favoring cell survival and PT (Wu Y, Fan Y, Xue B, Luo L, Shen J, Zhang S, Jiang Y, Yin Z. Human glutathione S-transferase P1-1 interacts with TRAF2 and regulates TRAF2-ASK1 signals. Oncogene. 2006; 25: 5787-800. Doi: 10.1038/sj.onc.1209576, Although GSTM3 interacts with TRAF6). In addition, GST's expression is involved in the modulation of detoxification processes in cancer cells and, therefore, participates in the chemoresistance response of conventional therapies in the cancer patients.

### SUMMARY OF THE INVENTION

The present invention relates to the identification of GSTM3 and/or GSTP1 proteins in cancers, to provide a treatment with antisense oligonucleotides directed to said proteins (GSMT3 and GSTP1) to mammals identified as candidates for said treatment. At least one or more of said combined oligonucleotides block a protein specifically or both proteins.

According to a first aspect, the invention is directed to GSTM3 and GSTP1 proteins to be used as therapeutic targets and/or prognostic factors for mammals with cancer.

In the present invention, xenografted cancer cell lines in immunosuppressed mice were used to analyze, through their proteomics, the differences in protein expression during tumor progression. In this analysis it was found that glutathione S transferase P1 and M3 (hereafter referred to as GSTP1 and GSTM3, respectively) were some of the proteins that consistently increased their levels during tumor growth. In addition, it was found that through “inhibition of genes” (knock-down) of said proteins, they play a critical role for cell survival and tumor progression. Also, the abundance of the levels of these proteins in cancer biopsies was correlated with the survival of the patients. Therefore, it was shown that GSTP1 and GSTM3 proteins are also useful for prognostic purposes and that they are excellent candidates for target gene therapy for cancer.

In a second aspect, the present invention provides the use of antisense oligonucleotides of glutathione S transferases, preferably GSTM3 and GSTP1, without being limited thereto, for the treatment of cancers, in candidate subjects who have previously been diagnosed with cancer, wherein antisense oligonucleotides are between 15-50 nucleotides in length.

In another aspect, the present invention provides a method of treatment for cancer comprising: a) the extraction of the protein from the tumor tissue, b) carrying out an analysis by immunodetection techniques such as, for example, spotting of bands by western (Western blot), immunohistochemistry, ELISA, etc., to identify if the tumor has GSTM3 and/or GSTP1 proteins and c) administer the antisense oligonucleotides for said proteins.

In another aspect, the present invention provides a kit for identifying a candidate subject to be treated with the oligonucleotides of the present invention comprising at least one antisense oligonucleotide of the glutathione S transferases, preferably GSTM3 and GSTP1, without being limited thereto., a protein extraction solution, at least two antibodies for the identification of GSTM3 and GSTP1 proteins and optionally a secondary antibody, and a colorimetric developing solution for western (western blot) or immunohistochemistry (IHQ) staining.

Additional aspects and advantages of the present invention will be better understood by persons skilled in the art in light of the detailed description and with reference to the following figures.

### BRIEF DESCRIPTION OF THE FIGURES

FIG. 1A is a schematic representation of the experimental design to analyze the proteome dynamics of tumors in a murine model of HeLa and SiHa cell lines.

FIG. 1B shows the kinetics of tumor growth of the HeLa (yellow) and SiHa (blue) cell lines. The kinetic endpoints (30, 45 and 50 days) were used to perform the proteomic analysis.

FIG. 1C shows that GSTM3 was identified in HeLa tumors and GSTP1 in SiHa tumors in 2-D electrophoresis. Expression levels of both proteins were confirmed by immunoblot analysis.

FIGS. 1D-1F show a representative image of each protein on days 30, 45 and 50. (1D) 14 proteins with constant expression in HeLa and SiHa tumors; (1E) 3 proteins with a subexpression over time in HeLa and SiHa; (1F) 17 proteins with different expression between HeLa and SiHa tumors.

FIGS. 2A-2C show an analysis made through the GeneCodis website of the enrichment of the gene ontology of the proteins identified in the tumor of HeLa and SiHa cells. (2A) Biological processes enriched in overregulated shared proteins; (2B) biological processes enriched in constant shared proteins; (2C) biological processes enriched in shared downregulated proteins.

FIGS. 3A-3F show that GSTM3 interacts with TRAF6 in cervical cancer tumors HeLa and SiHa, under physiological conditions. (3A) Cytoscape interaction network representing the prey interactions of GSTM3; (3B) co-immunoprecipitation analysis of the GSTM3 and TRAF6; (3C) Western spotting for TRFA6, ERK, pERK, NF-κ, PNF-κB; IKBα, p38, pp38, JNK, pJNK and TLR4 in the protein extracts of HeLa and SiHa tumors; (3D) Proportional Venn diagram of proteins secreted in cervical cancer cell lines with 264 common proteins; (3E) identification of two proteins secreted in vitro that can activate the TLR4 signal pathway; HSP60 and HSP70; (3F) Western spotting of TRL4 activators; HSP70 and HSP60 in cervical cancer tumors, HSP60 secreted in SiHa and HeLa tumors on day 50, and HSP70 protein secreted in SiHa tumors at 45 days and HeLa tumors at 30 and 50 days.

FIGS. 3G-3H show workflows to obtain secreted proteins in vivo or ex vivo.

FIGS. 4A-4F show that GSTM3 interacts with HPV18 E7 wherein GST and E7 provide survival advantages to cells exposed to stress conditions. (4A) The overlap of GSTP1 and GSTM3 proteins show high structural similarities (green-orange), unconserved structures (gray) and the structure of the HPV18 E7 protein (blue) using the HPV16 E7 as a mold; (4B) interaction of the human recombinant ScGSTM3 N-6× his-tag protein with the E7 of the HPV18 protein; (4C) HeLa cells were transfected with a plasmid expressing HE718C-6× his-tag; (4D) shows PAEP assays; (4E) survival test with 6.0 mM cisplatin; (4F) PAEP assays using the MDA cell line with 6.0 mM cisplatin.

FIGS. 4G-4H show that MDA-MB-231 is a negative cell line for HPV18 and GSTs proteins (GSTM3 and GSTP1) (4G), (4H) show the expression of GSTM3 and GSTP1 proteins in breast tumor cuts (of MDA) and colon cancer (COLO 237) generated in mice of the Nu/Nu strain.

FIGS. 4I-4L show the construction of yeast plasmids, transformation and expression of recombinant protein. (4I) Human recombinant protein of GSTM3 with a histidine (His) tag that was expressed in the yeast Saccharomyces cerevisiae; (4J) after capturing the recombinant GSTM3, it was incubated with a HeLa cell protein extract (positive for HPV18); (4K) HPV18 E7 recombinant protein with a His expression in the HeLa cell line; (4L) after capturing the E7 of the 6× his-tag recombinant HPV18 with nickel beads, it is incubated.

FIGS. 5A-5E show that “gene inhibition” (knock-down) of GSTM3 and GSTP1 affects the viability of cervical cancer cell lines in culture. (5A) Genetic experiments of “gene inhibition” (knock-down) of GSTM3 and GSTP1 in HeLa cells (yellow lines) and HaCaT (blue lines); (5B) viability assay with OAS-control or OAS-GST (at 640 ng/mL) determined by staining with violet crystal; (5C) live/dead cell assays determined by staining SYTO 9 (live cells, green color and dead, red cells) in cells treated with OAS-control and OAS-GST (640 ng/mL); (5D-E) GST inhibition with antisense oligonucleotide treatment in cervical cancer cell lines.

FIGS. 6A-6E show how “gene inhibition” (knock-down) of GSTM3 and GSTP1 affects tumor progression (TP) in cervical cancer tumors.

FIG. 6F shows the growth kinetics of cell lines with and without SFB indicating that there are no significant differences when the cells reach 70% confluence on the sixth day.

FIGS. 7A-7B illustrate that HeLa tumors treated with OAS-GSTM3 show inactivation of ERK and p65 NF-κB proteins.

FIGS. 7C-7D show that in CaLo tumors only pERK was inactivated after treatment with any of the antisense oligonucleotides for GST.

FIGS. 7E-7F show that for SiHa tumors only NF-κB was inactivated by any of the treatments.

FIGS. 7G-7H show that in CaSki tumors both proteins were inactivated after any treatment.

FIGS. 8A-8D shows the correlation between GST protein expression and survival of patients with cervical cancer. (8A) Representative specimens of invasive cervical cancer with different levels of expression of GSTM3 and GSTP1 (weak, moderate and high); (8B) ROI of GSTM3 and GSTP1 in 13 patients with cervical cancer evaluated; (8C) the percentage of ROI of GSTP1 was classified as weak (10%), moderate (20-50%) and high (51-100%); (8D) Kaplan-Meier survival graph, for the advanced stage of cervical cancer according to the levels of protein expression of GSTM3 and GSTP1 (log-rank test, p<0.05).

FIGS. 8E-8F shows the expression of GSTM3 and GSTP1 in cervical cancer in the terminal stage.

FIG. 9 shows that during the progression of cervical cancer several processes such as cell survival, proliferation and evasion of apoptosis through the MAPK kinase and NF-κB pathways are stimulated by the presence of GSTM3 and/or GSTP1. The “gene inhibition” (knock-down) of GSTM3 and GSTP1 affects the activation of activated apoptosis through the activation of JNK and p38 or the phosphorylated inhibition of NF-κB and ERK.

### DETAILED DESCRIPTION OF THE INVENTION

As indicated above, the word cancer is a generic term that designates a broad group of diseases that can affect any part of the body, such as lung cancer, breast cancer, colorectal cancer, prostate cancer, stomach cancer, liver cancer, esophageal cancer, cervical cancer, thyroid cancer, bladder cancer, non-Hodgkin lymphoma, pancreatic cancer, leukemia, kidney cancer, uterine body cancer, oropharyngeal cancer, brain and central nervous system cancer, ovarian cancer, melanoma cancer, gallbladder cancer, larynx cancer, multiple myeloma cancer, nasopharyngeal cancer, laryngopharyngeal cancer, Hodgkin lymphoma, testicular cancer, salivary gland cancer, vulvar cancer, Kaposi sarcoma, penile cancer, mesothelioma, and vaginal cancer, among others, so it should be understood that a person skilled in the art will appreciate that the invention described below is susceptible to variations and modifications different from those specifically described and therefore, the present invention includes all such variations and modifications as well as all the stages, features, compositions and compounds referred to or indicated therein, whether in individual or collective and any of all combinations or any of two or more of the stages or features.

Furthermore, it should also be understood that the present invention is not limited in scope to the specific embodiments described therein, which are proposed for exemplification purposes only. Functionally equivalent products, compositions, combinations and methods as well as their application in mammals are clearly within the scope of the invention, as described.

Tumor progression (hereinafter also referred to as PT) involves changes in the deregulation of metabolic and cellular processes. The study of tumor proteome dynamics represents the protein changes of this deregulation mechanism during PT. Therefore, the study of proteome dynamics for example in cervical cancer (hereinafter also referred to as CC) will provide relevant information to understand PT and the disease to be treated.

Protein overexpression of the glutathione S transferase GSTM3 and GSTP1 genotypes during tumor progression (PT) plays a regulatory role through interaction with proteins, for example TRAF2/6 and therefore an evasion of transduction of signals of apoptosis activation, favoring cell survival and PT. In addition, GST expression is involved in the modulation of detoxification processes in cancer cells and, therefore, participates in the survival response to conventional chemotherapy in patients with CC and other types of cancer.

The present invention relates to antisense oligonucleotides of glutathione S transferase (GSTs) such as GSTM3 and GSTP1, as novel candidates for use as therapeutic targets and/or prognostic factors for patients (mammals) who have been diagnosed with CC and other types of cancer.

The identification of candidate subjects for the treatment of cancers and their treatment is carried out by identifying the protein expression of glutathione S transferase proteins (GSTs). Treatment includes the use of antisense oligonucleotides targeting the messenger ribonucleic acids (mRNA) of the GSTM3 and GSTP1, which give tumor cells greater resistance to chemotherapies.

The antisense oligonucleotide (OAS) is preferably any antisense oligonucleotide, which reduces the expression levels of the GSTMs and thus increases the sensitivity of the cell, tissue and/or organ to the chemotherapeutic agent in vitro, ex vivo, or in vivo.

In a first embodiment, the antisense oligonucleotide is an oligonucleotide composed of subunits called “nucleotides”, wherein each nucleotide is made up of three parts: a sugar or a functional analogue thereof, a nitrogen base and a functional group that serves as an internucleotide bond (usually a phosphate group) between the subunits that make up the oligonucleotide. Each of these components may contain the following modifications:

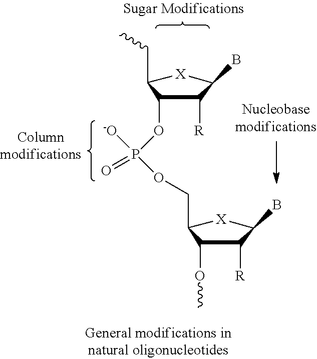

wherein

X means a sugar, ribose in the case of RNA or deoxyribose in the case of DNA or the functional analog thereof in the nucleotide.

B means the nitrogenous base bound to sugar or a functional analogue thereof, and R means the functional group in carbon 2′ of sugar when it is ribose.

In a second embodiment, modifications to ribose sugar, without being limiting, are:

Modifications in the 2′ position of ribose sugar that include the replacement of the OH group by different groups among which are (2′-O-methyl (2′OMe), 2′-O-methoxyethyl (2′-OMOE), 2′fluor (2′-F) and 2′-chiral fluor (2′-F))

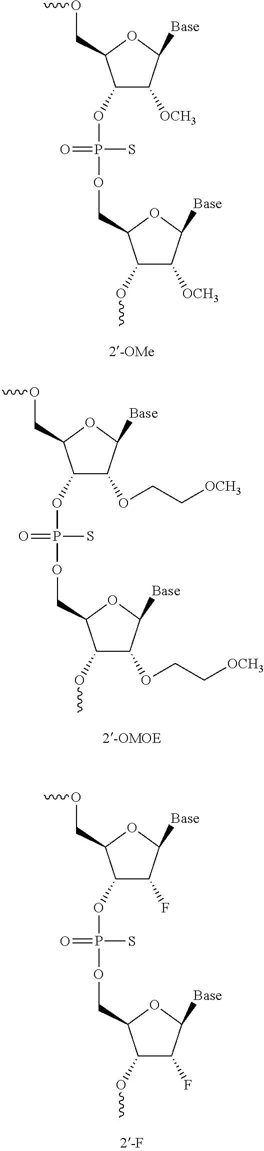

wherein base refers to the nitrogenous base of the nucleotide.

In a third embodiment, modifications in ribose include the use of an analogue of bicyclic ribose, wherein the ribose structure contains an additional ring, for example, ribose with 2′-O, 4′-C-methylene (LNA-blocked nucleic acid), 2′-O, 4′-C-oxymethylene, 2′-O, 4′-C-methylene-β-D-ribofuranosil rings, among others.

Additionally, the substitution of ribose sugar can be carried out by analogous functional groups with similar function, for example, the substitution of the ribose by another sugar such as arabinose, morpholino, or by ribose analogs with a spirocyclic ring in different positions of the sugar ring, without being such limiting examples.

The following are examples of some of the different modifications to the sugary part of the oligonucleotide.

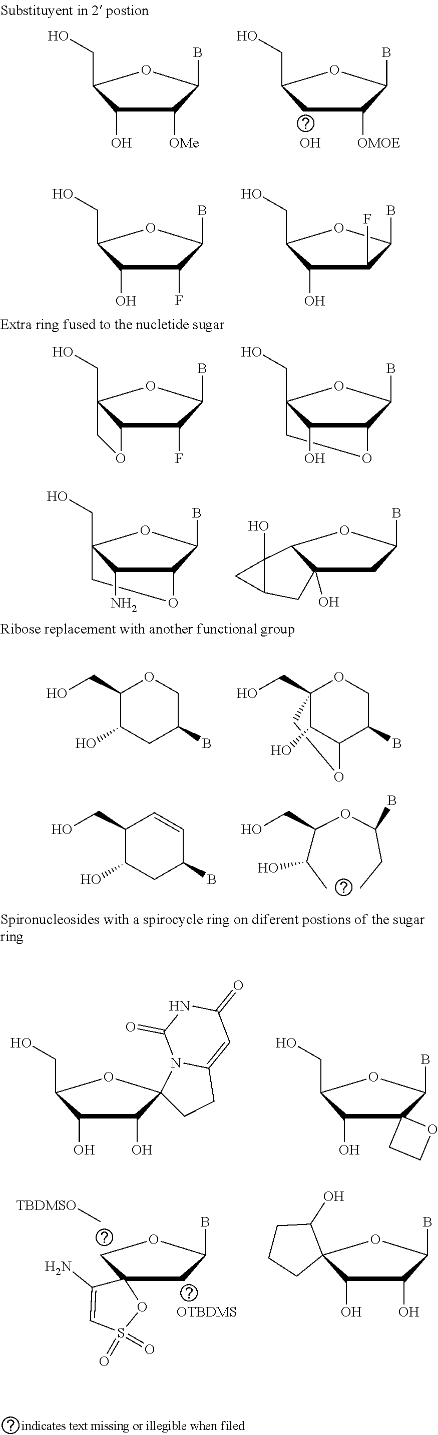

In a fourth embodiment, modifications to the nucleobases or nitrogenous bases include, but are not limited to, adenine, cytosine, guanine, thymine, as well as their modifications 5-methylcytosine, 2-aminopurine, 2-amino-6-chloropurine, 2-diaminopurine, hypoxatin, 5-propynyl uracil, 2-thio thymine, N3-thioethyl thymine, 3-deaza adenine, 8-azido adenine and 7-deaza guanine, or the use of universal bases such as 3-nitropyrrole, imidazole-4-carboxamide, 5-nitroindole.

The following are examples of some structures of different types of modified nucleobases and nucleobase analogs.

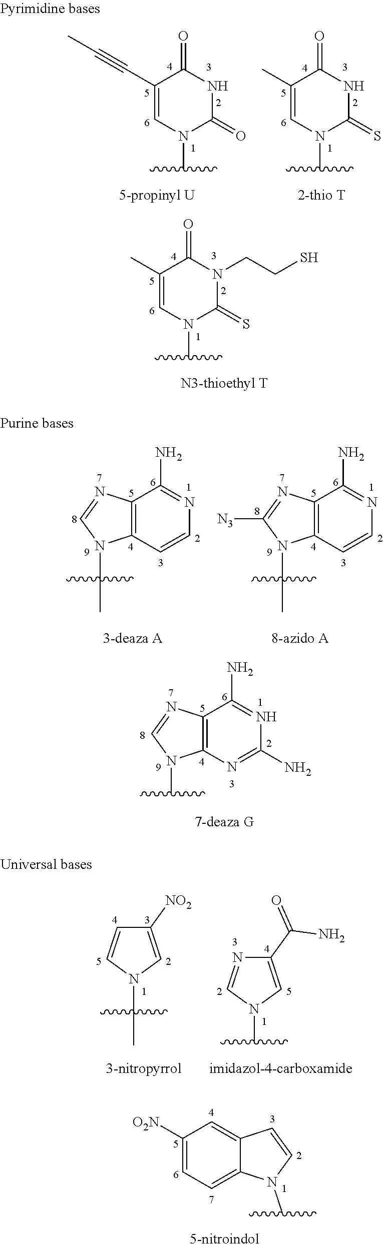

In a fifth embodiment, the modifications in the column or skeleton of the internucleoside groups, which bind them, can be but are not limited to:

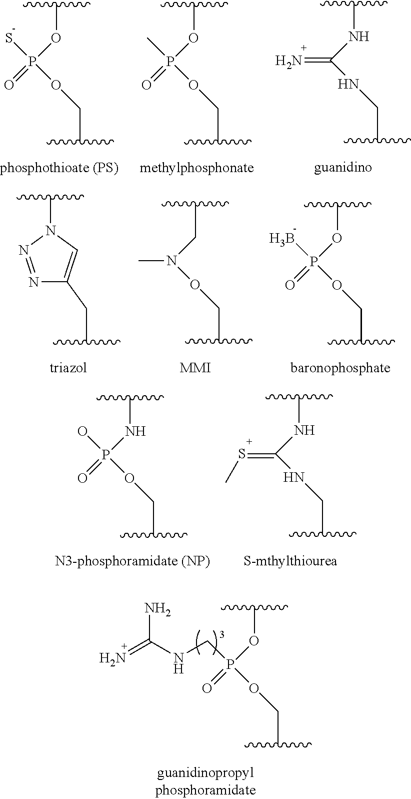

5′-N-carbamate, methylene-methylimine, amide, phosphorodithioate, thioether, thioformacetal and mercaptoacetamide. The substitution of the phosphodiester or phosphorothioate group can be carried out by phosphorodiamidate groups with piperazine and morpholino residues (PMOs).

For a person skilled in the art of the present invention it will be obvious that, within the scope, the chimeras resulting from the mixture of 2 or more chemical modifications mentioned above are also included, for example without being limiting, the replacement of the ribose by a morpholino ring and replacement of the phosphodiester group with phosphorodiamidate groups without charge (PMOs), the replacement of ribose and the phosphodiester bond is replaced by pseudopeptide N-(2 aminoethyl) glycine and the nucleobase is linked to the oligonucleotide column through of an ethyl enecarbonyl bond (PNA), a chimera where the ribose contains an alkyl substituent at the 2′ position and the phosphodiester bond is replaced by a phosphotiester bond, a chimera wherein the ribose is replaced by a ribose analogue with 2′-O,4′-C-methylene (LNA) rings and the phosphodiester bond is substituted by a phosphotiester bond, among others.

Examples of chimeras resulting from the combination of the different components of a nucleotide are by way of example:

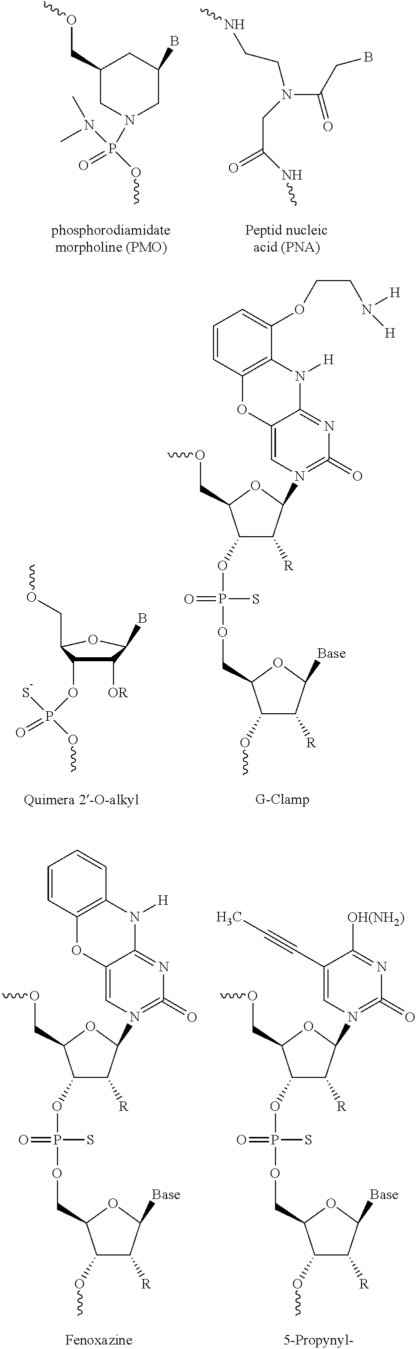

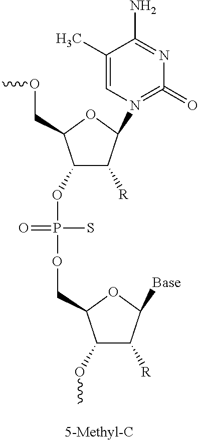

### DESCRIPTION OF THE PREFERRED EMBODIMENT

### Tumor Progression Model

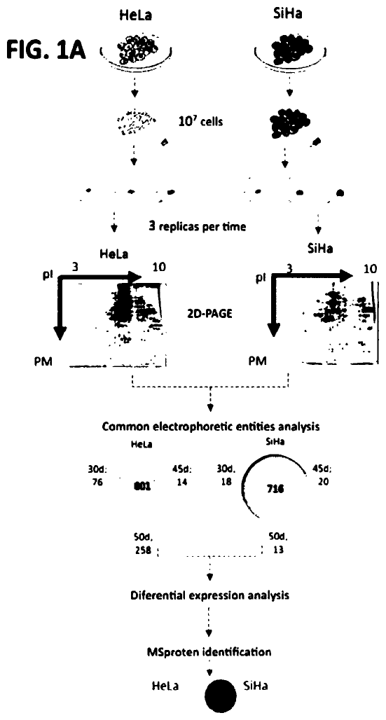

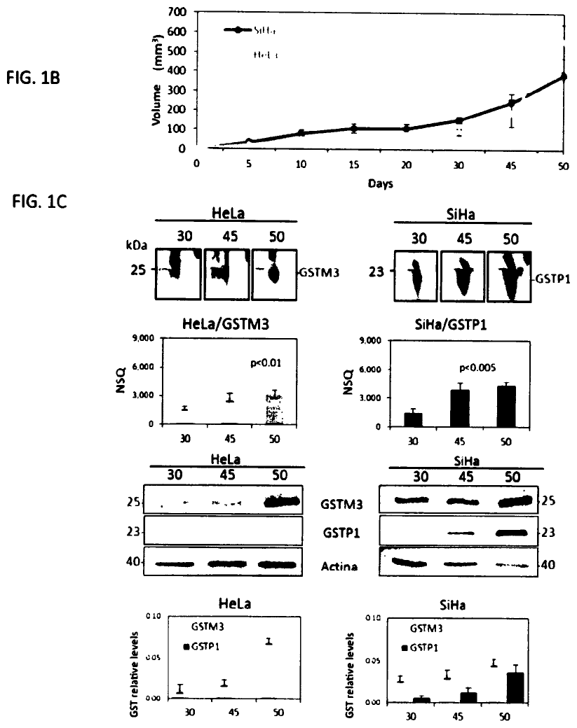

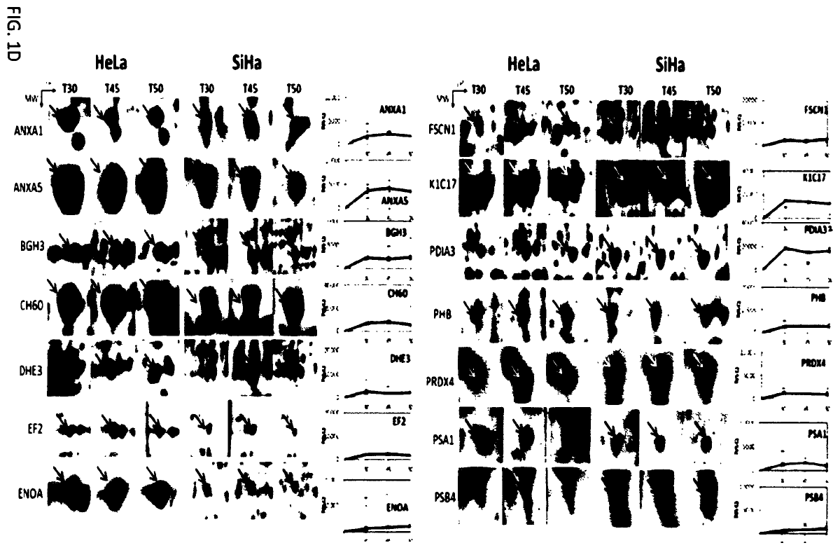

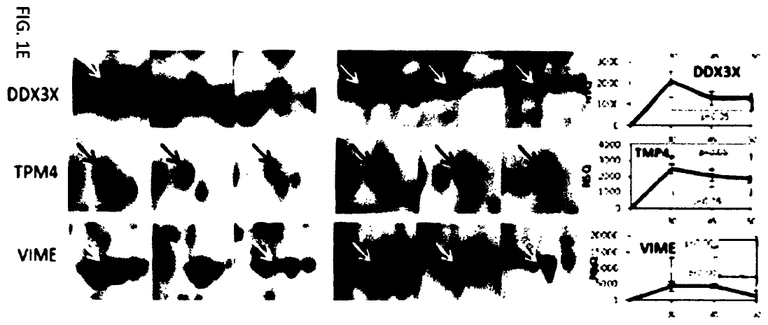

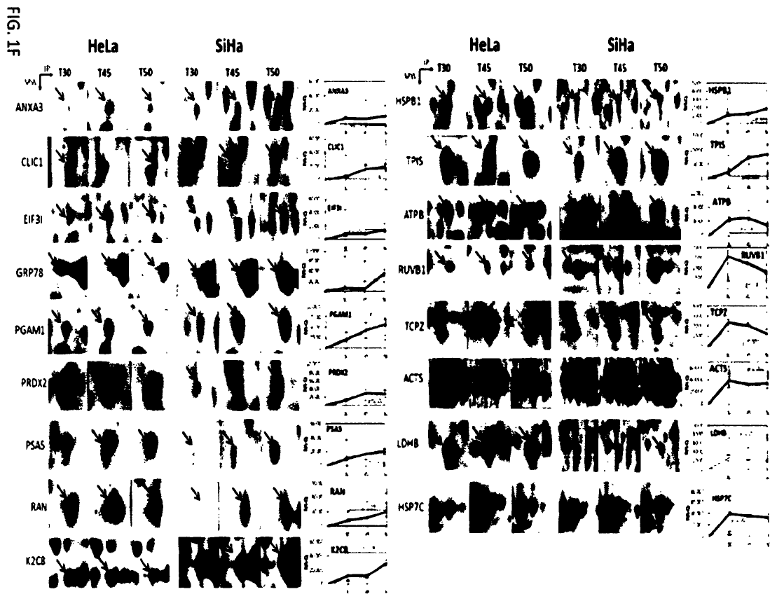

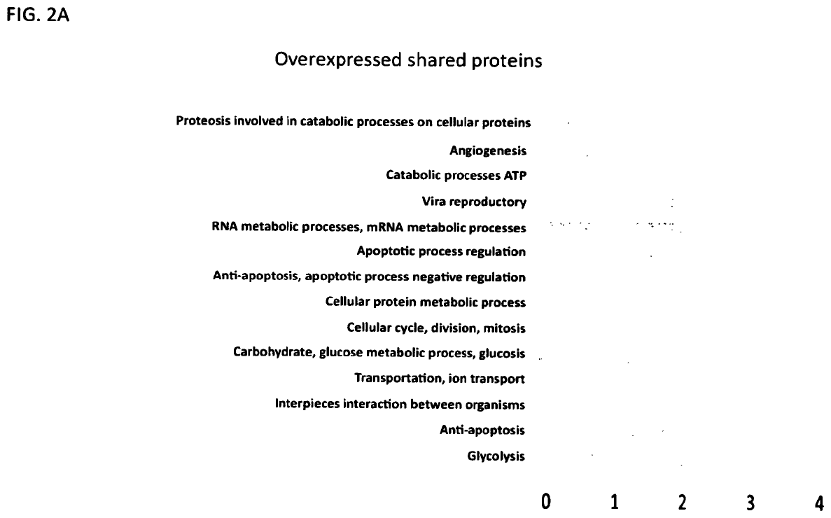

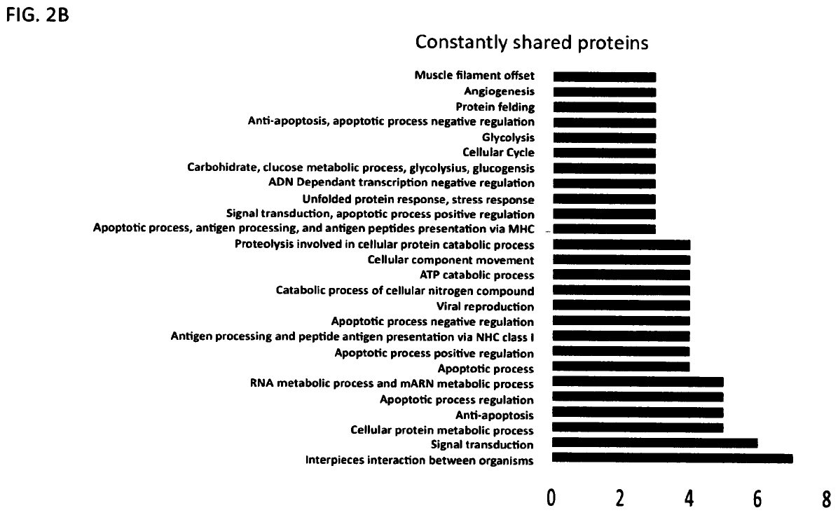

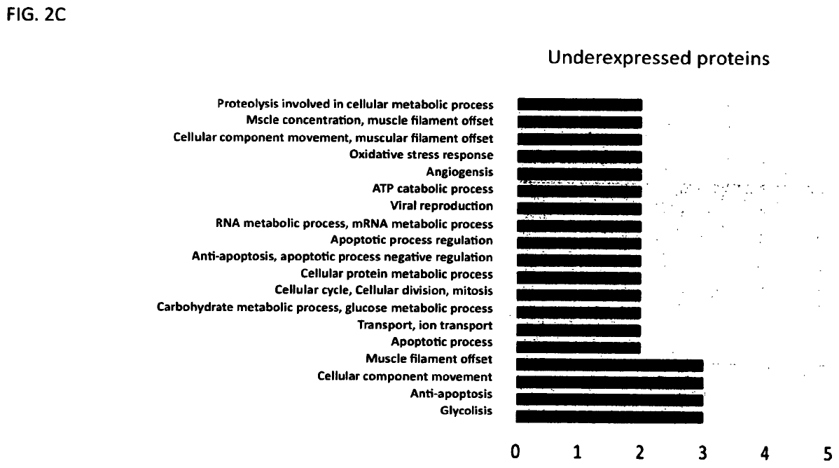

In order to create a suitable model to study tumor progression (PT), cervical cancer cell lines (SiHa and HeLa), triple negative breast (MDA-231-MB) and colon (COLO 205) were used to generate grafted tumors in mice. Cancer cells were cultured at 70% confluence and 10⁷ cells were inoculated into female mice of the Nu/Nu strain for 4 to 6 weeks. The tumor volume was measured according to the equation of the volume of an ellipsoid described as: Vtm=η/6 (L*W*H), where L: length, W: width and H: height, and were taken in seven different moments of PT, for example, a kinetics of HeLa and SiHa tumors was taken at 5, 10, 15, 20, 30, 45 and 50 days after inoculation. The first four measurement times showed a low progression of tumor growth. However, at the endpoints of kinetics, tumor volume grew exponentially for HeLa cell tumors. From day 30 to 45 the average tumor volume doubled and by day 50 the average volume was 3 times more than the previous measurement. For SiHa cells, tumor growth rates were lower than HeLa; from 30 to 45 days, SiHa tumors grew 1.6 times more, while, in the last five days, tumors grew on average 1.6 times larger (see FIGS. 1A-B). Due to these results, the dynamics of PT were evaluated more at the level of proteome between times 30, 45 and 50 days after inoculation.

Analysis of 2D-PAGE Gels and Protein Identification.

Tumors of the two types of cancer cells (HeLa and SiHa) were collected and total proteins were extracted for analysis by means of two-dimensional electrophoresis gels (2D-PAGE). The analysis of the 2D-PAGE images of each repetition for each time studied and for each type of cell was carried out using the PDQuest software. An average of 866 electrophoretic entities (spots or “stains”) were detected for samples of HeLa tumors at each repetition. For SiHa tumors, the average of spots or “stains” detected in 2D-PAGE images was 766. The correlation coefficient between repetitions for each tumor time and cell type was determined from the 2D-PAGE maps.

Table 1 below shows that the correlation coefficient in all tumors was greater than 0.7 in both types of cells. The proteomic profile was obtained each time and then compared to find differentiated proteins during PT.

TABLE 1

| | HeLa | SiHa |
|---|---|---|
| Time (days) | Stains/CCf* | Stains/CCf* |
| 30 | 824/0.710 | 765/0.731 |
| 45 | 763/0.723 | 768/0.836 |
| 50 | 1012/0.724 | 766/0.756 |

*CCf = correlation coefficient.

In addition, 601 points were detected in HeLa tumors in the three times of PT, while in SiHa tumors the number of common points was 716 (see FIG. 1A). For protein identification, a total of 90 tumor gel points was selected including both HeLa and SiHa cell types, based on their abundance patterns between the ages of the tumors. All electrophoretic or 2D gel entities were processed as described in the experimental section and identified after MALDI-TOF mass spectrometry analysis.

Table 2 below shows that 46 different proteins were identified from HeLa tumors, including 34 with constant expression through PT, 7 proteins showed a negative regulation along PT, 3 proteins increased their abundance during tumor growth, and 2 were found with an oscillating pattern.

TABLE 2
Total proteins expressed in HeLa tumors (days 30, 45 and 50)

| HeLa | ID Swissprot * | MASCOT Score** |
|---|---|---|
| Constant | ENOA_HUMAN | 92 |
| | ANXA1_HUMAN | 134 |
| | ANXA3_HUMAN | 148 |
| | ANXA5_HUMAN | 65 |
| | ATPB_HUMAN | 126 |
| | BGH3_HUMAN | 135 |
| | CH60_HUMAN | 74 |
| | CLIC1_HUMAN | 80 |
| | CP21A_HUMAN | 67 |
| | DHE3_HUMAN | 150 |
| | EF2_HUMAN | 178 |
| | EIF3I_HUMAN | 81 |
| | FSCN1_HUMAN | 126 |
| | GDIB_HUMAN | 83 |
| | GRDN_HUMAN | 73 |
| | GRP78_HUMAN | 189 |
| | GSTO1_HUMAN | 89 |
| | HSP71A_HUMAN | 78 |
| | HSPB1_HUMAN | 136 |
| | K1C17_HUMAN | 102 |
| | K2C8_HUMAN | 98 |
| | PDIA3_HUMAN | 165 |
| | PGAM1_HUMAN | 73 |
| | PHB_HUMAN | 136 |
| | PKHA2_HUMAN | 66 |
| | PRDX2_HUMAN | 75 |
| | PRDX4_HUMAN | 107 |
| | PSA1_HUMAN | 73 |
| | PSA5_HUMAN | 81 |
| | PSB4_HUMAN | 85 |
| | RAN_HUMAN | 73 |
| | RUVB1_HUMAN | 85 |
| | TCPZ_HUMAN | 109 |
| | TPIS_HUMAN | 102 |
| Downregulated | ACTS_HUMAN | 172 |
| | ANXA2_HUMAN | 68 |
| | DDX3X_HUMAN | 104 |
| | IF4A1_HUMAN | 134 |
| | TPM3_HUMAN | 65 |
| | TPM4_HUMAN | 106 |
| | VIME_HUMAN | 171 |
| Overexpressed | G3P_HUMAN | 79 |
| | GSTM3_HUMAN | 81 |
| | LDHB_HUMAN | 106 |
| Oscillation | HSP7C_HUMAN | 134 |
| | TPM2_HUMAN | 71 |

* Swiss-Prot database.
**MASCOT Score database

Table 3 below shows that a total of 44 proteins were identified from the SiHa cells. The identified proteins were distributed according to their expression pattern, 20 were found without differences in the three tumor ages evaluated, 8 decreased their abundance during PT, while 16 showed an increasing pattern. When analyzing all the proteins identified in tumors of both types of cells (Hela and SiHa), it was found that 34 proteins were shared between the two types of tumors, including 14 that show the same pattern of expression.

TABLE 3
Total proteins expressed in SiHa tumors (days 30, 45 and 50)

| SiHa | ID Swissprot | MASCOT Score |
|---|---|---|
| Constant | ACTS_HUMAN | 122 |
| | ACTG_HUMAN | 107 |
| | ANXA1_HUMAN | 132 |
| | ANXA5_HUMAN | 102 |
| | BGH3_HUMAN | 83 |
| | CH60_HUMAN | 122 |
| | DHE3_HUMAN | 80 |
| | EF2_HUMAN | 184 |
| | FSCN1_HUMAN | 108 |
| | HNRH3_HUMAN | 153 |
| | HSP71_HUMAN | 183 |
| | HSP7C_HUMAN | 134 |
| | K1C17_HUMAN | 192 |
| | LDHB_HUMAN | 111 |
| | PCNA_HUMAN | 114 |
| | PDIA3_HUMAN | 135 |
| | PHB_HUMAN | 131 |
| | PRDX4_HUMAN | 80 |
| | PSB4_HUMAN | 75 |
| | PSA1_HUMAN | 90 |
| downregulated | ATPB_HUMAN | 174 |
| | DDX3X_HUMAN | 134 |
| | GRP75_HUMAN | 75 |
| | K1C17_HUMAN | 176 |
| | RUVB1_HUMAN | 137 |
| | TPM4_HUMAN | 106 |
| | TCPZ_HUMAN | 158 |
| | VIME_HUMAN | 147 |
| Overexpressed | ACTB_HUMAN | 101 |
| | ANXA3_HUMAN | 168 |
| | CLIC1_HUMAN | 83 |
| | EIF3I_HUMAN | 99 |
| | ENOA_HUMAN | 81 |
| | ENOA_HUMAN* | 114 |
| | GSTP1_HUMAN | 74 |
| | GRP78_HUMAN | 154 |
| | HSPB1_HUMAN | 108 |
| | K1C17_HUMAN | 154 |
| | K2C8_HUMAN | 172 |
| | PGAM1_HUMAN | 135 |
| | PRDX2_HUMAN | 121 |
| | PSA5_HUMAN | 96 |
| | RAN_HUMAN | 113 |
| | TPIS_HUMAN | 81 |

*Swiss-Prot database.
**MASCOT Score database

Among the proteins with levels of overexpression, two family members were identified Glutathione S-transferase (GSTM3 and GSTP1). GSTM3 was identified in HeLa tumors and GSTP1 in SiHa tumors (see FIG. 1C). Expression levels of both proteins were confirmed by immunoblot analysis (see FIG. 1C). In addition, immunoblotting revealed that both proteins with over-expression patterns are observed in SiHa tumors. However, in HeLa tumors only overexpression was confirmed for GSTM3, and it was found that GSTP1 is not detectable at any stage of the tumor.

Bioinformatic Analysis

Subsequently, the proteins identified in both tumors were used to perform a functional enrichment analysis based on the biological processes of gene ontologies (Gene Ontology: GO.

The proteins were grouped according to their expression levels and subjected to an enrichment analysis. The results indicated that proteins that increase their levels during PT are mainly involved in anti-apoptotic, cell division, glycolysis, angiogenesis, viral reproduction and regulation of apoptotic processes (see Checa-Rojas A., et al. GSTM3 and GSTP1: novel players driving tumor progression in cervical cancer, Oncotarget. 2018; 9: 21696-21714). In addition, including all identified proteins, the results suggest that during PT the overrepresented pathways are related to the cellular response to stress, the MAPK6/MAPK4 and NIK/NF-kappaB signaling pathways.

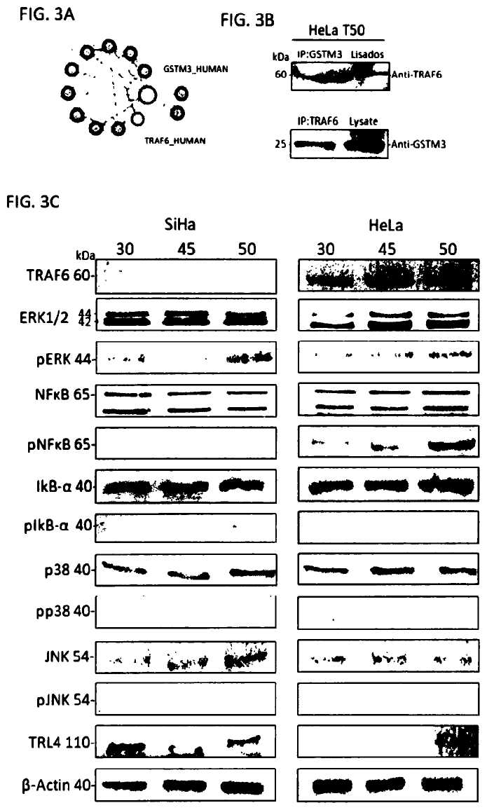

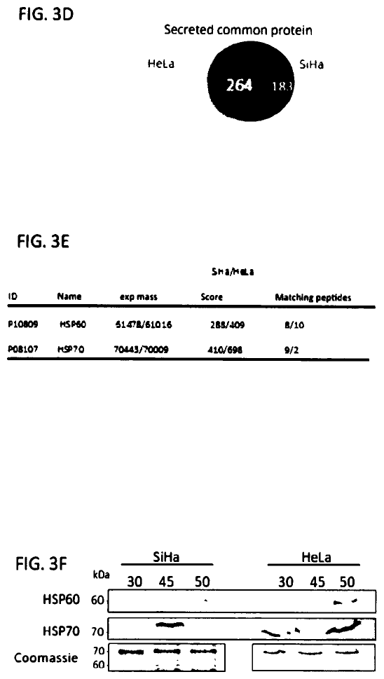

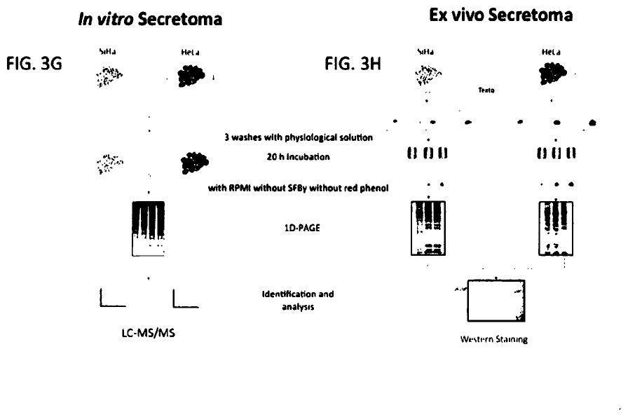

On the other hand, data mining analysis revealed that GSTM3 and GSTP1 interact with the proteins of factors associated with the tumor necrosis factor receptor (TRAF). However, said analysis can be extended to other proteins. Specifically, the interaction of GSTP1 with TRAF2 previously validated in HeLa cells (Wu, Y., Fan, Y., Xue, B., Luo, L., Shen, J., Zhang, S., . . . Yin, 2. (2006). Human glutathione S-transferase P1-1 interacts with TRAF2 and regulates TRAF2—ASK1 signals. Oncogene, 25, 5787-5800), and GSTM3 was reported as an interactor of TRAF6 (tumor necrosis factor receptor associated with factor 6) (Rouillard, A D, Gundersen, G W, Fernandez, N F, Wang, Z., Monteiro, C D, McDermott, M G, & Ma'ayan, A. (2016). The harmonizome: a collection of processed datasets gathered to serve and mine knowledge about genes and proteins. Database, 2016, baw100.) (see FIG. 3A).

To demonstrate that this interaction occurs under physiological conditions, TRAF6 expression was observed in both CC tumors (see FIG. 3B). It was observed that TRAF6 was expressed only in HeLa tumors. After that, the interaction analysis was carried out, by coimmunoprecipitation (IP). It was found that, GSTM3 co-immunoprecipitates with TRAF6 and vice versa (see FIG. 3B). It is shown that GSTM3 is associated with TRAF6 CC tumors.

Modulation of MAPK Signaling During PT

Given the importance of TRAF proteins on the downstream activation of the mitogen-activated protein kinase (MAPK) cascade, the phosphorylated version of NF-κB p65 (ser529), ERK, JNK and p38 by Western spotted (blot) of the PT proteins. The results showed that the phosphorylation of p38 and JNK was reduced over time in both CC tumors, but not in pNF-κB and pERK (see FIG. 3C). The results indicate that during PT the apoptotic processes are repressed and, therefore, cell proliferation is constantly activated via ERK and NF-κB. FIG. 3A shows the interaction network representing GSTM3 dam interactions, which can be visualized by the network limits. This analysis was performed to obtain the interactions reported by the SysBiomics database, in which it was observed that TRAF6 interacts with GSTM3. FIG. 3B shows the co-immunoprecipitation of GSTM3 and TRAF6. In the proportional Venn diagram of FIG. 3D the secreted proteins of the CC cell lines with 264 common proteins are were observed and FIG. 3E shows that two proteins identified in secreted proteins that can activate the TLR4 signal pathway expressed in vitro in HSP60 and HPS70. The western spotting of HSP70 and HSP60 activators of TRL4 in proteins secreted by CC tumors is shown in FIG. 3F. In addition, it is shown that HSP60 was expressed in SiHa and HeLa tumors at 50 days and HSP70 protein expressed in SiHa tumors at 43 days, and Hela tumors at 30 and 50 days.

Endogenous Secreted TLR4 Receptor Activators in the CC

On the other hand, it is known that the activation of the TLR4 pathway is driven by the presence of lipopolysaccharides (LPS) from bacterial infections, but also by endogenous activators such as HSP60 and HSP70 thermal shock proteins. To demonstrate that CC cell lines can express endogenous activators of the Toll type receptor 4 (TLR4), an in vitro analysis of the secreted proteins was performed using the HeLa and SiHa cell lines (see FIG. 3G). The following Tables 45 show the results obtained.

Secreted proteins were analyzed by LC-MS/MS and a total of 432 HeLa and 447 SiHa proteins were identified, of which 264 were common between both cell lines (see FIG. 3D). Among the endogenous activators reported for TLR4, two secreted proteins were identified as members of the family of thermal shock proteins, HSP60 and HSP70 for both cell lines (see FIG. 3E). To determine whether these proteins were also expressed during PT, the proteins ex vivo secreted in CC tumors were then analyzed by western spotting (see FIG. 3F, FIG. 3H). The results were correlated in in vivo and ex vivo experiments, indicating that secretion of HSP60 and HSP70 activates TLR4 signaling. See FIG. 3C.

Proteomic mass spectrometry data has been deposited in ProteomeXchange Consortium via the deposit of its associated PRoteomics IDEntifications (PRIDE) with the data set identifier PXD005466.

TABLE 4
Proteins secreted in HeLa cells

| | | | | Matching |
|---|---|---|---|---|
| ID | Name | Mass | Score | peptides |
| P62258 | 14-3-3 epsilon protein | 29155 | 321 | 8 |
| P61981 | 14-3-3 gamma protein | 28285 | 149 | 3 |
| P63104 | 14-3-3 zeta/delta protein | 27728 | 337 | 3 |
| Q9NQ66 | 1-phosphatidylinositol-4,5- | 0 | 51 | 4 |
| | bisphosphate | | | |
| | phosphodiesterase beta-1 | | | |
| Q4KWH8 | 1-phosphatidylinositol-4,5- | 0 | 40 | 4 |
| | bisphosphate | | | |
| | phosphodiesterase eta-1 | | | |
| O75038 | 1-phosphatidylinositol-4,5- | 0 | 37 | 4 |
| | bisphosphate | | | |
| | phosphodiesterase eta-2 | | | |
| P62081 | 40S ribosomal protein S7 | 22113 | 125 | 3 |
| P08865 | 40S Ribosomal Protein SA | 32833 | 38 | 2 |
| P32754 | 4-hydroxyphenylpyruvate | 44906 | 181 | 6 |
| | dioxygenase | | | |
| P10809 | 60 KDa thermal shock protein, | 61016 | 409 | 10 |
| | mitochondrial | | | |
| P05388 | 60S P0 ribosomal acidic | 34252 | 118 | 3 |
| | protein | | | |
| P62899 | 60S ribosomal protein L31 | 14454 | 54 | 2 |
| Q02878 | 60S ribosomal protein L6 | 32708 | 59 | 2 |
| P52209 | 6-phosphogluconate | 53106 | 245 | 2 |
| | dehydrogenase, | | | |
| | decarboxylation | | | |
| P11021 | Glucose-regulated 78 kDa | 72288 | 751 | 1 |
| | protein | | | |
| Q8IZT6 | Microcephaly-associated | 409540 | 52 | 6 |
| | protein similar to an | | | |
| | abnormal spindle | | | |
| Q9BWD1 | Acetyl-CoA | 41324 | 77 | 1 |
| | acetyltransferase, cytosolic | | | |
| P68032 | Actin, alpha 1 heart muscle | 41992 | 480 | 2 |
| P68133 | Actin, alpha skeletal muscle | 42024 | 480 | 2 |
| P62736 | Actin, aortic smooth muscle | 41982 | 93 | 2 |
| P60709 | Actin, cytoplasmic 1 | 41710 | 640 | 1 |
| P63261 | Actin, cytoplasmic 2 | 41766 | 640 | 1 |
| P25054 | Adenomatous polyposis coli | 311455 | 47 | 6 |
| | protein | | | |
| O00468 | Agrina | 214706 | 43 | 7 |
| Q2M3C7 | SPHKAP A-kinase anchor | 186339 | 33 | 4 |
| | protein | | | |
| P12814 | alpha-actinin-1 | 102993 | 579 | 13 |
| P35609 | alpha-actinin-2 | 103788 | 34 | 2 |
| O43707 | alpha-actinin-4 | 104788 | 1436 | 6 |
| P06733 | alpha-enolase | 47139 | 1172 | 6 |
| Q96L96 | alpha-protein kinase 3 | 201148 | 39 | 5 |
| Q8TCU4 | Alstrom 1 syndrome protein | 0 | 47 | 7 |
| Q01484 | Anquirin-2 | 429990 | 35 | 5 |
| Q12955 | Anquirin-3 | 480113 | 36 | 6 |
| P04083 | Annex A1 | 38690 | 49 | 1 |
| P07355 | Annex A2 | 38580 | 471 | 10 |
| P08758 | Annex A5 | 35914 | 251 | 5 |
| P46013 | KI-67 antigen | 358474 | 57 | 5 |
| O00203 | AP-3 beta-1 subunit complex | 0 | 34 | 3 |
| P04114 | Apolipoprotein B-100 | 515283 | 64 | 5 |
| O43150 | Arf-GAP with SH3 domain, ANK | 0 | 36 | 2 |
| | repeat and protein 2 | | | |
| | containing the PH domain | | | |
| P00966 | Argininosuccinate synthase | 46501 | 277 | 9 |
| P17174 | Aspartate aminotransferase, | 46219 | 388 | 8 |
| | cytoplasmic | | | |
| Q7Z591 | Transcription factor | 155044 | 36 | 3 |
| | containing AT connection | | | |
| Q5T9A4 | 3B protein containing AAA | 0 | 46 | 4 |
| | domain of the ATPase family | | | |
| Q96QE3 | Protein 5 containing the AAA | 0 | 38 | 4 |
| | domain of the ATPase family | | | |
| Q9BZC7 | Member 2 of subfamily A of | 269701 | 36 | 6 |
| | the cassette linking ATP | | | |
| Q8IZY2 | Member 7 of subfamily A of | 234201 | 37 | 3 |
| | the cassette linking ATP | | | |
| P53396 | ATP-citrate synthase | 120762 | 585 | 7 |
| 98160 | Heparan basement membrane | 468532 | 43 | 4 |
| | proteoglycan sulfate core | | | |
| | protein | | | |
| Q562R1 | Protein 2 similar to beta- | 0 | 256 | 1 |
| | actin | | | |
| P25098 | 1 beta-adrenergic kinase | 0 | 34 | 3 |
| | receptor | | | |
| P13929 | Beta-enolase | 46902 | 216 | 2 |
| Q96T60 | polynucleotide | 0 | 32 | 4 |
| | phosphatase/bifunctional | | | |
| | kinase | | | |
| Q8NFC6 | Biorientation of chromosomes | 0 | 42 | 6 |
| | in protein 1 of cell-similar | | | |
| | division | | | |
| O14514 | Brain specific angiogenesis | 0 | 40 | 4 |
| | inhibitor 1 | | | |
| O60241 | Brain specific angiogenesis | 0 | 33 | 3 |
| | inhibitor 2 | | | |
| O60242 | Guanine nucleotide exchange | 201909 | 32 | 4 |
| | protein 2 inhibited Brefeldin | | | |
| | A | | | |
| O60243 | Brevican core protein | 99056 | 43 | 2 |
| O60244 | Protein 1 containing WD and | 0 | 64 | 4 |
| | Bromodomain repetition | | | |
| O60245 | Carbamoyl phosphate synthase | 164835 | 133 | 3 |
| | [ammonia], mitochondrial | | | |
| O60246 | Cathepsin D | 44524 | 220 | 6 |
| O60247 | Cathepsin Z | 33846 | 167 | 4 |
| O60248 | ABL1 and CDK5 enzyme substrate | 0 | 41 | 1 |
| O60249 | Protein 2 associated with CDK5 | 0 | 35 | 3 |
| | regulation subunit | | | |
| O60250 | Centlein | 161504 | 47 | 3 |
| O60251 | F centromer protein | 367537 | 54 | 6 |
| O60252 | Protein E associated with | 0 | 54 | 5 |
| | centromer | | | |
| O60253 | Protein 350 associated with | 350716 | 45 | 5 |
| | centrosome | | | |
| O60254 | Chlorine intracellular channel | 26906 | 128 | 2 |
| | protein 1 | | | |
| O60255 | O-Acetyltransferase coline | 82483 | 33 | 4 |
| O60256 | CLIP association protein 1 | 0 | 66 | 4 |
| O60257 | Clusterin | 52461 | 205 | 3 |
| O60258 | Cofilin-1 | 18491 | 195 | 3 |
| O60259 | Protein 17 containing double | 0 | 37 | 2 |
| | helix domain | | | |
| O60260 | Protein 78 containing double | 0 | 42 | 3 |
| | helix domain | | | |
| O60261 | Protein 87 that contains | 0 | 44 | 4 |
| | double helix domain | | | |
| O60262 | Protein 93 containing double | 0 | 39 | 4 |
| | helix domain | | | |
| O60263 | Alpha-1 (III) collagen chain | 138479 | 33 | 4 |
| O60264 | Alpha-1 (VII) collagen chain | 295041 | 57 | 7 |
| O60265 | Alpha-1 (XII) collagen chain | 332941 | 1361 | 31 |
| O60266 | Alpha-1 (XIV) collagen chain | 0 | 47 | 1 |
| O60267 | Collagen alpha-1 (XXVII) chain | 0 | 45 | 7 |
| O60268 | Alpha-2 (XI) collagen chain | 171670 | 61 | 10 |
| O60269 | Alpha-3 (VI) collagen chain | 0 | 37 | 2 |
| O60270 | Cortactin Binding Protein 2 | 0 | 60 | 3 |
| O60271 | Protein 1 containing domain | 388621 | 40 | 3 |
| | CUB and Sushi | | | |
| O60272 | Cubilin | 398480 | 47 | 5 |
| O60273 | Dissociated protein 1 from | 136289 | 174 | 1 |
| | NEDD8 associated with Cullin | | | |
| O60274 | Cystatin-C | 15789 | 54 | 1 |
| O60275 | Heavy chain 1 of dynein 1 | 532072 | 54 | 6 |
| | cytoplasmic | | | |
| O60276 | Protein Cytokinesis Dedicator | 0 | 50 | 3 |
| | 6 | | | |
| O60277 | Dermcidin | 11277 | 66 | 1 |
| O60278 | Desmoplakin | 331569 | 49 | 4 |
| O60279 | Dihydrolipoyl dehydrogenase, | 54143 | 177 | 6 |
| | mitochondrial | | | |
| O60280 | Homologous B of protein 2 | 0 | 32 | 3 |
| | that interacts with Disco | | | |
| O60281 | DNA polymerase theta | 197474 | 35 | 4 |
| O60282 | Catalytic subunit of DNA | 0 | 43 | 5 |
| | polymerase zeta | | | |
| O60283 | DNA-dependent protein kinase | 468788 | 65 | 9 |
| | catalytic subunit | | | |
| O60284 | Member 13 of DnaJ homolog | 0 | 49 | 4 |
| | subfamily | | | |
| O60285 | Coupling protein 6 | 0 | 36 | 2 |
| O60286 | Heavy chain 10 of dieine, | 514512 | 46 | 6 |
| | axonemal | | | |
| O60287 | 11 dyneine heavy chain, | 520711 | 55 | 6 |
| | axonemal | | | |
| O60288 | 2 dyneine heavy chain, | 0 | 35 | 4 |
| | axonemal | | | |
| O60289 | Heavy chain 3 of dynein, | 470468 | 46 | 5 |
| | axonemal | | | |
| 060290 | 5 dyneine heavy chain, | 528684 | 47 | 5 |
| | axonemal | | | |
| O60291 | 6 dyneine heavy chain, | 475679 | 35 | 7 |
| | axonemal | | | |
| O60292 | 8 dyneine heavy chain, | 514335 | 84 | 5 |
| | axonemal | | | |
| O60293 | Protein 1 containing dyneine | 0 | 42 | 5 |
| | heavy chain domain | | | |
| O60294 | Distonin | 0 | 50 | 4 |
| O60295 | Distroglycan | 97381 | 74 | 1 |
| O60296 | Dystrophin | 0 | 34 | 3 |
| O60297 | P400 protein that binds E1A | 343276 | 35 | 7 |
| O60298 | E3 SUMO-protein ligase RanBP2 | 357974 | 68 | 5 |
| O60299 | E3 ubiquitin-protein ligase | 526895 | 55 | 5 |
| | HERC2 | | | |
| O60300 | E3 ubiquitin-protein ligase | 0 | 46 | 4 |
| | HUWE1 | | | |
| O60301 | E3 ubiquitin-protein ligase | 0 | 52 | 2 |
| | UBR4 | | | |
| O60302 | Protein similar to 6 | 0 | 37 | 4 |
| | associated with Echinoderm | | | |
| | microtubule | | | |
| O60303 | Elongation factor-alpha 1 | 50109 | 285 | 4 |
| O60304 | Gamma elongation factor 1 | 50087 | 185 | 1 |
| O60305 | Elongation phantom 2 | 95277 | 498 | 4 |
| O60306 | Eukaryotic initiation | 46125 | 58 | 1 |
| | spectrum 4A-I | | | |
| O60307 | Eukaryotic initiation factor | 46373 | 58 | 1 |
| | 4A-II | | | |
| O60308 | ERF-3B subunit linking | 0 | 32 | 1 |
| | eukaryotic peptide chain GTP | | | |
| | factor | | | |
| O60309 | Exophilin-5 | 0 | 40 | 6 |
| O60310 | FRAS1 extracellular matrix | 442646 | 36 | 6 |
| | protein | | | |
| O60311 | Ezrin | 69370 | 804 | 6 |
| O60312 | Anemia of Fanconi group I | 149229 | 40 | 4 |
| | protein | | | |
| O60313 | Farnesyl pyrophosphate | 48245 | 256 | 2 |
| | synthase | | | |
| O60314 | Fascin | 54496 | 253 | 7 |
| O60315 | Fatty acid synthase | 273254 | 853 | 23 |
| O60316 | Protein 1 containing type III | 0 | 43 | 7 |
| | fibronectin domain | | | |
| O60317 | Protein 2 that interacts with | 780119 | 44 | 6 |
| | fibrous sheath | | | |
| O60318 | Filagrine | 434922 | 45 | 10 |
| O60319 | Filamine-B | 277990 | 90 | 5 |
| O60320 | Fructose bisphosphate | 39395 | 813 | 18 |
| | aldolase A | | | |
| O60321 | Fructose Bisphosphate | 39431 | 190 | 4 |
| | Aldolase C | | | |
| O60322 | Galectin-1 | 14706 | 44 | 1 |
| O60323 | Protein that binds galectin-3 | 65289 | 628 | 17 |
| O60324 | Gamma-enolase | 47239 | 221 | 2 |
| O60325 | Girdin | 0 | 37 | 3 |
| O60326 | Glial fibrillary acidic | 49850 | 81 | 2 |
| | protein | | | |
| O60327 | Glucokinase | 0 | 36 | 3 |
| O60328 | Glucose-6-phosphate isomerase | 63107 | 892 | 6 |
| O60329 | Glutamate receptor 3A subunit | 125385 | 48 | 4 |
| | [NMDA] | | | |
| O60330 | Glutathione S-transferase | 27548 | 76 | 2 |
| | omega-1 | | | |
| O60331 | Glyceraldehyde-3-phosphate | 36030 | 572 | 2 |
| | dehydrogenase | | | |
| O60332 | Alpha chain glycoprotein | 13066 | 47 | 2 |
| | hormones | | | |
| O60333 | 98 G-protein coupled receptor | 0 | 37 | 4 |
| O60334 | GTP-binding nuclear Ran | 24408 | 179 | 4 |
| | protein | | | |
| O60335 | Subunit of beta-2 protein | 35055 | 32 | 2 |
| | similar to 1 nucleotide | | | |
| | binding of Guanine | | | |
| O60336 | 70 kDa 1A/1B protein from | 70009 | 639 | 2 |
| | thermal shock | | | |
| O60337 | Protein 1 of 70 kDa of | 70331 | 345 | 1 |
| | similar thermal shock | | | |
| O60338 | Protein 6 of 70 kDa of | 70984 | 253 | 2 |
| | thermal shock | | | |
| O60339 | 71 kDa protein with thermal | 70854 | 661 | 1 |
| | shock | | | |
| O60340 | Beta-1 thermal shock protein | 22768 | 79 | 4 |
| O60341 | HSP 90-alpha thermal shock | 84607 | 874 | 12 |
| | protein | | | |
| O60342 | HSP 90-beta thermal shock | 83212 | 1010 | 10 |
| | protein | | | |
| O60343 | Hemicentin-1 | 613001 | 42 | 6 |
| O60344 | Hemicentin-2 | 542265 | 47 | 5 |
| O60345 | Hemoglobin alpha subunit | 15248 | 56 | 2 |
| O60346 | Beta subunit of hemoglobin | 15988 | 38 | 1 |
| O60347 | Hepatoma-derived growth | 26772 | 63 | 2 |
| | factor | | | |
| O60348 | heterogenic nuclear | 0 | 46 | 1 |
| | ribonucleoprotein A0 | | | |
| O60349 | heterologous nuclear A1 | 38723 | 113 | 1 |
| | ribonucleoprotein | | | |
| O60350 | ribonucleoprotein A1-similar | 34204 | 94 | 1 |
| | to 2 nuclear heterogeneous | | | |
| O60351 | heterogeneous nuclear | 39571 | 57 | 1 |
| | ribonucleoprotein A3 | | | |
| O60352 | heterogeneous nuclear D0 | 38410 | 114 | 3 |
| | ribonucleoprotein | | | |
| O60353 | heterogeneous nuclear A2/B1 | 37407 | 217 | 3 |
| | ribonucleoproteins | | | |
| O60354 | Histone H2A type 1 | 14083 | 134 | 1 |
| O60355 | Histone H2A type 1-B/E | 14127 | 134 | 1 |
| O60356 | Histone H2A type 1-C | 14097 | 134 | 1 |
| O60357 | Histone H2A type 1-D | 14099 | 134 | 1 |
| O60358 | Histone H2A type 1-H | 13898 | 134 | 1 |
| O60359 | Histone H2A type 1-J | 13928 | 134 | 1 |
| O60360 | Histone H2A type 2-A | 14087 | 134 | 1 |
| O60361 | Histone H2A type 2-B | 13987 | 58 | 1 |
| O60362 | Histone H2A type 2-C | 13980 | 134 | 1 |
| O60363 | Histone H2A type 3 | 14113 | 134 | 1 |
| O60364 | Histone H2A.J | 14011 | 134 | 1 |
| O60365 | Histone H2A.x | 15135 | 58 | 1 |
| O60366 | Histone H2B type 1-H | 13884 | 100 | 4 |
| O60367 | Histone H2B type 2-F | 13912 | 100 | 4 |
| O60368 | Histone H3.1 | 15394 | 158 | 6 |
| O60369 | Histone H3.1t | 15499 | 155 | 6 |
| O60370 | Histone H3.2 | 15379 | 158 | 6 |
| O60371 | Histone H3.3 | 15318 | 160 | 6 |
| O60372 | Histone H3.3C | 15204 | 44 | 1 |
| O60373 | Histone H4 | 11360 | 178 | 7 |
| O60374 | Histone-lysine n- | 593017 | 49 | 6 |
| | methyltransferase MLL2 | | | |
| O60375 | Histone-lysine N- | 0 | 42 | 6 |
| | methyltransferase MLL3 | | | |
| O60376 | Histone-lysine N- | 0 | 39 | 4 |
| | methyltransferase SETD1A | | | |
| O60377 | Histone-lysine N- | 0 | 42 | 5 |
| | methyltransferase, H3 lysine- | | | |
| | 36 and H4 lysine-20 specific | | | |
| O60378 | Cutting protein-similar to 1 | 0 | 38 | 3 |
| | homeosequence | | | |
| O60379 | Protein homolog that induces | 0 | 60 | 4 |
| | hydrocephalus | | | |
| O60380 | Protein that binds IgGFc | 571639 | 40 | 3 |
| O60381 | Above that degrades insulin | 0 | 36 | 3 |
| O60382 | Integrin alpha-10 | 0 | 32 | 4 |
| O60383 | Factor 3 linking interleukin | 95279 | 48 | 3 |
| | enhancer | | | |
| O60384 | Intraflagelar transport | 0 | 43 | 4 |
| | protein 81 counterpart | | | |
| O60385 | Keratin, type I cuticular Ha5 | 0 | 34 | 2 |
| O60386 | Keratin, type I cytoskeletal | 58792 | 962 | 11 |
| | 10 | | | |
| O60387 | Keratin, type I cytoskeletal | 53478 | 75 | 2 |
| | 12 | | | |
| O60388 | Keratin, type I cytoskeletal | 49557 | 91 | 3 |
| | 13 | | | |
| O60389 | Keratin, type I cytoskeletal | 51529 | 252 | 4 |
| | 14 | | | |
| O60390 | Keratin, type I cytoskeletal | 49181 | 114 | 3 |
| | 15 | | | |
| O60391 | Keratin, type I cytoskeletal | 51236 | 184 | 4 |
| | 16 | | | |
| O60392 | Keratin, type I cytoskeletal | 48076 | 123 | 4 |
| | 17 | | | |
| O60393 | Keratin, type I cytoskeletal | 48029 | 103 | 3 |
| | 18 | | | |
| O60394 | Keratin, type I cytoskeletal | 44079 | 70 | 2 |
| | 19 | | | |
| O60395 | Keratin, type I cytoskeletal | 49287 | 73 | 3 |
| | 25 | | | |
| O60396 | Keratin, type I cytoskeletal | 49792 | 68 | 3 |
| | 27 | | | |
| O60397 | Keratin, type I cytoskeletal | 50536 | 149 | 4 |
| | 28 | | | |
| O60398 | Keratin, type I cytoskeletal | 62027 | 1295 | 12 |
| | 9 | | | |
| O60399 | Keratin, type II cuticular | 64801 | 85 | 3 |
| | Hb4 | | | |
| O60400 | Keratin, type II cytoskeletal | 65999 | 1475 | 16 |
| | 1 | | | |
| O60401 | Keratin, type II cytoskeletal | 61864 | 217 | 3 |
| | 1b | | | |
| O60402 | Keratin, type II epidermal | 65393 | 1547 | 13 |
| | cytoskeletal 2 | | | |
| O60403 | Keratin, type II oral | 65800 | 185 | 4 |
| | cytoskeletal 2 | | | |
| O60404 | Keratin, type II cytoskeletal | 64378 | 177 | 3 |
| | 3 | | | |
| O60405 | Keratin, type II cytoskeletal | 57250 | 195 | 4 |
| | 4 | | | |
| O60406 | Keratin, type II cytoskeletal | 62340 | 352 | 6 |
| | 5 | | | |
| O60407 | Keratin, type II cytoskeletal | 60008 | 300 | 5 |
| | 6A | | | |
| O60408 | Keratin, type II cytoskeletal | 60030 | 330 | 4 |
| | 6B | | | |
| O60409 | Keratin, type II cytoskeletal | 51354 | 124 | 3 |
| | 7 | | | |
| O60410 | Keratin, type II cytoskeletal | 57256 | 101 | 4 |
| | 71 | | | |
| O60411 | Keratin, type II cytoskeletal | 55842 | 97 | 3 |
| | 72 | | | |
| O60412 | Keratin, type II cytoskeletal | 58887 | 157 | 4 |
| | 73 | | | |
| O60413 | Keratin, type II cytoskeletal | 57830 | 118 | 4 |
| | 74 | | | |
| O60414 | Keratin, type II cytoskeletal | 59524 | 140 | 3 |
| | 75 | | | |
| O60415 | Keratin, type II cytoskeletal | 0 | 42 | 3 |
| | 78 | | | |
| O60416 | Keratin, type II cytoskeletal | 57800 | 181 | 4 |
| | 79 | | | |
| O60417 | Keratin, type II cytoskeletal | 53671 | 131 | 4 |
| | 8 | | | |
| O60418 | Keratin, type II cytoskeletal | 50494 | 78 | 2 |
| | 80 | | | |
| O60419 | Close relationship of IRRE- | 0 | 46 | 3 |
| | similar to protein 2 | | | |
| O60420 | Kinesin-like protein KIF17 | 0 | 34 | 2 |
| O60421 | Kinesin-like protein KIF18B | 0 | 35 | 4 |
| O60422 | Kinesin-like protein KIF20B | 0 | 58 | 2 |
| O60423 | Kinesin-like protein KIF26A | 194468 | 47 | 2 |
| O60424 | Laminin alpha-1 subunit | 336867 | 34 | 5 |
| O60425 | Laminin alpha-5 subunit | 0 | 32 | 5 |
| O60426 | Laminin gamma-1 subunit | 177489 | 35 | 2 |
| O60427 | Lethal malignant brain tumor | 0 | 38 | 4 |
| | (3)-similar to protein 3 | | | |
| O60428 | Protein kinase 2 | 0 | 48 | 3 |
| | serine/threonine rich in | | | |
| | leucine repetition | | | |
| O60429 | 16A protein containing | 151462 | 44 | 5 |
| | leucine-rich repeats | | | |
| O60430 | Receptive lipopolysaccharide | 0 | 34 | 5 |
| | and anchor protein similar to | | | |
| | beige | | | |
| O60431 | L-lactate dehydrogenase A | 36665 | 534 | 4 |
| | chain | | | |
| O60432 | L-lactate dehydrogenase A- | 36484 | 55 | 2 |
| | similar to 6A | | | |
| O60433 | L-lactate dehydrogenase A- | 41916 | 120 | 4 |
| | similar to 6B | | | |
| O60434 | L-lactate dehydrogenase B | 36615 | 552 | 3 |
| | chain | | | |
| O60435 | 1B protein related to low | 515159 | 45 | 7 |
| | density lipoprotein receptor | | | |
| O60436 | Protein 4 related to low | 0 | 38 | 5 |
| | density lipoprotein receptor | | | |
| O60437 | Protein 6 related to low | 180314 | 39 | 3 |
| | density lipoprotein receptor | | | |
| O60438 | Lysine specific demethylase | 175545 | 37 | 3 |
| | 5B | | | |
| O60439 | Lysine specific histone | 92039 | 37 | 5 |
| | demethylase 1B | | | |
| O60440 | Malate dehydrogenase, | 36403 | 351 | 5 |
| | cytoplasmic | | | |
| O60441 | Malate dehydrogenase, | 35481 | 113 | 3 |
| | mitochondrial | | | |
| O60442 | Protein associated with gene | 0 | 47 | 6 |
| | MAX | | | |
| O60443 | Metalloproteinase Inhibitor 1 | 23156 | 90 | 2 |
| O60444 | Methylcytosine dioxygenase | 0 | 49 | 4 |
| | TET1 | | | |
| O60445 | Factor 1 reticulating | 669721 | 50 | 7 |
| | microtubule, isoform 4 | | | |
| O60446 | Microtubule-actin | 0 | 44 | 5 |
| | crosslinking factor 1, | | | |
| | 1/2/3/5 isoforms | | | |
| O60447 | Microtubule-associated | 143049 | 49 | 5 |
| | serine/threonine protein | | | |
| | kinase 3 | | | |
| O60448 | Suppressor tumor candidate 2 | 0 | 53 | 5 |
| | associated with microtubule | | | |
| O60449 | Midasin | 0 | 62 | 10 |
| O60450 | Moesin | 67778 | 719 | 6 |
| O60451 | Mucin-16 | 0 | 59 | 5 |
| O60452 | Mucin-19 | 597790 | 37 | 4 |
| O60453 | Multiple epidermal growth | 161072 | 38 | 5 |
| | factor-similar to protein 6 | | | |
| | domains | | | |
| O60454 | Protein 2 associated with | 29394 | 36 | 3 |
| | multiple myeloma tumor | | | |
| O60455 | Miosin-14 | 227863 | 38 | 2 |
| O60456 | Miosin-3 | 223766 | 39 | 4 |
| O60457 | Miosin-7B | 221251 | 41 | 6 |
| O60458 | Miosin-XV | 395044 | 41 | 6 |
| O60459 | Nck associated protein 5 | 208409 | 41 | 6 |
| O60460 | Nebulin | 0 | 44 | 5 |
| O60461 | Nesprin-1 | 1010398 | 39 | 8 |
| O60462 | Neurobeachin-similar to | 0 | 39 | 4 |
| | protein 2 | | | |
| O60463 | AHNAK protein associated with | 628699 | 76 | 9 |
| | neuroblast differentiation | | | |
| O60464 | Neurofibromin | 0 | 37 | 4 |
| O60465 | Notch protein 3 counterpart | 0 | 37 | 4 |
| | of neurogenic site | | | |
| O60466 | Neuron browser 1 | 0 | 43 | 4 |
| O60467 | Pentraxin-1 neuronal | 47093 | 240 | 10 |
| O60468 | Protein 1 of nuclear mitotic | 238115 | 81 | 6 |
| | apparatus | | | |
| O60469 | Nucleoside diphosphate kinase | 17138 | 348 | 9 |
| | A | | | |
| O60470 | Nucleoside diphosphate kinase | 17287 | 284 | 7 |
| | B | | | |
| O60471 | BPTF subunit factor | 338054 | 36 | 5 |
| | remodeling nucleosome | | | |
| O60472 | Obg-similar to ATPase 1 | 44715 | 118 | 1 |
| O60473 | Obscurin | 867940 | 45 | 6 |
| O60474 | Defective partition 6 gamma | 0 | 33 | 3 |
| | homologue | | | |
| O60475 | Peptidyl-prolyl cis-trans | 18001 | 285 | 3 |
| | isomerase A | | | |
| O60476 | Peptidyl-prolyl cis-trans | 51772 | 278 | 7 |
| | isomerase FKBP4 | | | |
| O60477 | Pericentrin | 0 | 39 | 6 |
| O60478 | Peroxidasin Homolog | 0 | 34 | 4 |
| O60479 | Peroxiredoxin-1 | 22096 | 317 | 2 |
| O60480 | Peroxiredoxin-2 | 21878 | 378 | 8 |
| O60481 | Peroxiredoxin-4 | 30521 | 205 | 5 |
| O60482 | Peroxiredoxin-6 | 25019 | 237 | 5 |
| O60483 | Protein 1 that binds | 21044 | 252 | 4 |
| | phosphatidylethanolamine | | | |
| O60484 | Beta subunit containing | 0 | 40 | 3 |
| | phosphatidylinositol-4- | | | |
| | phosphate 3-kinase C2 domain | | | |
| O60485 | Phosphoglucomutase-1 | 61411 | 240 | 6 |
| O60486 | Phosphoglycerate kinase 1 | 44586 | 668 | 1 |
| O60487 | Phosphoglycerate mutase 1 | 28786 | 135 | 5 |
| O60488 | Phosphoserine | 40397 | 128 | 3 |
| | aminotransferase | | | |
| O60489 | Member 2 of family G containing | 147877 | 39 | 4 |
| | Pleckstrin homology domain | | | |
| O60490 | Plectin | 531466 | 93 | 5 |
| O60491 | Plexin-A1 | 210933 | 44 | 4 |
| O60492 | 1-similar protein to 1 of | 0 | 55 | 4 |
| | polycystic kidney disease | | | |
| O60493 | Polycystin-1 | 0 | 53 | 5 |
| O60494 | Polyubiquitin-B | 25746 | 158 | 3 |
| O60495 | Polyubiquitin-C | 76982 | 148 | 3 |
| O60496 | Family member E of domain | 121286 | 444 | 4 |
| | POTE anchirine | | | |
| O60497 | Prelamin-A/C | 74095 | 123 | 2 |
| O60498 | Likely ATP DDX41-dependent | 0 | 35 | 3 |
| | RNA helicase | | | |
| O60499 | Likely ATP-dependent DDX60- | 0 | 35 | 2 |
| | similar RNA helicase | | | |
| O60500 | Likely ubiquitin-similar E3 | 0 | 37 | 3 |
| | HERC1 ligase protein | | | |
| O60501 | Likely MYCBP2 protein ligase | 509759 | 60 | 5 |
| | from E3 ubiquitin | | | |
| O60502 | Likely terminal hydrolase | 0 | 33 | 4 |
| | FAF-Y of terminal ubiquitin | | | |
| | carboxyl | | | |
| O60503 | Profilin-1 | 15045 | 179 | 4 |
| O60504 | Protein 1 related to density | 504276 | 46 | 9 |
| | tested lipoprotein receptor | | | |
| O60505 | Proprotein convertase | 74239 | 340 | 5 |
| | subtilisin/kexin type 9 | | | |
| O60506 | Proteasome subunit alpha | 29537 | 188 | 7 |
| | type-1 | | | |
| O60507 | Proteasome subunit alpha | 25882 | 178 | 6 |
| | type-2 | | | |
| O60508 | Proteasome subunit alpha | 28415 | 196 | 5 |
| | type-3 | | | |
| O60509 | Proteasome subunit alpha | 27382 | 303 | 8 |
| | type-6 | | | |
| O60510 | Proteasome subunit alpha | 27870 | 464 | 9 |
| | type-7 | | | |
| O60511 | Proteasome beta subunit type- | 26472 | 125 | 3 |
| | 1 | | | |
| O60512 | Proteasome subunit beta type- | 22933 | 150 | 5 |
| | 3 | | | |
| O60513 | Proteasome beta subunit type- | 29185 | 93 | 3 |
| | 4 | | | |
| O60514 | Proteasome subunit beta type- | 28462 | 355 | 7 |
| | 5 | | | |
| O60515 | Proteasome subunit beta type- | 25341 | 155 | 3 |
| | 6 | | | |
| O60516 | AHNAK2 protein | 0 | 38 | 5 |
| O60517 | Arginine N-methyltransferase | 0 | 59 | 1 |
| | 3 protein | | | |
| O60518 | Bassoon protein | 416214 | 46 | 3 |
| O60519 | Daple protein | 228091 | 37 | 5 |
| O60520 | Disulfide isomerase protein | 57081 | 98 | 5 |
| O60521 | Disulfide-isomerase A3 | 56747 | 635 | 13 |
| | protein | | | |
| O60522 | A6 disulfide isomerase protein | 48091 | 172 | 3 |
| O60523 | FAM179A protein | 111084 | 33 | 3 |
| O60524 | Protein counterpart “bassoon” | 0 | 34 | 6 |
| O60525 | Irregular protein-2 | 133277 | 42 | 2 |
| O60526 | “Flutin” protein | 566309 | 44 | 4 |
| O60527 | Shroom2 protein | 176302 | 34 | 6 |
| O60528 | Shroom3 protein | 216724 | 45 | 1 |
| O60529 | Companion protein-1 | 0 | 34 | 3 |
| O60530 | SZT2 protein | 0 | 38 | 6 |
| O60531 | TANC1 protein | 0 | 46 | 5 |
| O60532 | Protein-arginine deiminase | 74618 | 42 | 1 |
| | type-1 | | | |
| O60533 | Protocadherina Fat 1 | 0 | 33 | 6 |
| O60534 | Protocadherina Fat 3 | 505209 | 35 | 4 |
| O60535 | Purine nucleoside | 32097 | 127 | 4 |
| | phosphorylase | | | |
| O60536 | Putative beta-actin-like | 41989 | 230 | 5 |
| | protein 3 | | | |
| O60537 | Putative elongation factor 1- | 50153 | 285 | 4 |
| | alpha-similar to 3 | | | |
| O60538 | HSP 90-beta 2 protein from | 44321 | 237 | 3 |
| | putative thermal shock | | | |
| O60539 | Putative heterogeneous | 34202 | 113 | 1 |
| | nuclear A1 ribonucleoprotein- | | | |
| | similar to 3 | | | |
| O60540 | ASXL3 protein from the | 241767 | 37 | 4 |
| | putative Polycomb group | | | |
| O60541 | Putative tubulin similar to | 27534 | 39 | 2 |
| | alpha-4B protein | | | |
| O60542 | Isozymes M1/M2 pyruvate | 57900 | 661 | 1 |
| | kinase isozymes M1/M2 | | | |
| O60543 | Rab GDP alpha dissociation | 50550 | 44 | 2 |
| | inhibitor | | | |
| O60544 | Rab GDP dissociation beta inhibitor | 50631 | 549 | 3 |
| O60545 | Radixin | 68521 | 524 | 3 |
| O60546 | Ras-related Rab-28 protein | 0 | 37 | 1 |
| O60547 | Tyrosine-protein phosphatase | 224161 | 35 | 3 |
| | beta receptor-type | | | |
| O60548 | Regulation of protein 1 of | 188956 | 38 | 6 |
| | synaptic exocytosis | | | |
| O60549 | Retinitis pigmentosa 1- | 0 | 43 | 3 |
| | similar to protein 1 | | | |
| O60550 | Protein 23 that activates Rho | 0 | 37 | 6 |
| | GTPase | | | |
| O60551 | Factor 2 homolog of Ribosome | 35560 | 41 | 4 |
| | production | | | |
| O60552 | Ribosome binding protein 1 | 0 | 47 | 6 |
| O60553 | Rootletin | 228388 | 49 | 7 |
| O60554 | Ryanodine receptor 2 | 564206 | 52 | 7 |
| O60555 | Sacsin | 0 | 48 | 7 |
| O60556 | Splicing factor 1 rich in | 27728 | 47 | 2 |
| | serine/arginine | | | |
| O60557 | Serine/threonine-protein | 143903 | 34 | 3 |
| | kinase 36 | | | |
| O60558 | Serine/threonine-protein | 0 | 35 | 4 |
| | kinase ATR | | | |
| O60559 | Serine/threonine-protein | 0 | 44 | 5 |
| | kinase SMG1 | | | |
| O60560 | Serine/threonine-protein | 0 | 50 | 4 |
| | kinase WNK2 | | | |
| O60561 | Serpin B6 | 42594 | 160 | 4 |
| O60562 | Serpin H1 | 46411 | 145 | 5 |
| O60563 | Serum albumin | 69321 | 267 | 4 |
| O60564 | SH3 and multiple protein | 0 | 43 | 3 |
| | domain 2 repeat ankyrins | | | |
| O60565 | SH3 and multiple protein 3 | 0 | 44 | 6 |
| | domain repeat ankyrins | | | |
| O60566 | Signal transducer and | 0 | 46 | 3 |
| | transcription activator 2 | | | |
| O60567 | Spectrin alpha chain, brain | 284364 | 343 | 4 |
| O60568 | Spectrin beta chain, brain 3 | 0 | 125 | 3 |
| O60569 | Spectrin beta chain, brain 4 | 0 | 39 | 5 |
| O60570 | Spliceosoma RNA helicase BAT1 | 48960 | 51 | 2 |
| O60571 | Stabilin-1 | 275300 | 36 | 5 |
| O60572 | Protein 1 containing - domain- | 0 | 55 | 2 |
| | like - Sushi, nidogen and EGF. | | | |
| O60573 | Protein 1 containing Sushi, | 0 | 39 | 5 |
| | von Willebrand factor type A, | | | |
| | EGF and pentraxin domain | | | |
| O60574 | Protein targeting Xklp2 | 0 | 33 | 2 |
| O60575 | Component 1 of telomerase | 290307 | 36 | 4 |
| | protein | | | |
| O60576 | Tenascin-X | 0 | 36 | 6 |
| O60577 | Tensin-1 | 185586 | 69 | 2 |
| O60578 | Thioredoxin | 11730 | 187 | 1 |
| O60579 | Thioredoxin reductase 1, | 70711 | 392 | 10 |
| | cytoplasmic | | | |
| O60580 | Titin | 3813810 | 124 | 24 |
| O60581 | Toll-similar to receptor 10 | 0 | 40 | 2 |
| O60582 | C10orf93 protein containing | 0 | 34 | 4 |
| | TPR repeat | | | |
| O60583 | Transcription factor TFIIIB | 293705 | 35 | 7 |
| | component B “homologous | | | |
| O60584 | Transferrin receptor protein 1 | 84818 | 896 | 4 |
| O60585 | Protein associated with | 437318 | 52 | 7 |
| | domain | | | |
| | transformation/transcription | | | |
| O60586 | Protein 2 containing acidic | 309237 | 36 | 6 |
| | double helix transformation | | | |
| O60587 | ATPase transitional | 89266 | 338 | 1 |
| | endoplasmic reticulum | | | |
| O60588 | Transketolase | 67835 | 965 | 5 |
| O60589 | Treacle protein | 0 | 42 | 5 |
| O60590 | Triosephosphate isomerase | 26653 | 703 | 16 |
| O60591 | Triple functional domain | 0 | 51 | 4 |
| | protein | | | |
| O60592 | Alpha-1B tubulin chain | 50120 | 715 | 15 |
| O60593 | Alpha-1C tubulin chain | 49863 | 715 | 15 |
| O60594 | Alpha-4A tubulin chain | 49892 | 39 | 2 |
| O60595 | Alpha-8 tubulin chain | 50062 | 39 | 2 |
| O60596 | Beta tubulin chain | 49639 | 442 | 9 |
| O60597 | Beta-3 tubulin chain | 50400 | 245 | 5 |
| O60598 | Beta-8 tubulin chain | 49744 | 102 | 3 |
| O60599 | Ribosomal ubiquitin-40S S27a | 17953 | 167 | 3 |
| | protein | | | |
| O60600 | L40 protein from ribosomal | 14719 | 167 | 3 |
| | ubiquitin-60S | | | |
| O60601 | Uncharacterized C4orf37 | 50628 | 32 | 3 |
| | protein | | | |
| O60602 | KIAA0802 protein without | 0 | 50 | 6 |
| | characterization | | | |
| O60603 | KIAA1109 protein without | 0 | 59 | 6 |
| | characterization | | | |
| O60604 | KIAA1614 protein without | 0 | 40 | 6 |
| | characterization | | | |
| O60605 | UPF0556 C19orf10 protein | 18783 | 218 | 7 |
| O60606 | Urotensin-2 | 0 | 34 | 1 |
| O60607 | Utrophin | 394220 | 38 | 4 |
| O60608 | 13D protein associated with | 491535 | 44 | 4 |
| | vacuolar protein | | | |
| O60609 | Vinculin | 123722 | 358 | 9 |
| O60610 | Von Willebrand factor | 309058 | 38 | 5 |
| O60611 | Protein 3 containing WD | 0 | 38 | 5 |
| | repeat and FYVE domain | | | |
| O60612 | Protein 35 containing WD | 0 | 36 | 2 |
| | repeating | | | |
| O60613 | KIAA1875 protein containing | 180192 | 38 | 4 |
| | WD repeat | | | |
| O60614 | Protein 2 containing Xin | 0 | 36 | 5 |
| | Actin binding repeater | | | |
| O60615 | Protein 13 containing CCCH | 0 | 44 | 6 |
| | zinc finger domain | | | |
| O60616 | Zinc finger protein 142 | 187758 | 34 | 3 |
| O60617 | Zinc finger 469 protein | 409949 | 44 | 5 |

TABLE 5
Proteins secreted in SiHa cells.

| | | | | Matching |
|---|---|---|---|---|
| ID | Name | Mass | Score | peptides |
| P31946 | 14-3-3 beta/alpha protein | 28307 | 78 | 3 |
| P62258 | 14-3-3 epsilon protein | 29486 | 287 | 5 |
| P27348 | 14-3-3 theta protein | 28128 | 75 | 3 |
| P63104 | 14-3-3 zeta/delta protein | 28011 | 142 | 4 |
| Q9NQ66 | 1-phosphatidylinositol-4,5- | 0 | 44 | 3 |
| | bisphosphate | | | |
| | phosphodiesterase beta-1 | | | |
| Q4KWH8 | 1-phosphatictylinositol-4,5- | 0 | 39 | 2 |
| | bisphosphate phosphodiesterase eta-1 | | | |
| P43686 | 26B Protease Regulatory 6B | 47644 | 96 | 3 |
| | Subunit | | | |
| P62269 | S18 40S ribosomal protein | 17740 | 73 | 3 |
| P62847 | S24 40S ribosomal protein | 15509 | 61 | 1 |
| P62081 | S7 40S ribosomal protein | 22145 | 119 | 4 |
| P08865 | SA 40S ribosomal protein | 0 | 37 | 4 |
| P32754 | 4-hydroxyphenylpyruvate | 45256 | 313 | 7 |
| | dioxygenase | | | |
| P10809 | 60 kDa thermal shock | 61478 | 288 | 8 |
| | protein, mitochondrial | | | |
| P05388 | P0 60S acidic ribosomal | 34567 | 229 | 5 |
| | protein | | | |
| P62913 | L11 60S ribosomal protein | 20516 | 114 | 2 |
| P30050 | L12 60S ribosomal protein | 18011 | 51 | 1 |
| P83731 | L24 60S ribosomal protein | 17930 | 46 | 1 |
| P52209 | 6-phosphogluconate | 53852 | 257 | 4 |
| | dehydrogenase, | | | |
| | decarboxylation | | | |
| P11021 | 78 kDa regulated glucose | 72580 | 410 | 9 |
| | protein | | | |
| Q8TE58 | A disintegrine and | 106076 | 37 | 1 |
| | metalloproteinase with | | | |
| | thrombospondin motifs 15 | | | |
| Q8IZT6 | Associated protein-similar | 414501 | 44 | 6 |
| | to abnormal microcephaly- | | | |
| | spindle | | | |
| Q9BWD1 | Acetyl-CoA | 42030 | 70 | 2 |
| | acetyltransferase, cytosolic | | | |
| P68032 | Actin, heart muscle 1 alpha | 42596 | 280 | 12 |
| P68133 | Actin, alpha skeletal muscle | 42644 | 280 | 12 |
| P62736 | Actin, aortic smooth muscle | 42644 | 108 | 2 |
| P60709 | Actin, cytoplasmic 1 | 42330 | 721 | 19 |
| P63261 | Actin, cytoplasmic 2 | 42386 | 721 | 19 |
| P53999 | Transcriptional p15 co- | 14466 | 41 | 1 |
| | activator of activated RNA | | | |
| | polymerase | | | |
| P23526 | Adenosylhomocysteinase | 48521 | 188 | 5 |
| P00568 | Adenylate kinase isoenzyme 1 | 21815 | 69 | 2 |
| Q01518 | Protein 1 associated with | 52493 | 62 | 2 |
| | adenylyl cyclase | | | |
| P84077 | ADP ribosylation factor 1 | 20838 | 48 | 2 |
| P61204 | ADP-ribosylation factor 3 | 20742 | 48 | 2 |
| P84085 | ADP-ribosylation factor 5 | 20727 | 48 | 1 |
| P12814 | alpha-actinin-1 | 104005 | 340 | 12 |
| O43707 | alpha-actinin-4 | 105636 | 483 | 15 |
| P06733 | alpha-enolase | 47631 | 885 | 10 |
| Q8TCU4 | Alstrom syndrome protein 1 | 463685 | 50 | 5 |
| Q9BXX3 | Protein 30A containing | 161035 | 43 | 5 |
| | Ankyrin repeat domain | | | |
| Q53LP3 | Protein 57 containing repeat | 56003 | 36 | 3 |
| | domain of Ankyrin | | | |
| Q12955 | Ankyrin-3 | 483809 | 43 | 6 |
| P07355 | Annex A2 | 38952 | 330 | 6 |
| P08758 | Annex A5 | 36099 | 145 | 3 |
| P46013 | KI-67 antigen | 361481 | 62 | 5 |
| P04114 | Apolipoprotein B-100 | 517939 | 64 | 6 |
| O43150 | Arf-GAP with SH3 domain, | 0 | 40 | 3 |
| | protein 2 containing PH | | | |
| | domain and ANK repeat | | | |
| P00966 | Argininosuccinate synthase | 46935 | 310 | 10 |
| P54136 | Arginyl-tRNA synthetase, cytoplasmic | 76415 | 33 | 3 |
| P15848 | Arilsulfatase B | 0 | 36 | 2 |
| P17174 | Aspartate aminotransferase, | 46543 | 111 | 4 |
| | cytoplasmic | | | |
| Q7Z591 | Transcription factor | 156176 | 56 | 4 |
| | containing AT hitch | | | |
| Q5T9A4 | 3B protein containing AAA | 0 | 41 | 5 |
| | domain of the ATPase family | | | |
| Q96QE3 | Protein 5 containing AAA | 209929 | 35 | 3 |
| | domain of the ATPase family | | | |
| Q86UQ4 | Member 13 of cassette | 582507 | 46 | 4 |
| | subfamily A linking ATP | | | |
| Q8IZY2 | Member 7 of cassette | 236929 | 40 | 3 |
| | subfamily A linking ATP | | | |
| P53396 | ATP-citrate synthase | 122234 | 657 | 3 |
| Q03989 | 5A protein containing | 0 | 39 | 2 |
| | interactive domain rich in | | | |
| | AT | | | |
| P98160 | Base heparan sulfate | 479940 | 37 | 3 |
| | proteoglycan basement | | | |
| | membrane specific protein | | | |
| Q5H9F3 | Co-repressor BCL-6-similar | 0 | 33 | 4 |
| | to protein 1 | | | |
| Q13884 | Beta-1-Sintrophin | 0 | 35 | 3 |
| Q562R1 | Beta-actin-like protein 2 | 42596 | 156 | 4 |
| P13929 | Beta-enolase | 47394 | 192 | 3 |
| Q8NFC6 | Biorientation of chromosomes | 0 | 35 | 4 |
| | in protein 1 of cell-similar | | | |
| | division | | | |
| O60241 | Brain specific angiogenesis | 0 | 52 | 7 |
| | inhibitor 2 | | | |
| Q9NYQ6 | Receiver 1 type G seven | 335176 | 71 | 5 |
| | steps of EGF LAG chain | | | |
| Q9NYQ7 | Receiver 3 type G seven | 363521 | 32 | 4 |
| | steps of EGF LAG chain | | | |
| Q9H251 | Caderin-23 | 0 | 39 | 6 |
| Q8N3K9 | Protein 5 associated with | 451703 | 43 | 4 |
| | cardiomyopathy | | | |
| Q8WXD9 | Caskin-1 | 0 | 33 | 4 |
| Q9UBR2 | Cathepsin Z | 34610 | 136 | 3 |
| Q14004 | Protein kinase 13 cell | 0 | 38 | 3 |
| | division | | | |
| Q9HC77 | Centromer J protein | 0 | 34 | 4 |
| Q02224 | Centromere associated | 0 | 36 | 3 |
| | protein E | | | |
| Q5VT06 | 350 protein associated with | 0 | 37 | 4 |
| | centrosome | | | |
| O00299 | Chloride intracellular | 27280 | 136 | 3 |
| | channel protein 1 | | | |
| Q12873 | Chromodomain-helicase-DNA | 0 | 37 | 6 |
| | binding protein 3 | | | |
| Q00610 | 1 heavy clatrin chain | 193946 | 143 | 10 |
| Q7Z460 | CLIP association protein 1 | 0 | 35 | 4 |
| P10909 | Clusterin | 53287 | 115 | 2 |
| Q9UBF2 | Coatomer subunit gamma-2 | 0 | 32 | 4 |
| P23528 | Cofilin-1 | 18783 | 245 | 4 |
| Q02388 | Alpha-1 collagen chain (VII) | 296298 | 46 | 4 |
| Q99715 | Alpha-1 collagen chain (XII) | 334878 | 493 | 26 |
| P39060 | Alpha-1 collagen chain | 0 | 42 | 5 |
| | (XVIII) | | | |
| P08123 | Alpha-2 collagen chain (I) | 129917 | 34 | 4 |
| P13942 | Alpha-2 collagen chain (XI) | 0 | 60 | 4 |
| P12111 | Alpha-3 collagen chain (VI) | 345759 | 42 | 6 |
| Q8WZ74 | Cortosterin Binding Protein | 183844 | 37 | 3 |
| P12277 | Creatine kinase type B | 43083 | 39 | 2 |
| Q86VP6 | Dissociated protein 1 from | 138525 | 51 | 2 |
| | NEDD8 associated with cullin | | | |
| P01034 | Cystatin-C | 16081 | 128 | 2 |
| Q14204 | Heavy chain 1 of | 536760 | 48 | 6 |
| | cytoplasmic dinneine 1 | | | |
| Q96HP0 | Protein Cytokinesis | 0 | 40 | 4 |
| | Dedicator 6 | | | |
| O43598 | Deoxyribonucleoside 5′-N- | 19259 | 65 | 1 |
| | glycosidase monophosphate | | | |
| O14531 | Protein 4 related to | 0 | 41 | 4 |
| | dihydropyrimidinase | | | |
| Q14689 | Protein 2 homologue A | 172887 | 41 | 5 |
| | that interacts with Disco | | | |
| O60673 | DNA polymerase zeta | 0 | 32 | 3 |
| | catalytic subunit | | | |
| Q02880 | Topoisomerase 2-beta DNA | 184682 | 33 | 3 |
| P27695 | (apurinic or apirimidinic | 35979 | 50 | 2 |
| | site) DNA lyase | | | |
| O14802 | RPC1 subunit of RNA polymerase | 0 | 36 | 2 |
| | III directed at DNA | | | |
| O75165 | Member 13 of DnaJ homolog | 0 | 35 | 3 |
| | subfamily | | | |
| Q6PKX4 | Coupling protein 6 | 0 | 42 | 5 |
| Q8IVF4 | 10 dyneine heavy chain, | 0 | 48 | 4 |
| | axonemal | | | |
| Q96DT5 | 11 dyneine heavy chain, | 0 | 38 | 7 |
| | axonemal | | | |
| Q9UFH2 | Heavy chain 17 of dynein, | 517327 | 42 | 5 |
| | axonemal | | | |
| Q9P225 | 2 dyneine heavy chain, | 0 | 37 | 6 |
| | axonemal | | | |
| Q8TD57 | Heavy chain 3 dynein, | 476111 | 48 | 5 |
| | axonemal | | | |
| Q8TE73 | 5 dyneine heavy chain, | 0 | 42 | 5 |
| | axonemal | | | |
| Q96M86 | Protein 1 containing | 540950 | 44 | 4 |
| | dinein heavy chain domain | | | |
| Q03001 | Dystonia | 867875 | 62 | 10 |
| Q7Z6Z7 | E3 ubiquitin-protein | 0 | 51 | 4 |
| | ligase HUWE1 | | | |
| Q6ZT12 | E3 ubiquitin-protein | 0 | 33 | 2 |
| | ligase UBR3 | | | |
| Q8IUD2 | CAST family member | 0 | 37 | 3 |
| | 1/interacting with | | | |
| | ELKS/Rab6 | | | |
| P68104 | Elongation factor-alpha 1 | 50649 | 450 | 1 |
| Q05639 | Elongation factor-alpha 2 | 50962 | 173 | 1 |
| P29692 | 1-delta elongation factor | 31281 | 88 | 3 |
| | 1 | | | |
| P26641 | Gamma elongation factor 1 | 50525 | 201 | 1 |
| P13639 | Elongation Phantom 2 | 96711 | 350 | 5 |
| O15083 | ERC protein 2 | 0 | 44 | 4 |
| P60842 | Eukaryotic initiation | 46628 | 152 | 6 |
| | factor 4A-I | | | |
| P63241 | Eukaryotic translation | 17145 | 89 | 1 |
| | initiation factor 5A-I | | | |
| Q86XX4 | FRAS1 extracellular | 454911 | 48 | 7 |
| | matrix protein | | | |
| P15311 | Ezrin | 69726 | 450 | 13 |
| P14324 | Farnesyl pyrophosphate | 48886 | 72 | 2 |
| | synthase | | | |
| Q16658 | Fascin | 55198 | 348 | 7 |
| P49327 | Fatty acid synthase | 276610 | 500 | 21 |
| A0AVI2 | Fer-1-similar to protein | 0 | 36 | 3 |
| | 5 | | | |
| Q4ZHG4 | Protein 1 containing | 206145 | 40 | 5 |
| | fibronectin type III | | | |
| | domain | | | |
| O75369 | Filamin-B | 280749 | 41 | 4 |
| Q68DA7 | Formin-1 | 159015 | 46 | 5 |
| Q5SZK8 | FRAS1-related | 0 | 33 | 5 |
| | extracellular matrix | | | |
| | protein 2 | | | |
| P04075 | Fructose bisphosphate | 39915 | 507 | 13 |
| | aldolase A | | | |
| P09972 | Fructose Bisphosphate | 39894 | 181 | 3 |
| | Aldolase C | | | |
| Q9UKJ3 | Protein 8 containing | 165218 | 39 | 4 |
| | patch domain G | | | |
| P09382 | Galectin-1 | 15080 | 43 | 2 |
| Q08380 | Galectin-3 binding | 66361 | 154 | 5 |
| | protein | | | |
| P09104 | Gamma-enolase | 47715 | 245 | 3 |
| Q92820 | Gamma-glutamyl hydrolase | 36452 | 49 | 2 |
| Q3V6T2 | Girdin | 0 | 49 | 2 |
| P14136 | Glial fibrillar acidic | 50099 | 88 | 2 |
| | protein | | | |
| P11413 | Glucose-6-phosphate 1- | 59907 | 68 | 3 |
| | dehydrogenase | | | |
| P06744 | Glucose-6-phosphate | 63579 | 660 | 2 |
| | isomerase | | | |
| Q8TCU5 | Glutamate receptor 3A | 127005 | 32 | 6 |
| | subunit [NMDA] | | | |
| P78417 | Glutathione S-transferase | 27945 | 68 | 3 |
| | omega-1 | | | |
| P04406 | Glyceraldehyde-3-phosphate | 36361 | 619 | 3 |
| | dehydrogenase | | | |
| P62826 | GTP-binding nuclear Ran | 24643 | 227 | 6 |
| | protein | | | |
| Q8NDA8 | Protein 7A containing HEAT | 0 | 34 | 3 |
| | repeat | | | |
| P0DMV8/ | 70 kDa 1A/1B protein from | 70443 | 410 | 9 |
| P0DMV9 | thermal shock | | | |
| P34931 | Protein 1 of 70 kDa thermal | 70913 | 381 | 8 |
| | shock-similar | | | |
| P34932 | Protein 4 of 70 kDa from | 95525 | 76 | 3 |
| | thermal shock | | | |
| P17066 | Protein 6 of 70 kDa from | 71608 | 320 | 5 |
| | thermal shock | | | |
| P11142 | 71 kDa cognate thermal | 71294 | 704 | 12 |
| | shock protein | | | |
| Q12931 | 75 kDa protein from thermal | 80654 | 108 | 2 |
| | shock, mitochondrial | | | |
| P04792 | Beta-1 thermal shock | 22858 | 95 | 2 |
| | protein | | | |
| P07900 | HSP 90-alpha thermal shock | 85333 | 367 | 11 |
| | protein | | | |
| P08238 | HSP 90-beta thermal shock | 0 | 518 | 3 |
| | protein | | | |
| P54652 | 70 kDa protein related | 70428 | 398 | 7 |
| | thermal shock | | | |
| Q96RW7 | Hemicentin-1 | 624433 | 47 | 6 |
| Q8NDA2 | Hemicentin-2 | 550521 | 53 | 4 |
| P09651 | Heterogeneous nuclear | 38933 | 117 | 3 |
| | ribonucleoprotein A1 | | | |
| Q32P51 | Heterogeneous nuclear- | 34471 | 117 | 3 |
| | similar A1 | | | |
| | ribonucleoprotein 2 | | | |
| P61978 | Heterogeneous nuclear | 51426 | 117 | 3 |
| | ribonucleoprotein K | | | |
| P22626 | Heterogeneous nuclear | 37576 | 122 | 4 |
| | A2/B1 ribonucleoproteins | | | |
| Q9UQL6 | Histone deacetylase 5 | 0 | 50 | 2 |
| P0C0S8 | Histone H2A type 1 | 14099 | 103 | 1 |
| P04908 | Histone H2A type 1-B/E | 14143 | 103 | 1 |
| Q93077 | Histone H2A type 1-C | 14113 | 103 | 1 |
| P20671 | Histone H2A type 1-D | 14115 | 103 | 1 |
| Q96KK5 | Histone H2A type 1-H | 13914 | 103 | 1 |
| Q99878 | Histone H2A type 1-J | 13944 | 103 | 1 |
| Q6FI13 | Histone H2A type 2-A | 14119 | 103 | 1 |
| Q16777 | Histone H2A type 2-C | 14012 | 103 | 1 |
| Q7L7L0 | Histone H2A type 3 | 14129 | 103 | 1 |
| Q9BTM1 | Histone H2A.J | 14027 | 103 | 1 |
| Q71UI9 | Histone H2A.V | 13517 | 55 | 1 |
| P0C0S5 | Histone H2A.Z | 13561 | 55 | 1 |
| Q96A08 | Histone H2B type 1-A | 14207 | 41 | 2 |
| P33778 | Histone H2B type 1-B | 13990 | 110 | 2 |
| P62807 | Histone H2B type 1- | 13946 | 148 | 2 |
| | C/E/F/G/I | | | |
| P58876 | Histone H2B type 1-D | 13976 | 148 | 2 |
| Q93079 | Histone H2B type 1-H | 13932 | 148 | 2 |
| P06899 | Histone H2B type 1-J | 13944 | 110 | 2 |
| O60814 | Histone H2B type 1-K | 13930 | 148 | 2 |
| Q99880 | Histone H2B type 1-L | 13992 | 148 | 2 |
| Q99879 | Histone H2B type 1-M | 14029 | 148 | 2 |
| Q99877 | Histone H2B type 1-N | 13962 | 148 | 2 |
| P23527 | Histone H2B type 1-O | 13946 | 110 | 2 |
| Q16778 | Histone H2B type 2-E | 13960 | 110 | 2 |
| Q5QNW6 | Histone H2B type 2-F | 13960 | 148 | 2 |
| Q8N257 | Histone H2B type 3-B | 13948 | 156 | 2 |
| P57053 | Histone H2B type F-S | 13984 | 148 | 2 |
| P68431 | Histone H3.1 | 15557 | 64 | 3 |
| Q16695 | Histone H3.1t | 15677 | 64 | 3 |
| Q71DI3 | Histone H3.2 | 15484 | 64 | 3 |
| P84243 | Histone H3.3 | 15408 | 66 | 3 |
| Q6NXT2 | Histone H3.3C | 15350 | 66 | 3 |
| P62805 | Histone H4 | 11392 | 155 | 4 |
| O14686 | Histone-lysine N- | 601158 | 69 | 6 |
| | methyltransferase MLL2 | | | |
| Q96L73 | Histone-lysine N- | 0 | 33 | 5 |
| | methyltransferase, H3 | | | |
| | lysine-36 and H4 lysine-20 | | | |
| | specific | | | |
| Q4G0P3 | Protein homolog that | 582477 | 48 | 3 |
| | induces hydrocephalus | | | |
| P00492 | Hypoxanthine-guanine | 24904 | 49 | 1 |
| | phosphoribosyltransferase | | | |
| Q14974 | Importin beta-1 subunit | 98826 | 68 | 4 |
| O00410 | Importin-5 | 125730 | 34 | 2 |
| Q14573 | Inositol 1,4,5-trisphosphate | 0 | 35 | 6 |
| | receptor type 3 | | | |
| P13645 | Keratin, type I | 59120 | 1084 | 11 |
| | cytoskeletal 10 | | | |
| Q99456 | Keratin, type I | 53832 | 101 | 3 |
| | cytoskeletal 12 | | | |
| P13646 | Keratin, type I | 50113 | 119 | 4 |
| | cytoskeletal 13 | | | |
| P02533 | Keratin, type I | 52117 | 113 | 3 |
| | cytoskeletal 14 | | | |
| P19012 | Keratin, type I | 49653 | 99 | 3 |
| | cytoskeletal 15 | | | |
| P08779 | Keratin, type I | 51754 | 261 | 3 |
| | cytoskeletal 16 | | | |
| Q04695 | Keratin, type I | 48521 | 83 | 3 |
| | cytoskeletal 17 | | | |
| P08727 | Keratin, type I | 44223 | 74 | 2 |
| | cytoskeletal 19 | | | |
| Q2M2I5 | Keratin, type I | 55751 | 58 | 3 |
| | cytoskeletal 24 | | | |
| Q7Z3Z0 | Keratin, type I | 49970 | 42 | 2 |
| | cytoskeletal 25 | | | |
| Q7Z3Y8 | Keratin, type I | 50515 | 65 | 3 |
| | cytoskeletal 27 | | | |
| Q7Z3Y7 | Keratin, type I | 51318 | 45 | 2 |
| | cytoskeletal 28 | | | |
| P35527 | Keratin, type I | 62435 | 1034 | 14 |
| | cytoskeletal 9 | | | |
| Q9NSB2 | Keratin, type II cuticular | 66102 | 71 | 2 |
| | Hb4 | | | |
| P04264 | Keratin, type II | 66301 | 1422 | 21 |
| | cytoskeletal 1 | | | |
| Q7Z794 | Keratin, type II | 62329 | 155 | 7 |
| | cytoskeletal 1b | | | |
| P35908 | Keratin, type II epidermal | 65795 | 941 | 14 |
| | cytoskeletal 2 | | | |
| Q01546 | Keratin, type II oral | 66588 | 109 | 3 |
| | cytoskeletal 2 | | | |
| P12035 | Keratin, type II | 64741 | 101 | 3 |
| | cytoskeletal 3 | | | |
| P19013 | Keratin, type II | 57816 | 177 | 3 |
| | cytoskeletal 4 | | | |
| P13647 | Keratin, type II | 62776 | 258 | 3 |
| | cytoskeletal 5 | | | |
| P02538 | Keratin, type II | 60421 | 348 | 6 |
| | cytoskeletal 6A | | | |
| P04259 | Keratin, type II | 60448 | 346 | 8 |
| | cytoskeletal 6B | | | |
| P48668 | Keratin, type II | 60401 | 341 | 8 |
| | cytoskeletal 6C | | | |
| P08729 | Keratin, type II | 51572 | 118 | 3 |
| | cytoskeletal 7 | | | |
| Q3SY84 | Keratin, type II | 0 | 89 | 3 |
| | cytoskeletal 71 | | | |
| Q14CN4 | Keratin, type II | 56640 | 72 | 3 |
| | cytoskeletal 72 | | | |
| Q86Y46 | Keratin, type II | 59611 | 151 | 5 |
| | cytoskeletal 73 | | | |
| Q7RTS7 | Keratin, type II | 0 | 83 | 3 |
| | cytoskeletal 74 | | | |
| O95678 | Keratin, type II | 59942 | 185 | 5 |
| | cytoskeletal 75 | | | |
| Q8N1N4 | Keratin, type II | 0 | 38 | 2 |
| | cytoskeletal 78 | | | |
| Q5XKE5 | Keratin, type II | 58229 | 97 | 4 |
| | cytoskeletal 79 | | | |
| P05787 | Keratin, type II | 53927 | 159 | 4 |
| | cytoskeletal 8 | | | |
| Q02241 | Kinesin-like protein KIF23 | 0 | 32 | 3 |
| Q9ULI4 | Kinesin-like protein | 0 | 47 | 5 |
| | KIF26A | | | |
| Q63ZY3 | Protein 2 containing | 0 | 42 | 2 |
| | repeat motif of KN motif | | | |
| | and anchyrine | | | |
| Q03252 | Lamin-B2 | 67985 | 33 | 2 |
| P25391 | Laminin alpha-1 subunit | 0 | 35 | 5 |
| P24043 | Laminin alpha-2 subunit | 353874 | 34 | 5 |
| P48634 | BAT2 protein rich in long | 229564 | 48 | 4 |
| | proline | | | |
| Q96JM7 | Lethal malign brain tumor | 89921 | 37 | 3 |
| | (3) - similar to protein 3 | | | |
| Q5S007 | Repeating serine rich in | 290688 | 49 | 4 |
| | leucine/threonine-protein | | | |
| | kinase 2 | | | |
| Q5VZK9 | 16A protein containing | 153228 | 36 | 3 |
| | leucine-rich repetition | | | |
| Q9UIQ6 | Leucyl-cystinyl | 0 | 31 | 2 |
| | aminopeptidase | | | |
| Q8N3X6 | Co-repressor of protein- | 0 | 37 | 2 |
| | similar ligand-dependent | | | |
| | nuclear receptor | | | |
| P00338 | L-lactate dehydrogenase A | 0 | 537 | 2 |
| | chain | | | |
| Q9BYZ2 | L-lactate dehydrogenase A- | 42508 | 147 | 2 |
| | similar to 6B | | | |
| P07195 | L-lactate dehydrogenase B | 37065 | 673 | 1 |
| | chain | | | |
| Q9NZR2 | 1B protein related to low | 536259 | 34 | 4 |
| | density lipoprotein | | | |
| | receptor | | | |
| O75096 | Protein 4 related to low | 217797 | 50 | 6 |
| | density lipoprotein | | | |
| | receptor | | | |
| Q12912 | Lymphoid restricted | 0 | 35 | 2 |
| | membrane protein | | | |
| P10619 | Lysosomal Protein | 55145 | 41 | 1 |
| P40925 | Malate dehydrogenase, | 36775 | 168 | 2 |
| | cytoplasmic | | | |
| Q8IWI9 | Gene MAX associated | 0 | 41 | 3 |
| | protein | | | |
| Q15648 | RNA polymerase II | 0 | 46 | 3 |
| | transcription subunit | | | |
| | mediator 1 | | | |
| Q96JG8 | Melanoma associated D4 | 81843 | 41 | 2 |
| | antigen | | | |
| Q13421 | Mesothelin | 69770 | 41 | 2 |
| P01033 | Metalloproteinase | 23904 | 61 | 2 |
| | inhibitor | | | |
| Q96PK2 | Factor 1 crosslinking | 675712 | 58 | 9 |
| | microtubule-actin, isoform 4 | | | |
| Q9UPN3 | Microtubule-actin | 0 | 42 | 6 |
| | crosslinking factor 1, | | | |
| | 1/2/3/5 isoforms | | | |
| P78559 | Protein 1A associated with | 0 | 35 | 4 |
| | microtubule | | | |
| P11137 | Microtubule associated | 0 | 37 | 4 |
| | protein 2 | | | |
| O60307 | Serine associated with | 144213 | 44 | 3 |
| | microtubule/threonine- | | | |
| | protein kinase 3 | | | |
| Q5JR59 | Candidate 2 tumor | 151203 | 33 | 2 |
| | suppressor associated with | | | |
| | microtubule | | | |
| Q9NU22 | Midasin | 640088 | 42 | 7 |
| Q8N4C8 | Deformation-similar to | 150873 | 45 | 2 |
| | kinase 1 | | | |
| Q99797 | Mitochondrial peptidase | 81817 | 34 | 5 |
| | intermediary | | | |
| O95819 | Protein kinase 4 activated | 0 | 37 | 2 |
| | by mitogen | | | |
| P26038 | Moesin | 68198 | 466 | 12 |
| Q8WXI7 | Mucin-16 | 2366624 | 80 | 9 |
| Q7Z5P9 | Mucin-19 | 0 | 37 | 4 |
| Q15746 | Myosin light chain kinase, | 213816 | 44 | 3 |
| | smooth muscle | | | |
| P35749 | Miosin-11 | 0 | 42 | 3 |
| Q7Z406 | Miosin-14 | 229353 | 33 | 4 |
| A7E2Y1 | Miosin-7B | 0 | 41 | 5 |
| P13535 | Miosin-8 | 224298 | 33 | 4 |
| P35579 | Miosin-9 | 228467 | 51 | 3 |
| Q9UKN7 | Miosin-XV | 398694 | 35 | 3 |
| Q92614 | Miosin-XVIIIa | 234920 | 55 | 6 |
| Q6T4R5 | Nance-Horan syndrome | 178183 | 33 | 4 |
| | protein | | | |
| Q86VF7 | Nebulin-related anchor | 198688 | 43 | 3 |
| | protein | | | |
| Q8WXH0 | Nesprin-2 | 0 | 48 | 6 |
| Q6ZNJ1 | Neurobeachin-similar to | 306093 | 44 | 3 |
| | protein 2 | | | |
| Q09666 | AHNAK protein associated with | 633316 | 77 | 7 |
| | neuroblast differentiation | | | |
| P21359 | Neurofibromin | 0 | 33 | 5 |
| Q8NEY1 | Neuron Browser 1 | 203760 | 90 | 7 |
| Q15818 | Neural Pentraxin-1 | 47727 | 119 | 4 |
| Q99574 | Neuroserpin | 46717 | 103 | 1 |
| Q86XR2 | Niban-like protein 2 | 0 | 39 | 4 |
| P29474 | Nitric oxide synthase, | 135130 | 36 | 4 |
| | endothelial | | | |
| Q15233 | Octamer binding protein | 0 | 51 | 1 |
| | containing non-POU domain | | | |
| Q8IVI9 | Nostrin | 0 | 42 | 3 |
| Q92621 | Complex pore nuclear Nup25 | 0 | 38 | 3 |
| | protein | | | |
| P15531 | Nucleoside diphosphate | 17389 | 354 | 2 |
| | kinase A | | | |
| P22392 | Nucleoside diphosphate | 17481 | 273 | 8 |
| | kinase B | | | |
| Q9NTK5 | Obg-similar to ATPase 1 | 45059 | 100 | 1 |
| Q5VST9 | Obscurin | 881261 | 56 | 5 |
| Q504Q3 | PAB-dependent poly (A) | 137244 | 37 | 3 |
| | specific ribonuclease | | | |
| | subunit 2 | | | |
| Q9BYG4 | Gamma counterpart of | 41150 | 34 | 2 |
| | defective partition 6 | | | |
| P62937 | Peptidyl-prolyl cis-trans | 18309 | 225 | 3 |
| | isomerase A | | | |
| P23284 | Peptidyl-prolyl cis-trans | 23865 | 319 | 6 |
| | isomerase B | | | |
| Q06830 | Peroxiredoxin-1 | 22372 | 332 | 4 |
| P32119 | Peroxiredoxin-2 | 22065 | 296 | 6 |
| Q13162 | Peroxiredoxin-4 | 30765 | 202 | 5 |
| P30041 | Peroxiredoxin-6 | 25197 | 379 | 13 |
| P30086 | Phosphatidylethanolamine | 21190 | 73 | 1 |
| | binding protein 1 | | | |
| P36871 | Phosphoglucomutase-1 | 61892 | 230 | 8 |
| P00558 | Phosphoglycerate kinase 1 | 45216 | 710 | 18 |
| P07205 | Phosphoglycerate kinase 2 | 45413 | 181 | 7 |
| P18669 | Phosphoglycerate mutase 1 | 28996 | 331 | 7 |
| P13797 | Plastin-3 | 71560 | 83 | 3 |
| Q15149 | Plectin | 534517 | 58 | 8 |
| P11940 | Polyadenylate Binding | 71306 | 51 | 5 |
| | Protein 1 | | | |
| Q8TDX9 | Polycystic liver disease | 0 | 44 | 4 |
| | protein-similar to 1 | | | |
| P98161 | Polycystin-1 | 468373 | 87 | 7 |
| Q6S8J3 | Member E of the Anarchine | 123582 | 307 | 12 |
| | POTE domain family | | | |
| A5A3E0 | F member of anchyrine POTE | 123692 | 35 | 3 |
| | domain family | | | |
| P0CG38 | Member I of the Anchirine | 123530 | 70 | 4 |
| | POTE family | | | |
| O60809 | Member 10 of the PRAME family | 0 | 37 | 4 |
| P02545 | Prelamin-A/C | 74540 | 81 | 3 |
| P07602 | proactivator polypeptide | 60202 | 63 | 1 |
| Q9HD20 | ATPase 13A1 carrying | 0 | 47 | 1 |
| | likely cation | | | |
| Q9Y4D8 | Likely E3 ubiquitin- | 446102 | 48 | 4 |
| | protein ligase C12orf51 | | | |
| O75592 | Likely E3 ubiquitin- | 519504 | 48 | 4 |
| | protein ligase MYCBP2 | | | |
| Q9NR48 | Likely histone-lysine N- | 0 | 33 | 4 |
| | methyltransferase ASH1L | | | |
| Q02809 | Procollagen-lysine, 2- | 84285 | 68 | 4 |
| | oxoglutarate 5-dioxygenase | | | |
| | 1 | | | |
| P07737 | Profilin-1 | 15296 | 306 | 6 |
| P12004 | Nuclear Cellular Antigen | 29252 | 97 | 2 |
| | and Proliferation | | | |
| Q9UQ80 | 2G4 protein associated | 44299 | 144 | 1 |
| | with proliferation | | | |
| Q8NBP7 | Proprotein convertase | 75866 | 423 | 2 |
| | subtilisin/kexin type 9 | | | |
| P25786 | Alpha subunit type -1 of | 29950 | 54 | 3 |
| | proteasome | | | |
| P25787 | Alpha subunit type -2 | 26076 | 140 | 4 |
| | proteasome | | | |
| P25788 | Proteasome type-3 alpha | 28787 | 43 | 3 |
| | subunit | | | |
| P25789 | Proteasome type-4 alpha | 29846 | 36 | 1 |
| | subunit | | | |
| P60900 | Alpha subunit type -6 | 27934 | 133 | 4 |
| | proteasome | | | |
| O14818 | Proteasome type-7 alpha | 28089 | 130 | 5 |
| | subunit | | | |
| P20618 | Proteasome type-1 beta | 26844 | 70 | 2 |
| | subunit | | | |
| P28070 | Proteasome type-4 beta | 29450 | 94 | 4 |
| | subunit | | | |
| P28074 | Proteasome type-5 beta | 28761 | 249 | 6 |
| | subunit | | | |
| P28072 | Proteasome type-6 beta | 25713 | 143 | 4 |
| | subunit | | | |
| Q8IVF2 | AHNAK2 protein | 620521 | 66 | 4 |
| Q9UPA5 | Bassoon protein | 0 | 52 | 6 |
| P07237 | Nucleoside diphosphate | 57567 | 177 | 7 |
| | kinase B | | | |
| P30101 | Obg-similar to ATPase 1 | 57281 | 269 | 10 |
| Q15084 | A6 disulfide isomerase | 48577 | 161 | 4 |
| | protein | | | |
| Q9Y6V0 | Piccolo protein | 0 | 36 | 6 |
| Q14160 | Protein counterpart to | 175955 | 36 | 4 |
| | write | | | |
| O95785 | Wiz protein | 180520 | 44 | 6 |
| Q9NYQ8 | Protocadherina Fat 2 | 483217 | 45 | 6 |
| Q6V0I7 | Protocadherina Fat 4 | 0 | 44 | 5 |
| Q96JQ0 | Protocadherin-16 | 0 | 34 | 4 |
| P00491 | Purine nucleoside | 32517 | 210 | 7 |
| | phosphorylase | | | |
| Q5VTE0 | Putative elongation factor | 50709 | 450 | 1 |
| | 1-alpha-similar to 3 | | | |
| Q58FF8 | HSP 90-beta 2 protein from | 44671 | 83 | 5 |
| | putative thermal shock | | | |
| P0C7M2 | Putative heterogeneous | 34469 | 117 | 3 |
| | nuclear A1-like | | | |
| | ribonucleoprotein-3 | | | |
| B8ZZ34 | Putative shisa-8 protein | 52202 | 48 | 4 |
| P46087 | Putative ribosomal RNA | 0 | 44 | 3 |
| | NOP2 methyltransferase | | | |
| Q9H853 | Putative tubulin-similar | 27915 | 77 | 1 |
| | to alpha-4B protein | | | |
| P14618 | M1/M2 isozymes of pyruvate | 58736 | 742 | 2 |
| | kinase | | | |
| P50395 | Rab GDP beta dissociation | 51287 | 318 | 7 |
| | inhibitor | | | |
| Q13671 | Ras and Rab Interactor | 0 | 37 | 3 |
| P51157 | Ras-related Rab-28 protein | 0 | 50 | 2 |
| P10586 | Tyrosine-protein | 214774 | 35 | 5 |
| | phosphatase F receptor- | | | |
| | type | | | |
| Q9P227 | Rho GTPase activation | 163666 | 38 | 2 |
| | protein 23 | | | |
| P60891 | Ribose phosphate | 35469 | 34 | 1 |
| | pyrophosphokinase 1 | | | |
| Q9H7B2 | Homolog of factor 2 of | 35907 | 47 | 3 |
| | ribosome production | | | |
| Q9P2E9 | Ribosome binding protein 1 | 153020 | 64 | 3 |
| Q8N1G1 | RNA exonuclease 1 homolog | 132891 | 53 | 3 |
| Q5TZA2 | Rootletin | 229066 | 53 | 5 |
| Q92736 | Rhinodine receptor 2 | 571865 | 36 | 7 |
| Q15413 | Ryanodine receptor 3 | 560001 | 40 | 3 |
| Q9UQ35 | Serine/arginine repetitive | 300720 | 67 | 4 |
| | matrix protein 2 | | | |
| Q9Y3S1 | Serine/threonine-protein | 244868 | 47 | 3 |
| | kinase WNK2 | | | |
| P62140 | Catalytic subunit of | 38073 | 59 | 3 |
| | serine/threonine-protein | | | |
| | phosphatase PP1-beta | | | |
| Q13315 | ATM serine protein kinase | 356828 | 37 | 5 |
| P02787 | Serotransferrin | 79494 | 35 | 2 |
| Q8IW75 | Serpin A12 | 0 | 35 | 5 |
| P35237 | Serpin B6 | 43256 | 112 | 4 |
| P50454 | Serpin H1 | 46751 | 115 | 4 |
| P02768 | Serum albumin | 71464 | 231 | 3 |
| Q9UPX8 | Protein 2 of SH3 and | 0 | 59 | 3 |
| | multiple ankyrine repeat | | | |
| | domains | | | |
| Q9BYB0 | Protein 3 of SH3 and | 0 | 57 | 4 |
| | multiple ankyrine repeat | | | |
| | domains | | | |
| Q9H2Y9 | Member 5A1 of solute | 0 | 35 | 3 |
| | family carrier of organic | | | |
| | anion carrier | | | |
| Q9H3E2 | Classifying nexin-25 | 0 | 35 | 3 |
| Q9UBP0 | Spastin | 67721 | 38 | 4 |
| Q13813 | Spectrin alpha chain, | 285976 | 51 | 4 |
| | brain | | | |
| Q9H254 | Spectrin beta chain, brain | 290693 | 86 | 4 |
| | 3 | | | |
| Q9NRC6 | Spectrin beta chain, brain | 0 | 45 | 6 |
| | 4 | | | |
| P11277 | Spectrin beta chain, | 0 | 46 | 4 |
| | erythrocyte | | | |
| Q9P0W8 | Protein 7 associated with | 68430 | 64 | 3 |
| | spermatogenesis | | | |
| Q13838 | Spliceosome RNA helicase | 49664 | 66 | 1 |
| | BAT1 | | | |
| Q12770 | Sterol regulatory element | 141819 | 48 | 2 |
| | binding protein cleavage | | | |
| | activation protein | | | |
| Q15772 | Striated muscle | 357376 | 40 | 4 |
| | preferentially expressed | | | |
| | by protein kinase | | | |
| O60279 | Protein 5 containing Sushi | 68953 | 37 | 1 |
| | domain | | | |
| Q8TER0 | Protein 1 containing | 158397 | 53 | 5 |
| | domain similar to Sushi, | | | |
| | nidogen and EGF | | | |
| Q9Y490 | Talin-1 | 272726 | 40 | 4 |
| Q9Y4G6 | Talin-2 | 274829 | 56 | 4 |
| Q5TCY1 | Tau-tubulin kinase 1 | 0 | 33 | 1 |
| P78371 | Beta subunit of protein 1 | 58040 | 35 | 1 |
| | T complex | | | |
| P50991 | Delta subunit of protein 1 | 58650 | 77 | 2 |
| | T complex | | | |
| P48643 | Epsilon subunit of protein | 60481 | 93 | 3 |
| | 1 complex T | | | |
| Q99832 | Eta subunit of complex T | 60107 | 71 | 1 |
| | protein | | | |
| P49368 | Gamma subunit of protein 1 | 61427 | 231 | 5 |
| | T complex | | | |
| P50990 | Theta subunit of protein 1 | 60450 | 70 | 5 |
| | T complex | | | |
| P40227 | Zeta subunit of protein 1 | 58676 | 141 | 5 |
| | T complex | | | |
| Q99973 | Component 1 of telomerase | 0 | 41 | 4 |
| | protein | | | |
| Q9UKZ4 | Teneurin-1 | 0 | 36 | 6 |
| Q9HBL0 | Tensin-1 | 187026 | 52 | 3 |
| Q5SRH9 | 39A tetratricpeptide | 0 | 32 | 3 |
| | repeat protein | | | |
| P10599 | Thioredoxin | 12063 | 40 | 2 |
| Q16881 | Thioredoxin reductase 1, | 71841 | 36 | 4 |
| | cytoplasmic | | | |
| P07202 | Thyroid peroxidase | 104851 | 65 | 6 |
| Q8WZ42 | Titin | 3849990 | 140 | 36 |
| P31629 | HIVEP2 transcription | 0 | 44 | 5 |
| | factor | | | |
| P02786 | Protein 1 transferrin | 85506 | 115 | 4 |
| | receptor | | | |
| Q15582 | Ig-h3 protein | 75496 | 52 | 1 |
| | transformation induced by | | | |
| | beta growth factor | | | |
| P55072 | Transitional endoplasmic | 90282 | 449 | 11 |
| | ATPase reticulum | | | |
| P29401 | Transketolase | 68739 | 742 | 1 |
| P60174 | Isomerase triosephosphate | 26986 | 441 | 3 |
| P07477 | Tripsin-1 | 27159 | 44 | 2 |
| P68363 | Alpha-1B tubulin chain | 50964 | 432 | 1 |
| Q9BQE3 | Alpha-1C tubulin chain | 50708 | 430 | 1 |
| P68366 | Alpha-4A tubulin chain | 50810 | 238 | 1 |
| Q9NY65 | Alpha-8 tubulin chain | 50906 | 138 | 1 |
| P07437 | Beta tubulin chain | 50375 | 400 | 1 |
| Q9H4B7 | Beta-1 tubulin chain | 51147 | 103 | 2 |
| P68371 | Beta-2C tubulin chain | 50551 | 54 | 1 |
| P42684 | Tyrosine protein kinase | 129675 | 54 | 5 |
| | ABL2 | | | |
| P22314 | Enzyme that activates | 119260 | 163 | 10 |
| | ubiquitin-like modifier | | | |
| Q5TEA3 | C20orf194 protein without | 134101 | 44 | 4 |
| | characterization | | | |
| Q9Y4B5 | KIAA0802 protein without | 0 | 37 | 4 |
| | characterization | | | |
| Q9Y2F5 | KIAA0947 protein without | 0 | 49 | 5 |
| | characterization | | | |
| Q2LD37 | KIAA1109 protein without | 561392 | 47 | 6 |
| | characterization | | | |
| Q5VZ46 | KIAA1614 protein without | 127754 | 35 | 3 |
| | characterization | | | |
| O75445 | Userin | 587541 | 47 | 5 |
| P46939 | Utrophin | 0 | 37 | 7 |
| Q709C8 | 13C protein associated | 0 | 62 | 4 |
| | with vacuolar protein | | | |
| | classification | | | |
| Q96QK1 | Protein 35 associated with | 92765 | 88 | 2 |
| | classification of vacuolar | | | |
| | protein | | | |
| P18206 | Vinculin | 0 | 56 | 3 |
| O43497 | Alpha-1G subunit of | 266470 | 34 | 5 |
| | voltage-dependent type T | | | |
| | calcium channel | | | |
| O75191 | Xylulose kinase | 0 | 34 | 4 |
| Q96JG9 | Zinc finger 469 protein | 414696 | 43 | 7 |
| Q9Y493 | Zonadhesin | 0 | 36 | 3 |

STM3 Interacts with HPV18 E7

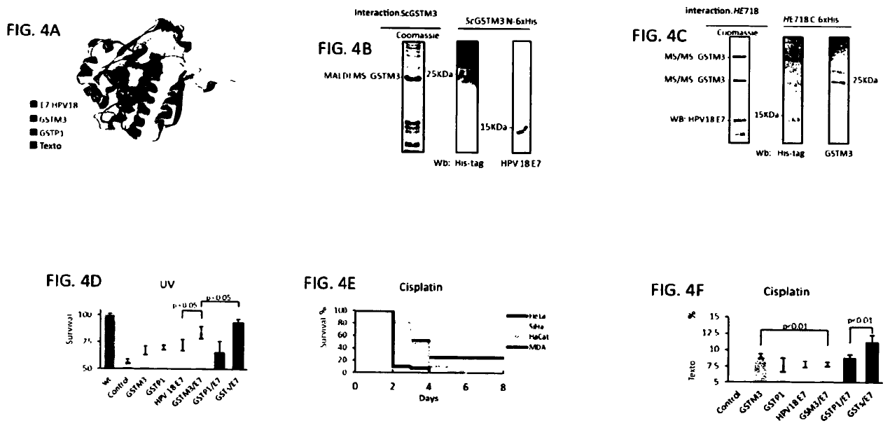

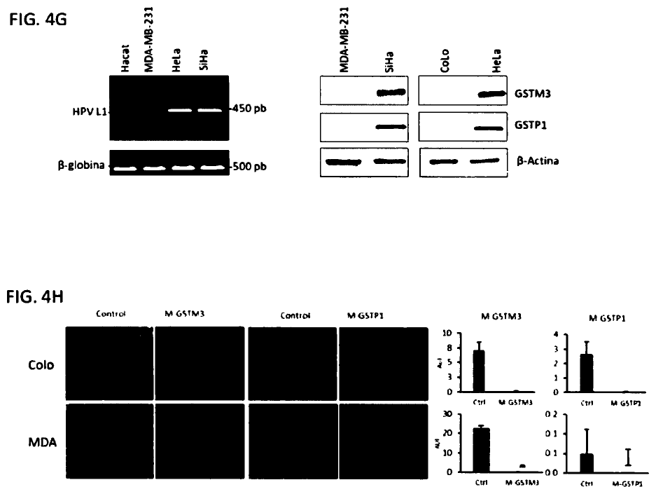

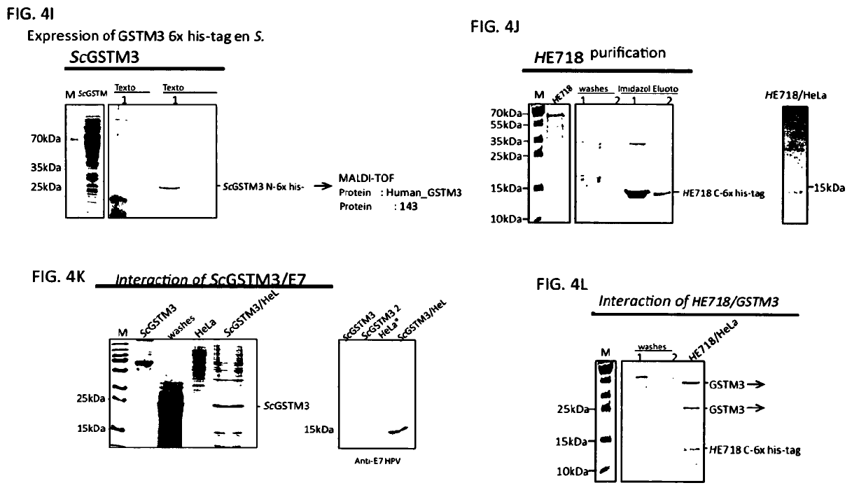

Because in Mileo, A. M., Abbruzzese, C., Mattarocci, S., Bellacchio, E., Pisano, P., Federico, A., . . . Paggi, M. G. (2009). Human Papillomavirus-16 E7 Interacts with Glutathione S-Transferase P1 and Enhances Its Role in Cell Survival. PLoS ONE, 4 (10) is shown that GSTP1 protein interacts with HPV16 E7 protein and this interaction improves cell survival, to know if GSTM3 can interact with HPV 18 E7, an alignment was performed of structural overlap between the GSTP1 and GSTM3 proteins and the E7 proteins of HPV 16 and 18 using the MAMMOTH program, followed by the Swiss PDB Viewer (Deep View) v4.1 software to visualize the results (see FIG. 4A). The alignment showed conserved and unconserved regions by comparing the distances between alpha carbons and the main amino acid chain sequences.

To demonstrate this interaction, a vector construct was generated to express a GSTM3 recombinant human protein with a histidine tag added at the N-terminal (N-6× His-tag) in S. cerevisiae (see FIG. 4I, Table 6). GSTM3 was identified through anti-His western staining and peptide mass fingerprint analysis (see FIG. 4B, FIG. 4I). After capturing the GSTM3 recombinant protein, it was incubated with a HeLa cell protein extract (HPV18-positive) (see FIG. 4J). HPV18 E7 protein co-eluted with GSTM3 N-6λ-his-tag and was identified using a specific antibody by western spotting (see FIG. 4B). To verify this interaction, an HPV18 E7 construction in S. cerevisiae was generated, but it was possible to obtain a stable strain that expressed the protein. A plasmid construct was then generated that expressed an HPV18 E7 C-6λ-his-tag recombinant protein in the HeLa cell line and performed a protein interaction (“pull-down”) assay (see FIG. 4K). The results showed that GSTM3 can interact with HPV18 E7 (see FIG. 4C, FIG. 4L) and that this interaction acts similarly to the interaction between GSTP1 and E7 proteins of HPV16 (see FIG. 4A).

TABLE 6
List of primers used to generate
recombinant GSTM3 protein.

| Name | Sequence 5′ > 3′ |
|---|---|
| M3-1 | ATGTCGTGCGAGTCGTCTATGGTTCTCGGGTACTGGGATATTC |
| | GTGGGCTGGCGCACGCCATCCGCCTGCTCCTGG |
| M3-2 | CATAGTCAGGAGCTTCCCCGCACGTGTACCGTTTCTCCTCATA |
| | AGAGGTATCCGTGAACTCCAGGAGCAGGCGGATGG |
| M3-3 | GGAAGCTCCTGACTATGATCGAAGCCAATGGCTGGATGTGAAA |
| | TTCAAGCTAGACCTGGACTTTCCTAATCTGCCCTACC |
| M3-4 | GCTTGCGAGCGATGTAGCGCAAGATGGCATTGCTCTGGGTGAT |
| | CTTGTTCTTCCCATCCAGGAGGTAGGGCAGATTAGG |
| M3-5 | GCTACATCGCTCGCAAGCACAACATGTGTGGTGAGACTGAAGA |
| | AGAAAAGATTCGAGTGGACATCATAGAGAACC |
| M3-6 | CAGTTTTTCGTGGTCAGAGCTGTAACAGAGCCTTATCAGTTGT |
| | GTGCGGAAATCCATTACTTGGTTCTCTATGATGTCC |
| M3-7 | GCTCTGACCACGAAAAACTGAAGCCTCAGTACTTGGAAGAGCT |
| | ACCTGGACAACTGAAACAATTCTCCATGTTTCTGG |
| M3-8 | GGTGAGAAAATCCACAAAGGTGAGCTTTTCCCCGGCAAACCAT |
| | GAGAATTTCCCCAGAAACATGGAGAATTG |
| M3-9 | CCTTTGTGGATTTTCTCACCTATGATATCTTGGATCAGAACCG |
| | TATATTTGACCCCAAGTGCCTGGATGAGTTCC |
| M3-10 | CCAAGTGCCTGGATGAGTTCCCAAACCTGAAGGCTTTCATGTG |
| | CCGTTTTGAGGCTTTGGAGAAAATCGCTGCC |
| M3-11 | CCACTGGGCCATCTTGTTGTTGATGGGCATCTTGCAGAACTGA |
| | TCAGACTGTAAGTAGGCAGCGATTTTCTCCAAAGC |
| M3-12 | CCATCAACAACAAGATGGCCCAGTGGGGCAACAAGCCTATATG |
| | CTGA |
| GSTM3- | ATAGAC AAGCTT AACAAAATGTCTGGGTCGTCG |
| HindIII | CACCATCACCACCATCAT TCGTGCGAGTCGTCTATGG |
| GSTM3- | ATACAA GGATCC TCAGCATATAGGCTTGTTGC |
| BamHI | |

GSTM3-HindIII. This oligonucleotide contains the HindIII restriction site (in bold), yeast consensus sequence at the translational start site and codons for 6 histidines (bold and underlined).

GSTM3-BamHI. This oligonucleotide contains the BamHI restriction site (bold).

Primers used for amplification and cloning of the HPV18 E7 gene

E718-1
ATG CAT GGA CCT AAG GCA ACC ATT
E718-2*
CTG CTG GGA TGC ACA CCA
E7-18-Hind III
ATA CAA AAG CTT ATG CAT GGA CCT AA
E7HPV18-his*
GAT GGT GAT GAT G CT GCT GG
Univ His-Tag BamH I*
TAC GTG GAT CCT AGT GGT GAT GGT G

E7-18-Hind III. This oligo contains the Hind III restriction site (bold) and initial HPV 18 sequence (bold).

E7HPV18-his. This oligo contains a fragment of 6× histidine sequence (bold).

Univ His-Tag BamHI. This oligo contains the BamHI restriction site (bold) and 6× histidine sequence fragment (bold and underlined).

Once the interaction of the GSTM3 protein with the HPV18 E7 protein was demonstrated, the relevance of this interaction in cell survival was evaluated. For this purpose, a stress sensitivity test with UV was developed in a breast cancer cell line MDA-MB-231 which is negative for HPV, and the expression of GSTM3 and GSTP1 proteins (see FIG. 4G). Using recombinant GST and E7 HPV18 proteins, a phenotype analysis was performed by exogenous protein complementation (PAEP). This analysis demonstrated that under stress of UV radiation (15 seconds UV, IC50), the GSTM3/HPV18 E7 cells exhibited a survival rate of 84.1%, while the GSTP1/GSTM3/E7 cells exhibited a survival rate of 93.7% after a 24-hour recovery period. These results indicate that there is a synergistic effect between GST and viral proteins (see FIG. 4D). An in vitro assay was performed wherein CC cell lines and negative cell lines were exposed to 6 mM cisplatin. This concentration of cisplatin completely killed the MDA-MB-231 cell line on the 4th day of treatment. For the HaCaT cell line, the total number of dead cells was on the 6th day (see FIG. 4E). Surprisingly, the cell lines that coexpress GST and HPV E7 survived for at least eight days after treatment (SiHa 17% and HeLa 24% confluence) (see FIG. 4E). To demonstrate that the GST/HPV18 E7 interaction was responsible for this resistance, a PAEP assay was performed using MDA-MB-231 cells that included recombinant proteins GSTM3, GSTP1 and E7 of HPV18. The results confirmed that cell lines expressing members of the HPV GST and E7 family of proteins have an advantage in terms of cell survival when treated with a xenobiotic agent (see FIGS. 4E, 4F). An increase in the survival of cells expressing any of these proteins (HPV18 E7, GSTM3 or GSTP1) was observed; however, the greatest increase in survival was observed when both GST and HPV18 E7 were present (see FIG. 4F).}

“Inhibition of Genes” (Knock-Down) GST In Vitro and In Vivo Using Antisense Oligonucleotides

As an example, without being limiting, the antisense oligonucleotides of vivo-morpholinos were designed to inhibit the therapeutic targets of the GSTs starting from the 5′UTR region of the messenger RNAs of the GSTM3 and GSTP1 and the ATG start codon and include 25 nucleotides, but this region is not limiting being able to use a range of 15-50 nucleotides, preferably from 18 to 30, more preferably from to 25, and bases 1-773, preferably close to the start codon, of the GSTP1 gene and for GSTM3 from base 1-4144, preferably close to the start codon, with a similarity of 100-50% of both sequences.

It is obvious that a skilled person in the art can employ any chemical modification of the RNA or DNA sequences to inhibit GSTs, for example: 2′MOE, 2′MO, PNA, LNA, Phosphorothioate, 2′-F, etc. They are commercially available. The preferred GSTs in the present invention, without being limiting thereof, are GSTM3 and GSTP1 with the sequence of antisense oligonucleotide (OAS) 5′-TAGACGACTCGCACGACATGGTGAC-3′ (56% CG−) and 5′-AATAGACCACGGTGTAGGGCG-3G′ (56% CG), respectively.

For both in vitro and in vivo tests both antisense oligonucleotides were dissolved in sterile PBS saline phosphate buffer at pH 7.5.

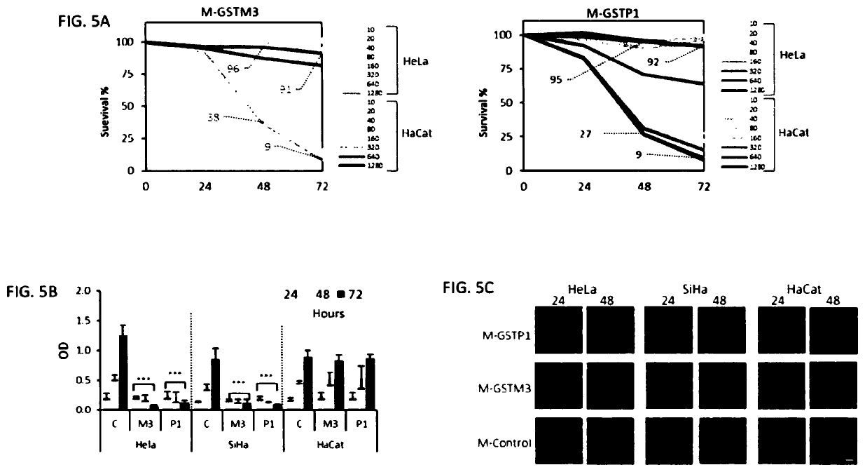

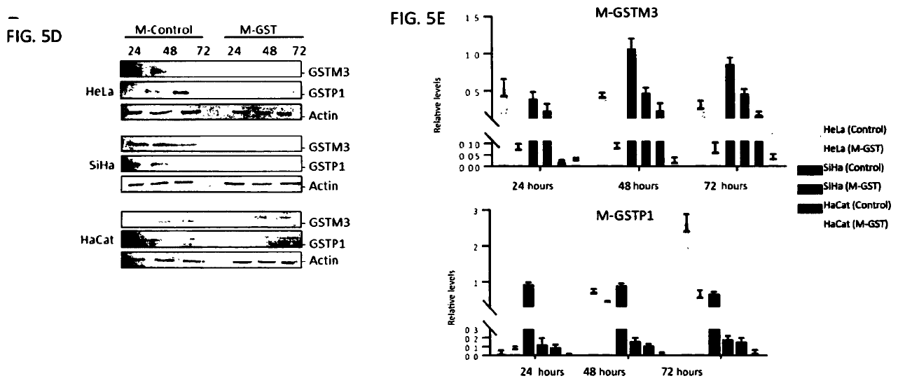

In order to evaluate the effect of GSTM3 and GSTP1 on CC cell lines, the expression of both proteins was inhibited by means of antisense oligonucleotides (OAS) and a random sequence was used as a control. To evaluate the inhibition of GSTs, eight doses were evaluated for each antisense oligonucleotide (OAS) in culture with two cell lines, HeLa and HaCaT (negative control). The concentrations used were between the range of 10 to 1,280 ng/mL and were incorporated into the culture medium. Subsequently, cell proliferation was evaluated at three different times at 24, 48 and 72 hours. It was observed that HaCaT cells were not affected by treatment with any antisense oligonucleotide during the analysis period. However, a slight loss of survival was noted with the highest dose (1,280 ng/mL). In HeLa cells, viability losses were observed after 48 hours of treatment in all doses of OAS-GSTM3 (see FIG. 5A). After 72 hours, the highest treatment doses (640 and 1,280 ng/mL) showed a survival of less than 10% compared to the control cells. Similar results were obtained for treatment with OAS-GSTP1 in both cells.

To evaluate the cellular response in other cell lines, the dose of 640 ng/mL was selected, because this is the highest dose that did not affect the HaCaT cell line. In addition, treatment was performed with 640 ng/mL in the SiHa CC cell line (see FIG. 5B). A very similar response was obtained between cancer cell lines, indicating that both GST proteins are essential for cell survival in CC, but not for HaCaT (non-cancerous) cells. To validate the effectiveness of the elimination treatment, a western blot analysis was performed on the three cell lines for both proteins (see FIGS. 5D-5E). Immunoblotting revealed that both proteins were in fact negatively regulated during all times of treatment in all three cell lines. In addition, the cell viability in the three cell lines was evaluated after 24 and 48 hours of treatment at the dose of 640 ng/mL of the two antisense oligonucleotides (see FIG. 5C). A live/dead cell assay was carried out based on the staining of Syto9/propidium iodide. The results confirmed that HaCaT cells were not affected by the treatment. Both cancer cells were similarly affected. Together, these results demonstrate that HaCaT cells have an alternative mechanism of cell maintenance that is compromised to CC cells.

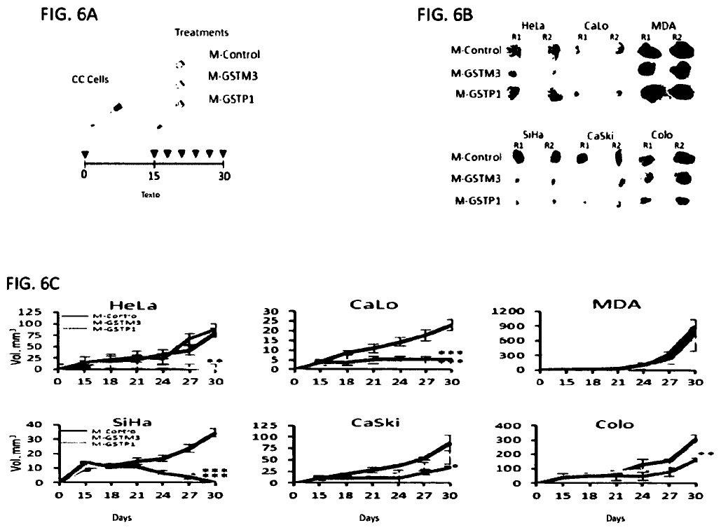

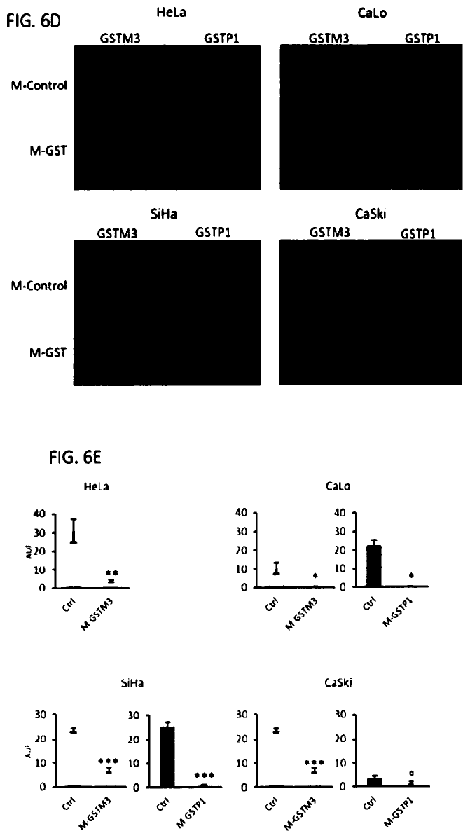

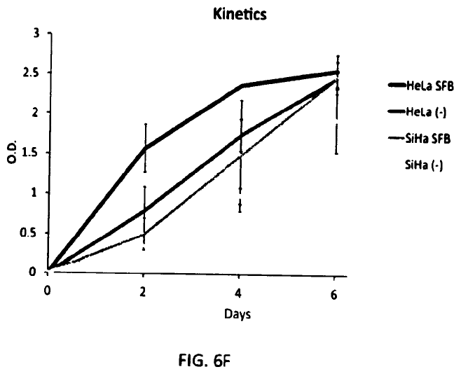

For in vivo assays, 15 days after tumor inoculation in mice, six doses of 400 ng/500 μL were injected intratumorally, every third day. On day 30, tumors were collected for later analysis. A randomized antisense oligonucleotide was used as a control in both in vitro and in vivo assays. The sequences of the antisense oligonucleotides used in FIGS. 6A-6E.

Loss of GST Inhibits Tumor Progression in Cervical Cancer

To demonstrate the importance of GST during tumor progression (PT), the effects of treatments with antisense oligonucleotides in a murine model were examined (see FIG. 6A). For this, the antisense oligonucleotides (OAS-GSTM3, OAS-GSTP1, and OAS-Control) were used and four CC cell lines were treated (two HPV16-positive lines, SiHa and CaSki, and two HPV18-positive lines, HeLa and Calo), as well as two different cell lines to CC, one of breast cancer (MDA-MB-231) and one of colon (COLO 205). The results of the in vivo and in vitro analyzes were correlated with each other, showing a drastic decrease in the volume in the tumor cell lines of CC (see FIGS. 6B-6C). However, the results for HeLa tumors were different from those performed in vitro. HeLa tumors only expressed GSTM3, but not GSTP1 (see FIGS. 5D-5E). Therefore, treatment with OAS-GSTP1 in HeLa tumors did not affect PT, which confirmed that GSTP1 is not expressed in these tumors. On the other hand, treatment with OAS-GSTM3 in HeLa tumors dramatically decreased tumor volume. Compared to the treatment with the control antisense oligonucleotide, the tumor volume of HeLa with OAS-GSTM3 was 14 times lower (see FIGS. 6B-6E).

In CaLo tumors, both proteins expressed as GSTM3 and GSTP1 were found (see FIGS. 6D-6E). Treatment of these tumors with the antisense oligonucleotides against GSTM3 and GSTP1 resulted in a decrease in tumor volume 10 and 6 times, respectively (see FIGS. 6B-6C). In the case of SiHa tumors, which express both proteins (see FIGS. 6D-6E), it was observed that the greatest decreases in tumor volume after treatment with both antisense oligonucleotides were 43 and 62 fold decreases for OAS-GSTM3 and OAS-GSTP1, respectively (see FIGS. 6B-6C). CaSki cell line control tumors also expressed both proteins. In the treated tumors, it was observed that the levels of GSTM3 and GSTP1 did not decrease as much as in other tumors that expressed these proteins (see FIGS. 6D-6E). Treatment with OAS-GSTP-1 resulted in a tumor volume reduction of 2.6 times compared to the control. In the case of treatment with OAS-GSTM3, no significant differences were observed between the volume of control tumors and tumors treated with OAS-GSTM3 (see FIGS. 6B-6C). The low efficacy in the inhibition of protein expression by treatment, particularly for GSTM3, was responsible for the poor response in tumor reduction, so the remaining GSTM3 is sufficient to provide a protective effect to tumor cells.

In addition, the response to the treatment of tumors of two cell lines of different origins, MDA-MB-231 of breast cancer and COLO of colon cancer, was also studied. Both tumors exhibited low GSTP1 expression compared to CC tumors (see FIGS. 6D-6E). However, the treated COLO tumors had levels 1.9 times lower than the control (see FIGS. 6B-6C). In the case of MDA-MB-231, GSTP1 levels were barely detectable in control tumors and, as a consequence, treatment with OAS-GSTP1 did not affect PT (see FIGS. 6B-6C). Tumors of both cell lines (COLO and MDA) expressed GSTM3 and in both cases, treatment with the antisense oligonucleotide significantly reduced protein levels.

GSTM3 and GSTP1 Regulate the MAP Kinase Proteins pJNK and pp38.

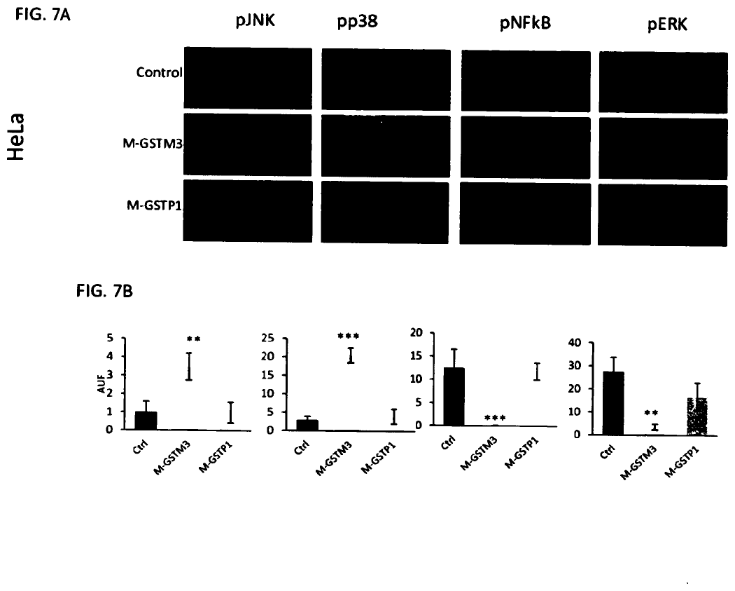

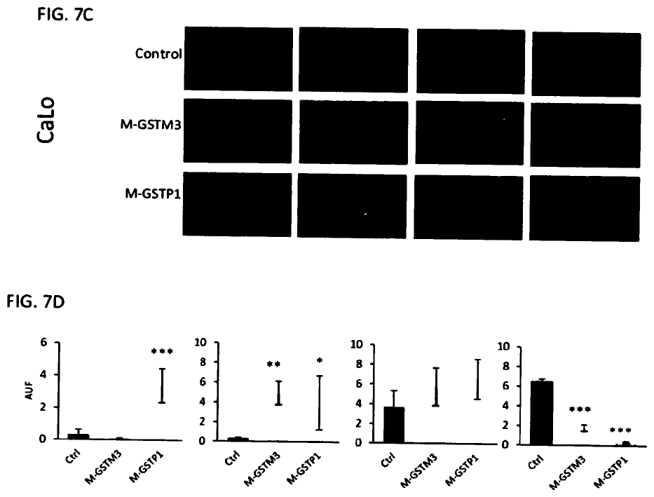

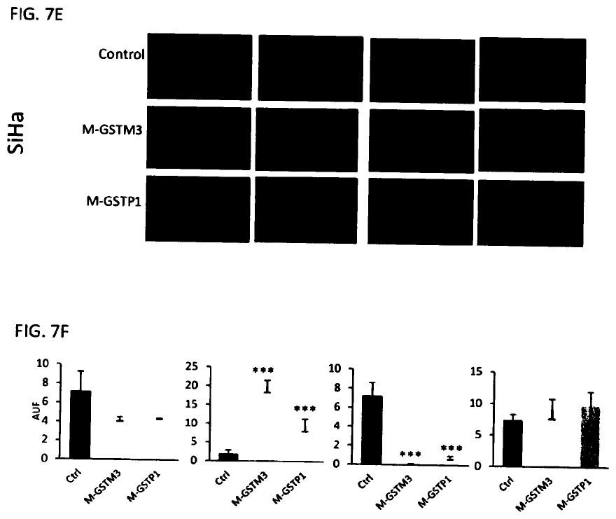

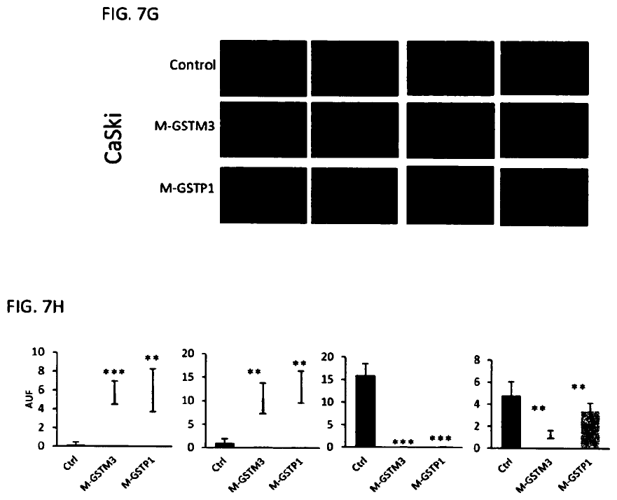

The effects of the deactivation of GSTM3 and GSTP1 on the activation of pJNK and pp38 and the phosphorylation of p65 and pERK (from the NF-κB pathway) during CC PT were analyzed (see FIGS. 7A-7H). Protein expression was analyzed through immunohistochemical assays in all CC tumors treated with antisense oligonucleotides (OAS-GSTM3, OAS-GSTP1 and OAS-control). OAS-GST treatments resulted in phosphorylation and activation of pJNK and pp38 MAP kinases. HeLa tumors that only express GSTM3 and, therefore, only responded to treatment with the OAS-GSTM3 antisense oligonucleotide, showing increased phosphorylation of JNK and p38 (see FIGS. 7A-7B). On the other hand, the CaLo and SiHa tumors only showed p38 phosphorylation, with the two treatments OAS-GSTM3 and OAS-GSTP1; CaLo (see FIGS. 7C-7D); and SiHa (see FIGS. 7E-7F). For CaSki tumors, both MAPK were positively regulated after treatment with OAS-GST (see FIGS. 7G-7H).

GSTM3 and GSTP1 Regulate Cell Survival by Inactivating NF-κB and pERK

The inactivation of the ERK protein and p65 NF-κB was examined (see FIGS. 7A-7H). HeLa tumors treated with OAS-GSTM3 showed inactivation of both proteins (see FIGS. 7A-7B).

In CaLo tumors, only pERK was inactivated after treatment with any of the antisense oligonucleotides for GST (see FIGS. 7C-7D). For SiHa tumors, only NF-κB was inactivated by any of the treatments (see FIGS. 7E-7F). In CaSki tumors, both proteins were inactivated after any treatment (see FIGS. 7G-7H). Inhibition of GSTM3 and GSTP1 proteins induced apoptosis and decreased cell survival through the NFκB and MAP kinase pathways.

GST Expression Analysis in Tissue Samples from CC Patients

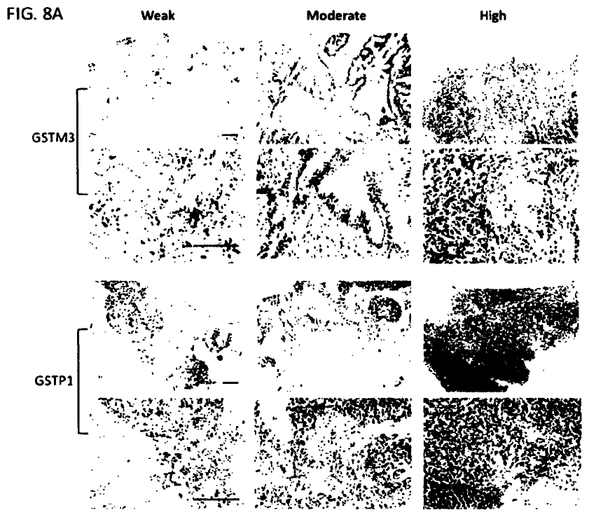

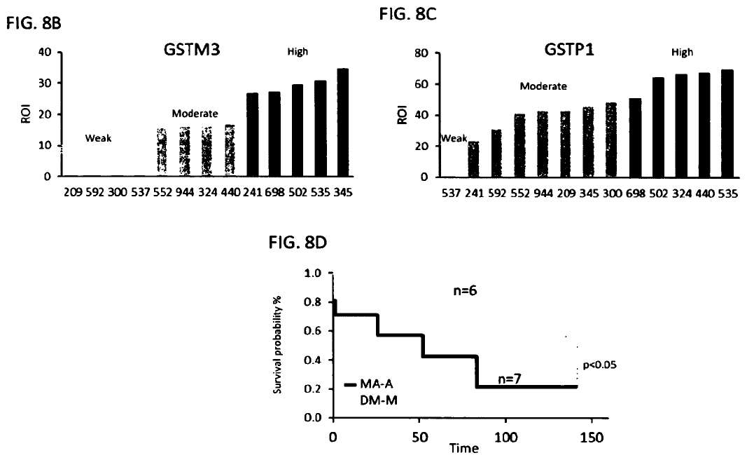

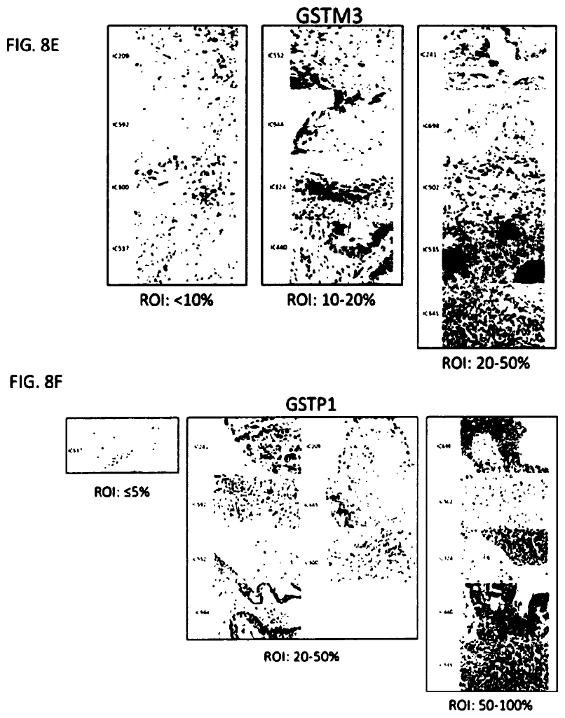

A follow-up study of 13 patients with CC who had undergone chemotherapy was performed (see FIGS. 8E-8F). Protein expression analyzes were performed for GSTM3 and GSTP1 using immunohistochemistry (IHC). In this study, the percentage of the region of interest (ROI) that was immunopositive was analyzed. Surprisingly, all patients expressed both proteins, but with great variability with respect to the percentage of ROI (see FIG. 8A, FIGS. 8E-8F). Patients were arbitrarily categorized into three groups based on the percentage of ROI: weak, moderate and high for GSTM3 and GSTP1 (see FIGS. 8B-8C). Next, an association analysis of GST expression and patient survival was performed and two groups were generated: weak-moderate for GSTM3 and moderate for GSTP1 (DM-M), and another group with moderate-high values for GSTM3 and high values for GSTP1 (MA-A) (see FIG. 8D). The results showed that the expression of GSTM3 and GSTP1 could significantly influence the survival of patients with CC. A clear correlation is observed between patient survival and GST protein expression. Patients who showed a weak to moderate expression (DM-M) showed a significantly higher survival rate than patients who exhibited a moderate to high GST expression (MA-A) (see FIG. 8D). The results are shown in Table 7 below.

TABLE 7
ROI of GST proteins of patients with CC

| | | | Mean sum | Total | | | | | Mean sum | Total | | |
|---|---|---|---|---|---|---|---|---|---|---|---|---|
| | | Mean sum | without | Area | Mean | | | Mean sum | without | Area | Mean | |
| | | of stain | stain | Cell | area | | | of stain | stain | Cell | area | |
| | | (Area) | (Area) | Area | fraction | Classifi- | | (Area) | (Area) | Area | fraction | Classifi- |
| Protein | ID | (μm²) | (μm²) | (μm²) | ROI % | cation | Protein | (μm²) | (μm²) | (μm²) | ROI % | cation |
| GSTM3 | 502 | 311,243.79 | 735,599.42 | 1,046,843.21 | 29.73 | High | GSTP1 | 814,269.62 | 442,021.41 | 1,256,291.03 | 64.8 | High |
| | 300 | 36,571.91 | 527,141.79 | 563,713.70 | 6.49 | Weak | | 536,105.53 | 566,210.48 | 1,102,316.01 | 48.6 | Moder- |
| | | | | | | | | | | | | ate |
| | 698 | 67,007.39 | 177,911.74 | 244,919.13 | 27.36 | High | | 511,678.95 | 488,372.19 | 1,000,051.14 | 51.2 | High |
| | 324 | 113,134.55 | 580,128.64 | 693,263.20 | 16.32 | Moder- | | 895,090.00 | 448,948.52 | 1,344,038.52 | 66.6 | High |
| | | | | | | ate | | | | | | |
| | 345 | 450,109.91 | 838,099.43 | 1,288,209.34 | 34.94 | High | | 577,972.52 | 686,381.85 | 1,264,354.38 | 45.7 | Moder- |
| | | | | | | | | | | | | ate |
| | 944 | 126,711.35 | 655,983.41 | 782,694.77 | 16.19 | Moder- | | 516,617.31 | 696,180.99 | 1,212,798.30 | 42.6 | Moder- |
| | | | | | | ate | | | | | | ate |
| | 535 | 253,528.44 | 564,860.42 | 818,388.86 | 30.98 | High | | 869,484.57 | 380,402.05 | 1,249,886.62 | 69.6 | High |
| | 241 | 236,100.04 | 639,148.15 | 875,248.19 | 26.98 | High | | 232,216.69 | 761,975.89 | 994,192.58 | 23.4 | Moder- |
| | | | | | | | | | | | | ate |
| | 209 | 22,949.68 | 484,613.05 | 507,562.73 | 4.52 | Weak | | 265,209.56 | 355,823.84 | 621,033.40 | 42.7 | Moder- |
| | | | | | | | | | | | | ate |
| | 592 | 39,824.78 | 830,366.61 | 870,191.39 | 4.58 | Weak | | 221,299.66 | 490,337.19 | 711,636.84 | 31.1 | Moder- |
| | | | | | | | | | | | | ate |
| | 440 | 200,506.09 | 985,348.59 | 1,185,854.68 | 16.91 | Moder- | | 877,113.35 | 420,341.69 | 1,297,455.04 | 67.6 | High |
| | | | | | | ate | | | | | | |
| | 552 | 113,462.72 | 617,323.87 | 730,786.59 | 15.53 | Moder- | | 240,094.59 | 345,836.90 | 585,931.49 | 41.0 | Moder- |
| | | | | | | ate | | | | | | ate |
| | 537 | 72,269.62 | 680,970.07 | 753,239.69 | 9.59 | Weak | | 37,216.89 | 708,046.98 | 745,263.87 | 5.0 | Weak |

The results obtained indicate that patients with cervical cancer expressed the GSTM3 and GSTP1 proteins, and that the expression of the tissue samples is related to survival. Therefore, it is concluded that the increase of these proteins is involved with the prognosis of the patient being unfavorable is overexpression.

The present invention provides evidence for the identification and inhibition of the expression of GSTM3 and GSTP1. In the results of the cultures, the cervical cancer cells were drastically affected by the blockade of both GST, while the HaCaT (non-cancerous) control cells were not affected by the inhibition of these proteins, whereby the GSTM3 and GSTP1 are crucial for the survival and proliferation of cancer cells in culture. In inoculated tumors, it was observed that in those CC cell lines expressing at least one of these proteins, the tumor volume decreased dramatically after treatment with antisense oligonucleotides.

The present invention demonstrates that inhibition of GSTM3 or GSTP1 activates the signaling of JNK and p38, which leads the cells to apoptosis and, therefore, decreases the tumor volume. On the other hand, it was observed that the inactivation of NF-κB and/or ERK after GST inhibition inhibits cell survival.

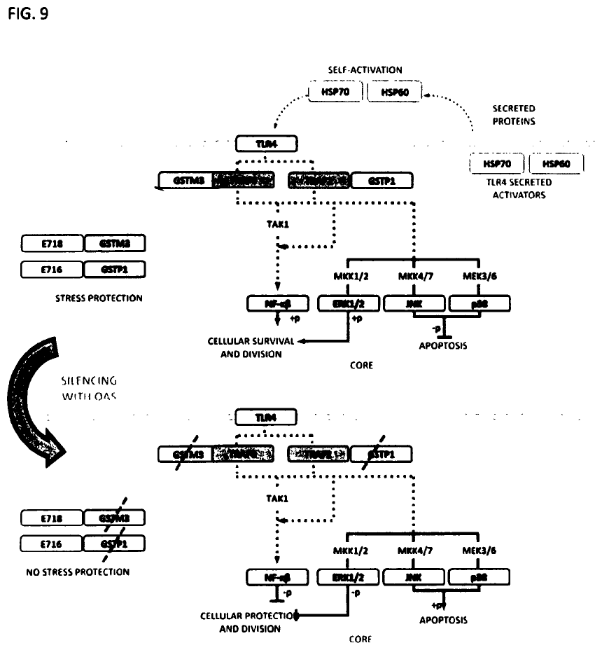

The present invention shows that there is a strong association of the overexpression of GSTM3 and GSTP1 proteins and the survival of patients. The results obtained in vitro and in vivo are consistent with the clinical data, since the survival of patients with CC was associated with high levels of GST protein (see FIG. 7C). These data also agreed with studies on bladder and colon cancer in which it was found that GSTM3 protein overexpression was associated with a reduced patient survival rate. Therefore, the present invention presents a mechanism by which, CC cells use GST proteins to prevent apoptosis and activate cell survival and proliferation. In addition, this response is affected by the inhibition of these proteins (see FIG. 9).

### EXAMPLES

The following examples will allow us to understand the present invention even more, as well as to show the best method of carrying it out. It should be understood that said examples are illustrative of the present invention and not in any way limiting the scope thereof. References cited herein should be construed as being expressly incorporated therein.

It should also be understood that the methods and techniques of protein extraction, and immunodetection, as well as the protocols for sample preparation, preparative two-dimensional gel electrophoresis, image analysis and protein identification through MALDI mass spectrometry, which are not specifically described in the examples, are reported in the aforementioned literature and are known to those skilled in the art. For example, phenolic protein extraction is described in Hurkman, W. J., & Tanaka, C. K. (1986).

Solubilization of plant membrane proteins for analysis by two-dimensional gel electrophoresis. Plant physiology, 81(3), 802-806; Encarnación, S., Guzmán, Y., Dunn, M. F., Hernández, M., Vargas, M. del C., & Mora, J. (2003). Proteome analysis of aerobic and fermentative metabolism in Rhizobium etli CE3. In Proteomics (Vol 3, bll 1077-1085), Klose, J., & Kobalz, U. (1995). Two-dimensional electrophoresis of proteins: an updated protocol and implications for a functional analysis of the genome. Electrophoresis, 16(6), 1034-1059.

Tumor Generation

Female athymic nude mice 4-6 weeks old (BALB/c Nu/Nu) were used and subcutaneously injected with 10′ tumor cells in 500 μL of RPMI 1640 medium without FBS and collected in 30, 45 and 50 days. The tumors were measured using a Vernier caliper, and the tumor volume was obtained by calculating the volume of an ellipsoid as π/6 (L*W*H), wherein L: length, W: width and H.

Tumor Protein Extraction, Proteomic Analysis and Mass Spectrometry.

Tumor tissue samples were extracted by macerating them in liquid nitrogen and a cocktail of protease inhibitor and phosphatases, followed by sonication on ice, to then carry out the extraction of phenolic proteins by extraction with RIPA buffer. Subsequently, the protein expression analysis is performed using anti-GSTM3 and anti-GSTP1 antibodies and then visualized by immunodetection techniques such as: western staining (western blot), immunohistochemistry, ELISA, etc.

Extracellular Protein Extraction In Vitro and Ex Vivo.

Cell lines were cultured with RPMI 1640 advanced serum free up to 70-80% confluence. The medium was removed and rinsed three times with sterile saline: 0.9% NaCl (w/v). After washing, FBS-free RPMI 1640 medium without fresh red phenol (Gibco) was added and incubated for 20 h. Later, the medium was recovered and centrifuged at 2,000 g for 5 minutes. The supernatant was passed through a 0.22 μm pore size PVDF membrane (Millex, Millipore) and stored at −70° C. until later use. For extracellular proteins from xenograft tumors, HeLa and SiHa tumors were inoculated with 10⁷ cells. After 30, 45 and 50 days after inoculation, the tumors were collected and washed 3 times with saline. The procedure followed to extract secreted proteins from tumors was performed as previously described for the cells in culture and the supernatant was stored at −70° C. until later use.

Identification of Secreted Proteins Through LC-MS/NS

The identification of secreted proteins in cell lines was separated in SDS-PAGE. The generated peptides were analyzed in a nanoLC-MS/MS system (Q-TOF Synapt G2 MS; Waters), the identification of the peptides and proteins was done through the MASCOT Distiller interface (Matrix Science), and the database Swiss-Prot and NCBI.

Signal Peptide Analysis

For the analysis of the signal peptide a bioinformatic program called SignalP 4.1 was used, which predicts the presence and location of signal peptide sites in amino acid sequences. The method predicts and identifies the export sites of the signal peptide based on physicochemical features and a combination of neural networks (NN) and hidden Markov models (HMM).

Coimmunoprecipitation and Immunostaining (Immunoblot)

The HeLa tumor was collected at 50 days and stored at 80° C. until use. After the tumor sample, they were macerated in liquid nitrogen and lysed with 500 μL RIPA buffer (10 mM Tris, 1 mM EDTA, 1% NP40, 0.1% sodium deoxycholate, 140 mM NaCl) and supplemented with inhibitors of protease and phosphatase (10 mM β-glycerophosphate, 10 mM Na₃VO₄, 10 mM sodium fluoride). Total cell lysates were centrifuged at 13,000 g for 5 min to settle the insoluble material. The lysates are incubated 2 hours with sepharose protein A, normalized for the total protein concentration (10 μg protein) using SDS-PAGE. Protein candidate antibodies (GSTM3 and TRAF6) were immunoprecipitated by incubating lysates with 6 μL antibody conjugated sepharose overnight at 4° C. The beads were washed 3 times with 500 μL lysis buffer. Co-immunoprecipitant proteins were resolved in 12% SDS-PAGE. GSTM3 and TRAF6 levels were detected by immunoblotting using previously described anti-antibodies.

Commercial antibodies to analyze Western staining (western blot) proteins were selected from anti-GSTM3 (Abcam, ab67530, 1:10,000), anti-GSTP1 (Abcam, ab53943, 1:10,000), anti-TLR4 (Biolegen, 312804, 1:10,000), anti-TRAF6 (Abcam, ab13853, 1:10,000), anti-NF-Kb P65 (SC-378, 1:1,000), anti IKB-α (SC-371, 1:1,000), anti-JNK (sc-1648, 1:1,000), antiERK (sc-94, 1:1,000), anti p38 (sc-535, 1:1,000), anti-NF-κB phospho p65 (sc-101752, 1:1,000), anti-phospho-JNK (sc-6254, 1:1,000), anti-phospho-ERK (sc-7383, 1:1,000), phospho-p38 (sc-7973, 1:1,000), anti-phospho-IKB-α (Cell signaling, 1:1,000), anti-HSP70 and HSP60 (Biolegen, 648005 and 681502, 1:10,000), HPV18 E7 (Abcam, ab38743, 1:1,000), anti-His tag antibody (Invitrogen, 372900, 1:5,000). The cells are lysed in a buffer containing 100 mM Tris (pH 8.6); 4% SDS; 100 mM DTT; a protease inhibitor cocktail. To ensure lysis, pulses are given with a sonicator for 1 second for DNA fragmentation. The proteins are electrophoresed in SDS-PAGE from 12% to 15% and transferred to nitrocellulose membranes using a semi-dry system. The already transferred membranes are blocked with 5% skim milk or bovine serum albumin in a Tris saline buffer containing Tween 20 (TBST) for 15 minutes at 4° C., washed three times in TBST. Subsequently, albumin or skim milk is removed and washed 3 times with TBST, then the membrane is incubated with the primary antibody (anti-GSTM3 or anti-GSTP1) at 4° C. overnight and tested with primary antibody diluted and incubated at 4° C. overnight. After removing the primary antibody the membranes were incubated with a secondary antibody conjugated to peroxidase for 2 h and then the membrane was revealed with a solution of Carbazol (27.2% Carbazol Stock, 72.6% acetate buffer, 0.2% H2O2), Carbazol Stock: N,N-Dimethylformamide≥98% and 3-Amino-9-ethylcarbazole (Sigma-Aldrich) in a 1:8 (w/v) ratio to generate a red/brown. Relative quantifications were performed with ImageJ software.

To analyze tissue samples by immunohistochemistry (IHC), the percentage of the region of interest (ROI) that is the region that gives positive expression of GSTs is analyzed. The medical records were reviewed, taking into account the patient's previous medical history. All cases were subjected to an immunohistochemical analysis using anti-GSTM3 (Abcam, ab67530, 1:1,000) and, anti-GSTP1 (Abcam, ab53943, 1:1,000). The paraffin blocks were sampled at a tissue thickness of 5 μm and produced in duplicate for each slide. The analysis was performed in an automated immunocontainer (Ventana Medical Systems). Three parts of the tumor were evaluated separately in each sample, as was the presence of staining in the tumor cells. In this study, we analyzed the percentage of the region of interest (ROI) stained by the antibodies, as estimated using the CellSens (Olympus) software. The samples were divided into two groups to assess the association of protein expression and patient survival: (W-M) consisting of a weak ROI for GSTM3 and moderate ROI for GSTP1; and (MH-H) which groups a moderate/high ROI for GSTM3 and high ROI for GSTP1. Kaplan-Meier survival curves were used for this analysis using XLSTAT, with the Greenwood IC and a significance level of 95%.

In order to assess the effect of GSTM3 and GSTP1 on CC cells, the expression of both proteins was inhibited by the use of antisense oligonucleotides (OAS). Three OAS were designed, two to specifically block proteins and one with a random sequence as a negative control (OAS-GSTM3, OAS-GSTP1 and OAS-Control). First, eight doses were evaluated for each OAS in culture with two cell lines, HeLa (cervical cancer) and HaCaT (negative cancer control). The doses used were from 10 to 1,280 ng/mL total incorporated into the culture medium. And subsequently, we evaluate cell proliferation at three different times at 24, 48 and 72 hours. In this experiment, it was observed that HaCaT cells were not affected by treatment with OAS-GSTM3 during the analysis period. Only a slight loss of survival was noted with the highest dose (1,280 ng/mL). In HeLa cells, viability losses were noted after 48 hours of treatment in all doses of OAS-GSTM3 (see FIG. 5A). After 72 hours, the highest treatment doses (640 and 1,280 ng/mL) showed a survival of less than 10% compared to the control cells. Similar results were obtained for treatment with OAS-GSTP1 in both cells.

Once the protein expression of the GSTM3 and/or GSTP1 in the tumor tissue is identified, the specific antisense oligonucleotides are administered (for example: 2′O-Me, 2′O-MOE, vivo-Morpholino, Morpholinos, LNA, PNA, among others) to perform the silencing of GSTs proteins, which will induce tumor cell death.

Although the present invention has been described with particularity in accordance with certain of the preferred embodiments, the examples should be interpreted only as illustrative of the invention and not for the purpose of limiting it. References cited herein are expressly incorporated for reference.

### BIBLIOGRAPHY

Adler, V., Yin, Z., Fuchs, S. Y., Benezra, M., Rosario, L., Tew, K. D., . . . Ronai, Z. (1999).
Regulation of JNK signaling by GSTp. The EMBO journal, 18(5), 1321-1334.
Akira, S., & Takeda, K. (2004). Toll-like receptor signalling. Nature reviews. Immunology, 4(7), 499-511.
Asea, A., Rehli, M., Kabingu, E., Boch, J. A., Baré, O., Auron, P. E., . . . Calderwood, S. K. (2002). Novel signal transduction pathway utilized by extracellular HSP70. Role of toll-like receptor (TLR) and TLR4. Journal of Biological Chemistry, 277(17), 15028-15034.
Atkinson, A. J., Colburn, W. A., DeGruttola, V. G., DeMets, D. L., Downing, G. J., Hoth, D. F., . . . Zeger, S. L. (2001). Biomarkers and surrogate endpoints: Preferred definitions and conceptual framework. Clinical Pharmacology and Therapeutics. https://doi.org/10.1067/mcp.2001.113989 Beavis, A. L., Gravitt, P. E., & Rositch, A. F. (2017). Hysterectomy-corrected cervical cancer mortality rates reveal a larger racial disparity in the United States. Cancer, 123(6), 1044-1050.
Bild, A. H., Potti, A., & Nevins, J. R. (2006). Linking oncogenic pathways with therapeutic opportunities. Nature Reviews Cancer.
Bosch, F. X., Lorincz, a, Munoz, N., Meijer, C. J. L. M., & Shah, K. V. (2002). The causal relation between human papillomavirus and cervical cancer. Journal of Clinical Pathology, 55(4), 244-265.
Brekke, O. H., & Sandlie, I. (2003). Therapeutic antibodies for human diseases at the dawn of the twenty-first century. Nature Reviews Drug Discovery.
Brewer, C. A., Setterdahl, J. J., Li, M. J., Johnston, J. M., Mann, J. L., & McAsey, M. E. (2000). Endoglin expression as a measure of microvessel density in cervical cancer. Obstetrics and Gynecology, 96(2), 224-228.
Burk, R. D., Chen, Z., Saller, C., Tarvin, K., Carvalho, A. L., Scapulatempo-Neto, C., . . . Mutch, D. (2017). Integrated genomic and molecular characterization of cervical cancer. Nature, 543(7645), 378-384.
Cabelguenne, A., Loriot, M. A., Stucker, I., Blons, H., Koum-Besson, E., Brasnu, D., . . . De Waziers, I. (2001). Glutathione-associated enzymes in head and neck squamous cell carcinoma and response to cisplatin-based neoadjuvant chemotherapy. International Journal of Cancer, 93(5), 725-730.
Cekanova, M., & Rathore, K. (2014). Animal models and therapeutic molecular targets of cancer: Utility and limitations. Drug Design, Development and Therapy. https://doi.org/10.2147/DDDT.S49584
Chang, J., Jung, H. H., Yang, J. Y., Lee, S., Choi, J., Im, G. J., & Chae, S. W. (2014). Protective effect of metformin against cisplatin-induced ototoxicity in an auditory cell line. JARO—Journal of the Association for Research in Otolaryngology, 15(2), 149-158.
Cho, W. C. S. (2007). Contribution of oncoproteomics to cancer biomarker discovery. Molecular Cancer.
Dasari, S., Wudayagiri, R., & Valluru, L. (2015). Cervical cancer: Biomarkers for diagnosis and treatment. Clinica Chimica Acta.
Denny, L. E. (2008). Prevention of cervical cancer. Reproductive health matters, 16(32), 18-31.
Dueñas-Gonzalez, A., Lizano, M., Candelaria, M., Cetina, L., Arce, C., & Cervera, E. (2005). Epigenetics of cervical cancer. An overview and therapeutic perspectives. Mol Cancer, 4, 38. https://doi.org/1476-4598-4-38[pii]\n10.1186/1476-4598-4-38
Encarnación, S., Guzmán, Y., Dunn, M. F., Hernández, M., Vargas, M. del C., & Mora, J. (2003).
Proteome analysis of aerobic and fermentative metabolism in Rhizobium etli CE3. In Proteomics (Vol 3, bll 1077-1085).
Gillet, E., Meys, J. F. A., Verstraelen, H., Verhelst, R., De Sutter, P., Temmerman, M., & Broeck, D. Vanden. (2012). Association between Bacterial Vaginosis and Cervical Intraepithelial Neoplasia: Systematic Review and Meta-Analysis. PLoS ONE, 7(10), e45201.
Green, J. A., Kirwan, J. M., Tierney, J. F., Symonds, P., Fresco, L., Collingwood, M., & Williams, C. J. (2001). Survival and recurrence after concomitant chemotherapy and radiotherapy for cancer of the uterine cervix: a systematic review and meta-analysis. Lancet, 358(9284), 781-786.
Hamburg, M. A., & Collins, F. S. (2010). The Path to Personalized Medicine—Perspective. The New England Journal of Medicine, 363(4), 301-304. https://doi.org/10.1056/NEJMp1006304 Hanahan, D., & Weinberg, R. A. (2000). The hallmarks of cancer. Cell, 100(1), 57-70.
Hanash, S. M., Pitteri, S. J., & Faca, V. M. (2008). Mining the plasma proteome for cancer biomarkers. Nature.
Hanash, S., & Taguchi, A. (2010). The grand challenge to decipher the cancer proteome. Nat Rev Cancer, 10(9), 652-660.
Hayden, M. S., & Ghosh, S. (2012). NF-kB, the first quarter-century: Remarkable progress and outstanding questions. Genes and Development, 26(3), 203-234.
Hayes, J. D., Flanagan, J. U., & Jowsey, I. R. (2005). Glutathione transferases. Annu Rev Pharmacol Toxicol, 45, 51-88.
Holohan, C., Van Schaeybroeck, S., Longley, D. B., & Johnston, P. G. (2013). Cancer drug resistance: an evolving paradigm. Nature Reviews Cancer, 13(10), 714-726.
Hu, Z., Zhu, D., Wang, W., Li, W., Jia, W., Zeng, X., . . . Ma, D. (2015). Genome-wide profiling of HPV integration in cervical cancer identifies clustered genomic hot spots and a potential microhomology-mediated integration mechanism. Nature Genetics, 47(2), 158-163.
Huang, J., Tan, P.-H., Thiyagarajan, J., & Bay, B.-H. (2003). Prognostic significance of glutathione Stransferase-pi in invasive breast cancer. Modern pathology: an official journal of the United States and Canadian Academy of Pathology, Inc, 16(6), 558-565.
Hurkman, W. J., & Tanaka, C. K. (1986). Solubilization of plant membrane proteins for analysis by two-dimensional gel electrophoresis. Plant physiology, 81(3), 802-806.
IARC. (2016). Globocan 2012. Http://Www-Dep.Iarc.Fr/, GLOBOCAN, 2012-2013.
Jankova, L., Robertson, G., Chan, C., Tan, K. L., Kohonen-Corish, M., Fung, C. L.-S., . . . Clarke, S.
J. (2012). Glutathione S-transferase Pi expression predicts response to adjuvant chemotherapy for stage C colon cancer: a matched historical control study. BMC cancer, 12(1), 196.
Josic, D., Clifton, J. G., Kovac, S., & Hixson, D. C. (2008). Membrane proteins as diagnostic biomarkers and targets for new therapies. Current Opinion In Molecular Therapeutics, 10(2), 116-123. Opgehaal van.
Junod, S. W. (2013). FDA and Clinical Drug assays: A Short History. Opgehaal van.
Kawano, M., Mabuchi, S., Matsumoto, Y., Sasano, T., Takahashi, R., Kuroda, H., . . . Kimura, T. (2015). The significance of G-CSF expression and myeloid-derived suppressor cells in the chemoresistance of cervical cancer. Scientific Reports, 5(April), 1-13.
Klose, J., & Kobalz, U. (1995). Two-dimensional electrophoresis of proteins: an updated protocol and implications for a functional analysis of the genome. Electrophoresis, 16(6), 1034-1059.
Kogo, R., How, C., Chaudary, N., Bruce, J., Shi, W., Hill, R. P., . . . Liu, F.-F. (2014). The microRNA-218-Survivin axis regulates migration, invasion, and lymph node metastasis in cervical cancer. Oncotarget, 6(2), 1090-1100.
Kueng, W., Silber, E., & Eppenberger, U. (1989). Quantification of cells cultured on 96-well plates.
Analytical Biochemistry, 182(1), 16-19.
Laborde, E. (2010). Glutathione transferases as mediators of signaling pathways involved in cell proliferation and cell death. Cell Death and Differentiation.
Loktionov, a, Watson, M. a, Gunter, M., Stebbings, W. S., Speakman, C. T., & Bingham, S. a. (2001). Glutathione-S-transferase gene polymorphisms in colorectal cancer patients: interaction between GSTM1 and GSTM3 allele variants as a risk-modulating factor. Carcinogenesis, 22(7), 1053-1060.
Louie, S. M., Grossman, E. A., Crawford, L. A., Ding, L., Camarda, R., Huffman, T. R., . . . Nomura, D. K. (2016). GSTP1 Is a Driver of Triple-Negative Breast Cancer Cell Metabolism and Pathogenicity. Cell Chemical Biology, 23(5), 567-578.
Makridakis, M., & Vlahou, A. (2010). Secretome proteomics for discovery of cancer biomarkers. Journal of Proteomics.
Mao, C., Hughes, J. P., Kiviat, N., Kuypers, J., Lee, S. K., Adam, D. E., & Koutsky, L. A. (2003). Clinical findings among young women with genital human papillomavirus infection. American Journal of Obstetrics and Gynecology, 188(3), 677-684.
Matsuzawa, A., Saegusa, K., Noguchi, T., Sadamitsu, C., Nishitoh, H., Nagai, S., . . . Ichijo, H. (2005).
ROS-dependent activation of the TRAF6-ASK1-p38 pathway is selectively required for TLR4-mediated innate immunity. Nature immunology, 6(6), 587-592.
Medeiros, R., Vasconcelos, A., Costa, S. Lopes, C. (2004). Metabolic Susceptibility Genes and Prostate Cancer Risk in a Southern European Population: The Role of Glutathione S-Transferases GSTM1, GSTM3, and GSTT1 Genetic Polymorphisms. Prostate, 58(4), 414-420.
Meding, S., Balluff, B., Elsner, M., Schöne, C., Rauser, S., Nitsche, U., . . . Walch, A. (2012). Tissuebased proteomics reveals FXYD3, S100A11 and GSTM3 as novel markers for regional lymph node metastasis in colon cancer. Journal of Pathology, 228(4), 459-470.
Mileo, A. M., Abbruzzese, C., Mattarocci, S., Bellacchio, E., Pisano, P., Federico, A., . . . Paggi, M. G. (2009). Human Papillomavirus-16 E7 Interacts with Glutathione S-Transferase P1 and Enhances Its Role in Cell Survival. PLoS ONE, 4(10).
Mitra, A., MacIntyre, D. A., Marchesi, J. R., Lee, Y. S., Bennett, P. R., & Kyrgiou, M. (2016). The vaginal microbiota, human papillomavirus infection and cervical intraepithelial neoplasia: What do we know and where are we going next? Microbiome. https://doi.org/10.1186/s40168-016-0203-0
Mitra, A. P., Pagliarulo, V., Yang, D., Waldman, F. M., Datar, R. H., Skinner, D. G., . . . Cote, R. J. (2009). Generation of a concise gene panel for outcome prediction in urinary bladder cancer. Journal of Clinical Oncology, 27(24), 3929-3937.
Monaci, P., Luzzago, A., Santini, C., De Pra, A., Arcuri, M., Magistri, F., . . . Cortese, R. (2008).
Differential screening of phage-Ab libraries by oligonucleotide microarray technology. PLoS ONE, 3(1).
Moyer, V. A. (2012). Screening for Cervical Cancer: U.S. preventive services task force recommendation statement. Annals of Internal Medicine.
Mueller-Klieser, W. (1997). Three-dimensional cell cultures: from molecular mechanisms to clinical applications. Am J Physiol, 273(4 Pt 1), C1109-23.
Nishitoh, H., Saitoh, M., Mochida, Y., Takeda, K., Nakano, H., Rothe, M., . . . Ichijo, H. (1998). ASK1 is essential for JNK/SAPK activation by TRAF2. Molecular cell, 2(3), 389-395.
Offit, K. (2011). Personalized medicine: New genomics, old lessons. Human Genetics.
Ortiz, A. R., Strauss, C. E. M., & Olmea, O. (2002). MAMMOTH (matching molecular models obtained from theory): an automated method for model comparison. Protein science: a publication of the Protein Society, 11(11), 2606-2621.
Owen, L. A., Uehara, H., Cahoon, J., Huang, W., Simonis, J., & Ambati, B. K. (2012). Morpholino-mediated increase in soluble fit-1 expression results in decreased ocular and tumor neovascularization. PLoS ONE, 7(3).
Pavlou, M. P., & Diamandis, E. P. (2010). The cancer cell secretome: A good source for discovering biomarkers? Journal of Proteomics.
Pectasides, D., Kamposioras, K., Papaxoinis, G., & Pectasides, E. (2008). Chemotherapy for recurrent cervical cancer. Cancer Treatment Reviews.
Peralta-Zaragoza, O., Bermúdez-Morales, V. H., Pérez-Plasencia, C., Salazar-León, J., Gómez-Cerón, C., & Madrid-Marina, V. (2012). Targeted treatments for cervical cancer: A review. OncoTargets and Therapy.
Posadas, E. M., Simpkins, F., Liotta, L. a, MacDonald, C., & Kohn, E. C. (2005). Proteomic analysis for the early detection and rational treatment of cancer—realistic hope? Annals of oncology: official journal of the European Society for Medical Oncology/ESMO, 16(1), 16-22.
Price, J. C., Guan, S., Burlingame, A., Prusiner, S. B., & Ghaemmaghami, S. (2010). Analysis of proteome dynamics in the mouse brain. Proceedings of the National Academy of Sciences of the United States of America, 107(32), 14508-14513.
Quail, D. F., & Joyce, J. A. (2013). Microenvironmental regulation of tumor progression and metastasis. Nat Med, 19(11), 1423-1437.
Rose, P. G., Bundy, B. N., Watkins, E. B., Thigpen, J. T., Deppe, G., Maiman, M. A., . . . Insalaco, S. (1999). Concurrent cisplatin-based radiotherapy and chemotherapy for locally advanced cervical cancer. N Engl J Med, 340(15), 1144-1153.
Rouillard, A. D., Gundersen, G. W., Fernandez, N. F., Wang, Z., Monteiro, C. D., McDermott, M. G., & Ma'ayan, A. (2016). The harmonizome: a collection of processed datasets gathered to serve and mine knowledge about genes and proteins. Database, 2016, baw100.
Scarinci, I. C., Garcia, F. a R., Kobetz, E., Partridge, E. E., Brandt, H. M., Bell, M. C., . . . Castle, P. E. (2010). Cervical cancer prevention: New tools and old barriers. Cancer, 116(11), 2531-2542.
Scherf, U., Ross, D. T., Waltham, M., Smith, L. H., Lee, J. K., Tanabe, L., . . . Weinstein, J. N. (2000). A gene expression database for the molecular pharmacology of cancer. Nature genetics, 24(3), 236-244.
Scholle, F., Bendt, K. M., & Raab-Traub, N. (2000). Epstein-Barr Virus LMP2A Transforms Epithelial Cells, Inhibits Cell Differentiation, and Activates Akt. Journal of virology, 74 (22), 10681-10689.
Schwede, T., Kopp, J., Guex, N., & Peitsch, M. C. (2003). SWISS-MODEL: An automated protein homology-modeling server. Nucleic Acids Research, 31(13), 3381-3385.
Shannon, P., Markiel, A., Ozier, O., Baliga, N. S., Wang, J. T., Ramage, D., . . . Ideker, T. (2003). Cytoscape: A software Environment for integrated models of biomolecular interaction networks. Genome Research, 13(11), 2498-2504.
Siddik, Z. H. (2003). Cisplatin: mode of cytotoxic action and molecular basis of resistance.
Oncogene, 22(47), 7265-7279.
Sima, N., Wang, S., Wang, W., Kong, D., Xu, Q., Tian, X., . . . Ma, D. (2007). Antisense targeting human papillomavirus type 16 E6 and E7 genes contributes to apoptosis and senescence in SiHa cervical carcinoma cells. Gynecologic Oncology, 106(2), 299-304.
Simpson, R. J., Connolly, L. M., Eddes, J. S., Pereira, J. J., Moritz, R. L., & Reid, G. E. (2000). Proteomic analysis of the human colon carcinoma cell line (LIM 1215): development of a membrane protein database. Electrophoresis, 21, 1707-1732.
Tabas-Madrid, D., Nogales-Cadenas, R., & Pascual-Montano, A. (2012). GeneCodis3: A non-redundant and modular enrichment analysis tool for functional genomics. Nucleic Acids Research, 40(W1).
Tew, K. D., & Townsend, D. M. (2011). Regulatory functions of glutathione S-transferase P1-1 unrelated to detoxification. Drug metabolism reviews, 43(2), 179-193.
Tomayko, M. M., & Reynolds, C. P. (1989). Determination of subcutaneous tumor size in athymic (nude) mice. Cancer Chemotherapy and Pharmacology, 24(3), 148-154.
Townsend, D. M., & Tew, K. D. (2003). The role of glutathione-S-transferase in anti-cancer drug resistance. Oncogene, 22(47), 7369-7375.
Twombly, R. (2002). New Pap Test Terminology, Management Guidelines Published. Journal of the National Cancer Institute, 94(12), 878-880.
Vabulas, R. M., Ahmad-Nejad, P., da Costa, C., Miethke, T., Kirschning, C. J., Hacker, H., & Wagner, H. (2001). Endocytosed HSP60s use TLR2 and TLR4 to activate the TIR signaling pathway in innate immune cells. The Journal of biological chemistry, 276(33), 31332-31339.
Valdespino, V. M., & Valdespino, V. E. (2006). Cervical cancer screening: state of the art. Current opinion in obstetrics & gynecology, 18(1), 35-40.
Villa, L. L. (2008). Assessment of new technologies for cervical cancer screening. The Lancet Oncology.
Vizcaíno, J. A., Csordas, A., Del-Toro, N., Dianes, J. A., Griss, J., Lavidas, I., . . . Hermjakob, H. (2016). 2016 update of the PRIDE database and its related tools. Nucleic Acids Research, 44(D1), D447-D456.
Waggoner, S. E. (2003). Cervical cancer. In Lancet (Vol 361, bll 2217-2225).
Waldmann, T. A. (2003). Immunotherapy: Past, present and future. Nature Medicine.
Walsh, M. C., Kim, G. K., Maurizio, P. L., Molnar, E. E., & Choi, Y. (2008). TRAF6 autoubiquitination-independent activation of the NF-kB and MAPK pathways in response to IL-1 and RANKL. PLoS ONE, 3(12).
Waterston, R. H., Lindblad-Toh, K., Birney, E., Rogers, J., Abril, J. F., Agarwal, P., . . . Mouse Genome Sequencing, C. (2002). Initial sequencing and comparative analysis of the mouse genome. Nature, 420(6915), 520-562.
Wu, Y., Fan, Y., Xue, B., Luo, L., Shen, J., Zhang, S., . . . Yin, Z. (2006). Human glutathione S-transferase P1-1 interacts with TRAF2 and regulates TRAF2-ASK1 signals. Oncogene, 25, 5787-5800.
Wulfkuhle, J. D., Liotta, L. A., & Petricoin, E. F. (2003). Early detection: Proteomic applications for the early detection of cancer. Nat Rev. Cancer, 3(4), 267-275.
Ye, Z., Song, H., Higgins, J. P. T., Pharoah, P., & Danesh, J. (2006). Five glutathione S-transferase gene variants in 23,452 cases of lung cancer and 30,397 controls: Meta-analysis of 130 studies. PLoS Medicine, 3(4), 524-534.
Yokota, J. (2000). Tumor progression and metastasis. Carcinogenesis, 21(3), 497-503.
Zagouri, F., Sergentanis, T. N., Chrysikos, D., Filipits, M., & Bartsch, R. (2012). Molecularly targeted therapies in cervical cancer. a systematic review. Gynecologic Oncology.
Zuris, J. A., Thompson, D. B., Shu, Y., Guilinger, J. P., Bessen, J. L., Hu, J. H., Liu, D. R. (2014). Cationic lipid-mediated delivery of proteins enables efficient protein-based genome editing in vitro and in vivo. Nature biotechnology, 33(1), 73-80.

1. Antisense oligonucleotides to inhibit the expression of glutathione S transferase proteins, for the manufacture of a drug adapted for the treatment of cancer, to be administrable in mammals previously diagnosed with cancer.

2. The antisense oligonucleotides according to claim 1, wherein the glutathione S transferases are GSTM3 and GSTP1.

3. The antisense oligonucleotides according to claim 1, wherein GSTM3 and GSTP1 are used as therapeutic targets and/or prognostic factors.

4. The antisense oligonucleotides according to claim 1, wherein said oligonucleotides include 15-50 nucleotides in length and bases 1-773 of GSTP1 and bases 1-4144 of GSTM3, with a similarity of 100-50% of both sequences.

5. The antisense oligonucleotides according to claim 4, wherein said oligonucleotides are 18-30 nucleotides in length.

6. The antisense oligonucleotides according to claim 4, wherein said oligonucleotides are 20-25 nucleotides in length.

7. The antisense oligonucleotides according to claims 4, 5 and 6, wherein the bases for GSTP1 are close to the start codon and for GSTM3 close to the start codon.

8. The antisense oligonucleotides according to claims 4, 5 and 6, wherein the similarity of both sequences is 100-80%.

9. The antisense oligonucleotides according to claims 4, 5 and 6, wherein the similarity of both sequences is 100-90%.

10. The antisense oligonucleotides according to claim 1, wherein the oligonucleotide is one having sugar modified bases, column or skeleton modifications, nucleobase modifications and general modifications in natural oligonucleotides.

11. The antisense oligonucleotides according to claim 1, wherein the oligonucleotides are selected from the following: 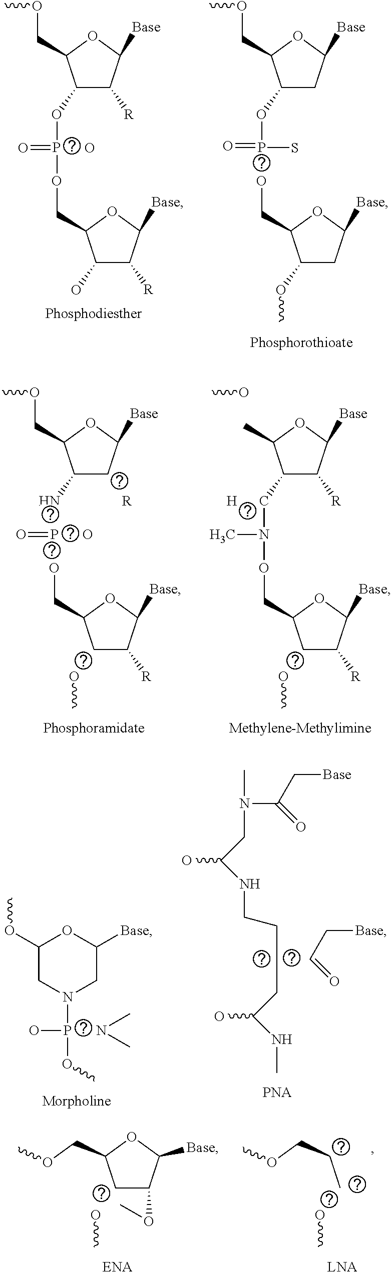 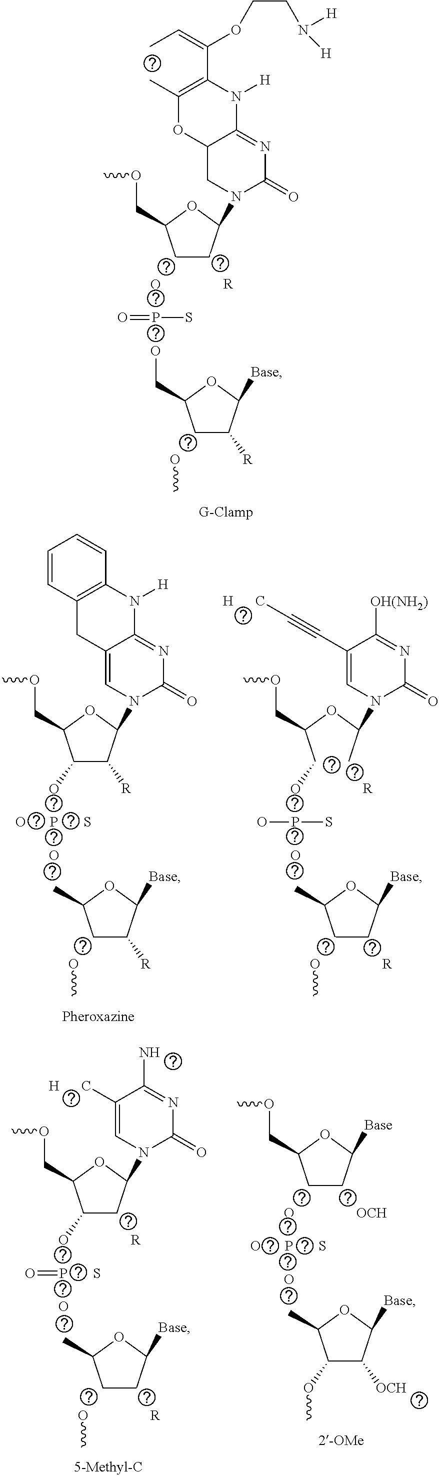 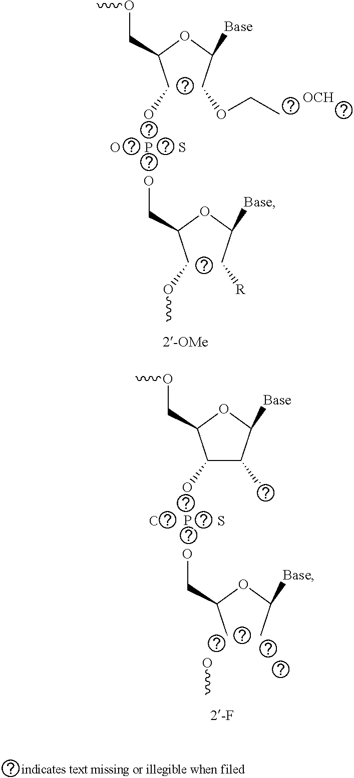
and/or have a chemical modification or a combination of the chemical modifications mentioned above.

12. The antisense oligonucleotides according to claim 11, wherein the preferred oligonucleotides have the following sequences:
anti-GSTM3
5′-TAG ACG ACT CGC ACG ACA TGG TGA C-3′;
anti-GSTP1
5′-AAT AGA CCA CGG TGT AGG GCG GCA T-3′;

13. The antisense oligonucleotides according to claim 1, wherein said oligonucleotides are directed to the messenger ribonucleic acids (mRNA) of the GSTM3 and GSTP1.

14. Antisense oligonucleotides according to any one of the preceding claims, wherein at least one or more oligonucleotides combined, are used to specifically block one protein or both proteins.

15. The antisense oligonucleotides according to claim 1, wherein the cancer is any wherein the tumor tissue contains one or both GSTM3 and GSTP1 proteins.

16. The antisense oligonucleotides according to claim 1, wherein the cancer is selected for lung cancer, breast cancer, colorectal cancer, prostate cancer, stomach cancer, liver cancer, esophageal cancer, cervical cancer, thyroid cancer, bladder cancer, non-Hodgkin lymphoma, pancreatic cancer, leukemia, kidney cancer, uterine body cancer, oropharyngeal cancer, brain and central nervous system cancer, ovarian cancer, melanoma cancer, gallbladder cancer, laryngeal cancer, multiple myeloma cancer, nasopharyngeal cancer, laryngopharyngeal cancer, Hodgkin lymphoma, testicular cancer, salivary gland cancer, vulvar cancer, Kaposi sarcoma cancer, penile cancer, mesothelioma, and vaginal cancer.

17. A kit for use in the identification of a subject to be treated with the oligonucleotides of claim 1, comprising at least one antisense oligonucleotide of glutathione S transferases, a protein extraction solution, at least two antibodies to the identification of proteins and optionally a secondary antibody, and a colorimetric developing solution for immunodetection assays such as: western staining, lateral flow membranes, ELISA or immunohistochemistry.

18. The kit according to claim 17, wherein the proteins to be identified are the GSTM3 and GSTP1 proteins.

19. A method for the identification of GSTs in vitro in samples of patients previously diagnosed with cancer comprising: a) extracting the protein from the tumor tissue, b) carrying out an analysis by immunodetection techniques and c) inhibiting the expression of the protein by use of the antisense oligonucleotide of claim 1.

20. A method for the treatment of cancer comprising a) the identification of GSTs in vitro in samples of patients previously diagnosed with cancer, b) the extraction of the protein from the tumor tissue, c) carrying out an analysis by immunodetection techniques and d) administering an antisense oligonucleotide directed to the messenger ribonucleic acids (mRNA) of the GSTM3 and GSTP1.


SEQUENCE LISTING (22 sequences)

SEQ ID NO: 1 - DNA, 76 bp, Artificial Sequence
  Description of Artificial Sequence Synthetic oligonucleotide
  M3-1
atgtcgtgcgagtcgtctatggttctcgggtactgggatattcgtgggctggcgcacgccatccgcctgctcctgg

SEQ ID NO: 2 - DNA, 78 bp, Artificial Sequence
  Description of Artificial Sequence Synthetic oligonucleotide
  M3-2
catagtcaggagcttccccgcacgtgtaccgtttctcctcataagaggtatccgtgaactccaggagcaggcggatgg

SEQ ID NO: 3 - DNA, 80 bp, Artificial Sequence
  Description of Artificial Sequence Synthetic oligonucleotide
  M3-3
ggaagctcctgactatgatcgaagccaatggctggatgtgaaattcaagctagacctggactttcctaatctgccctacc

SEQ ID NO: 4 - DNA, 79 bp, Artificial Sequence
  Description of Artificial Sequence Synthetic oligonucleotide
  M3-4
gcttgcgagcgatgtagcgcaagatggcattgctctgggtgatcttgttcttcccatccaggaggtagggcagattagg

SEQ ID NO: 5 - DNA, 75 bp, Artificial Sequence
  Description of Artificial Sequence Synthetic oligonucleotide
  M3-5
gctacatcgctcgcaagcacaacatgtgtggtgagactgaagaagaaaagattcgagtggacatcatagagaacc

SEQ ID NO: 6 - DNA, 79 bp, Artificial Sequence
  Description of Artificial Sequence Synthetic oligonucleotide
  M3-6
cagtttttcgtggtcagagctgtaacagagccttatcagttgtgtgcggaaatccattacttggttctctatgatgtcc

SEQ ID NO: 7 - DNA, 78 bp, Artificial Sequence
  Description of Artificial Sequence Synthetic oligonucleotide
  M3-7
gctctgaccacgaaaaactgaagcctcagtacttggaagagctacctggacaactgaaacaattctccatgtttctgg

SEQ ID NO: 8 - DNA, 72 bp, Artificial Sequence
  Description of Artificial Sequence Synthetic oligonucleotide
  M3-8
ggtgagaaaatccacaaaggtgagcttttccccggcaaaccatgagaatttccccagaaacatggagaattg

SEQ ID NO: 9 - DNA, 75 bp, Artificial Sequence
  Description of Artificial Sequence Synthetic oligonucleotide
  M3-9
cctttgtggattttctcacctatgatatcttggatcagaaccgtatatttgaccccaagtgcctggatgagttcc

SEQ ID NO: 10 - DNA, 74 bp, Artificial Sequence
  Description of Artificial Sequence Synthetic oligonucleotide
  M3-10
ccaagtgcctggatgagttcccaaacctgaaggctttcatgtgccgttttgaggctttggagaaaatcgctgcc

SEQ ID NO: 11 - DNA, 78 bp, Artificial Sequence
  Description of Artificial Sequence Synthetic oligonucleotide
  M3-11
ccactgggccatcttgttgttgatgggcatcttgcagaactgatcagactgtaagtaggcagcgattttctccaaagc

SEQ ID NO: 12 - DNA, 47 bp, Artificial Sequence
  Description of Artificial Sequence Synthetic oligonucleotide
  M3-12
ccatcaacaacaagatggcccagtggggcaacaagcctatatgctga

SEQ ID NO: 13 - DNA, 70 bp, Artificial Sequence
  Description of Artificial Sequence Synthetic oligonucleotide
  GSTM3-HindIII
atagacaagcttaacaaaatgtctgggtcgtcgcaccatcaccaccatcattcgtgcgagtcgtctatgg

SEQ ID NO: 14 - DNA, 32 bp, Artificial Sequence
  Description of Artificial Sequence Synthetic oligonucleotide
  GSTM3-BamHI
atacaaggatcctcagcatataggcttgttgc

SEQ ID NO: 15 - DNA, 24 bp, Artificial Sequence
  Description of Artificial Sequence Synthetic oligonucleotide
  E718-1
atgcatggacctaaggcaaccatt

SEQ ID NO: 16 - DNA, 18 bp, Artificial Sequence
  Description of Artificial Sequence Synthetic oligonucleotide
  E718-2*
ctgctgggatgcacacca

SEQ ID NO: 17 - DNA, 26 bp, Artificial Sequence
  Description of Artificial Sequence Synthetic oligonucleotide
  E7-18-Hind III
atacaaaagcttatgcatggacctaa

SEQ ID NO: 18 - DNA, 20 bp, Artificial Sequence
  Description of Artificial Sequence Synthetic oligonucleotide
  E7HPV18-his*
gatggtgatgatgctgctgg

SEQ ID NO: 19 - DNA, 25 bp, Artificial Sequence
  Description of Artificial Sequence Synthetic oligonucleotide
  Univ His-Tag BamH I*
tacgtggatcctagtggtgatggtg

SEQ ID NO: 20 - DNA, 25 bp, Artificial Sequence
  Description of Artificial Sequence Synthetic oligonucleotide
  anti-GSTM3
tagacgactcgcacgacatggtgac

SEQ ID NO: 21 - DNA, 25 bp, Artificial Sequence
  Description of Artificial Sequence Synthetic oligonucleotide
  anti-GSTP1
aatagaccacggtgtagggcggcat

SEQ ID NO: 22 - protein, 6 aa, Artificial Sequence
  Description of Artificial Sequence Synthetic 6xHis tag
HHHHHH